(19) United States
(12) Patent Application Publication
(10) Pub. No.: US 2022/0145295 A1
(43) Pub. Date: May 12, 2022

(54) ENZYMATIC RNA SYNTHESIS

(71) Applicant: President and Fellows of Harvard College, Cambridge, MA (US)

(72) Inventors: George M. Church, Cambridge, MA (US); Daniel J. Wiegand, Cambridge, MA (US); Richard E. Kohman, Cambridge, MA (US); Erkin Kuru, Cambridge, MA (US); Jonathan Rittichier, Cambridge, MA (US); Nicholas Conway, Cambridge, MA (US)

(73) Assignee: President and Fellows of Harvard College, Cambridge, MA (US)

(21) Appl. No.: 17/283,728

(22) PCT Filed: Oct. 11, 2019
(86) PCT No.: PCT/US2019/055870

§ 371 Date: Apr. 8, 2021

Related U.S. Application Data
(60) Provisional application No. 62/745,136, filed on Oct. 12, 2018.

(51) Int. Cl. C12N 15/113; C12P 19/34; C12N 9/12

(52) U.S. Cl. CPC C12N 15/113; C12P 19/34; C12N 9/1282; C12Y 207/07052; C12N 2310/322; C12N 2310/3231; C12N 2310/321

(57) ABSTRACT

Described herein are methods for the controlled de novo synthesis of RNA oligonucleotides using enzymatic catalysis. For example, provided herein are methods for preparing RNA oligonucleotides via controlled, template-independent addition of nucleotides to an initiator oligonucleotide 3′-terminus via enzymatic catalysis (also known as terminal transferase activity). Single nucleotides can be iteratively added by a compatible polymerase (e.g., a poly(N) polymerase such as a poly(U) polymerase) until a desired RNA oligonucleotide sequence is synthesized. Also provided are nucleotides and polymerases useful in the methods described herein.

### RELATED APPLICATIONS

This application claims priority under 35 U.S.C. § 119(e) to U.S. Provisional Application, U.S. Ser. No. 62/745,136, filed Oct. 12, 2018, the entire contents of which is incorporated herein by reference.

### GOVERNMENT SUPPORT

This invention was made with government support under grant number DE-FG02-02ER63445, awarded by the U.S. Department of Energy. The government has certain rights in the invention

### BACKGROUND OF THE INVENTION

Synthetic oligonucleotides are crucial to many aspects of biotechnological research in both the academic and industrial settings. Despite the high demand for longer, cheaper, and error-free oligonucleotides, current industrial leaders have not addressed the many limitations of traditional chemical synthesis methods developed decades ago. This is especially true for de novo RNA oligonucleotide synthesis, which remains largely inaccessible to those heavily invested in furthering genome engineering technologies, RNA-based diagnostics and therapeutics, RNA-based sequencing technologies, high-density nucleic acid-based information storage, and biological computing (Kaczmarek, Kowalski, and Anderson 2017). While there have been some improvements to the methods employed for the chemical synthesis of RNA oligonucleotides, the overall chemistry has changed very little since the late 1970s (Hughes and Ellington 2017). Chemical synthesis is plagued by many complicated reaction steps that require both harsh chemical reagents and biologically incompatible organic solvents. These reaction conditions lead to the depurination of the nucleotide bases, unexpected insertions or deletions from the overall sequence, and the preemptive irreversible capping of the oligonucleotide resulting in unwanted truncated products. This greatly increases the overall error-rate of synthesis, limits the maximum length of RNA oligonucleotides to less than 120 nucleotides, and requires longer lead-times to obtain appreciable yields of a desired product. Furthermore, the chemical synthesis of RNA oligonucleotide is extremely costly; compared to the current cost of DNA oligonucleotide synthesis at $0.1 per base (Carlson 2018), RNA synthesis is nearly 100-fold greater, not accounting for long or complex RNA oligonucleotides. Addressing the current limitations of RNA oligonucleotide synthesis is therefore important.

### SUMMARY OF THE INVENTION

Described herein are compounds, enzymes, compositions, systems, kits, and methods for the controlled de novo synthesis of RNA oligonucleotides using enzymatic catalysis. For example, provided herein are methods for preparing RNA oligonucleotides via controlled, template-independent addition of nucleotides to an initiator oligonucleotide's 3′-terminus via enzymatic catalysis (also known as terminal transferase activity). Single nucleotides can be iteratively added by a compatible polymerase (e.g., a poly(N) polymerase such as a poly(U) polymerase) until a desired RNA oligonucleotide sequence is synthesized.

The present disclosure is based on the discovery that certain polymerase enzymes can effectively catalyze template-independent terminal transferase reactions with a variety of modified and unmodified nucleotides. In one aspect, provided herein are methods for the synthesis of RNA oligonucleotides, wherein a poly(N) polymerase incorporates one or more nucleotides onto the 3′-terminus of an initiator oligonucleotide. It has been found that certain polymerases, such as poly(U) and poly(A) polymerases, among others, can catalyze terminal transferase reactions with a diversity of nucleotides, including modified nucleotides. After a poly(N) polymerase incorporates one or more nucleotides via terminal transferase, the process can be repeated in one or more in iterative steps, optionally with different nucleotides, until a desires RNA oligonucleotide sequence is obtained. Also provided herein are new poly(N) polymerase enzymes (e.g., mutant poly(U) polymerases) that are useful in the methods described herein.

In one aspect, proved herein are methods for preparing an RNA oligonucleotide comprising combining an initiator oligonucleotide, a poly(N) polymerase (e.g., poly(U) polymerase), and one or more modified nucleotides under conditions sufficient for the addition of at least one modified nucleotide to the 3′ end of the initiator oligonucleotide, thereby synthesizing an RNA oligonucleotide. The method may further comprise adding one or more additional nucleotides (modified or unmodified) to the resulting RNA oligonucleotide in iterative steps until a desired RNA oligonucleotide sequence is obtained. Also provided herein are compounds (e.g., modified nucleotides) that are useful in the methods described herein.

Other methods for controlled de novo RNA oligonucleotide synthesis are provided herein. For example, in another aspect, provided herein are methods wherein modified nucleotides (i.e., “reversible terminator oligonucleotides”, e.g., 2′- or 3′-O-protected nucleotides) are incorporated that reversibly alter the binding affinity of the polymerase (e.g., a poly(N) polymerase, such as poly(U)) to the extended initiator oligonucleotide. This change in binding affinity results in the termination of further nucleotide addition, producing a (n+1) oligonucleotide product that can be further extended after the modified group is restored to its natural state (e.g., a 2′- or 3′-OH group) via a mild deprotection chemistry. An “(n+1) oligonucleotide” is a product wherein a single nucleotide has been added to the initiator sequence. These methods are exemplified in the generic scheme shown in FIG. 2. In certain embodiments, the modified oligonucleotide is a 2′- or 3′-modified reversible terminator oligonucleotide. In certain embodiments, provided herein are methods of synthesizing an RNA oligonucleotide comprising combining an initiator oligonucleotide, a poly(N) polymerase (e.g., poly(U) polymerase), and a reversible terminator nucleotide (e.g., a 2′- or 3′-modified reversible terminator oligonucleotide) under conditions sufficient for the addition of the reversible terminator nucleotide to the 3′ end of the initiator oligonucleotide; followed by a step of deprotecting the resulting RNA oligonucleotide at the protected position of the reversible terminator nucleotide. Once deprotected, the resulting (n+1) extended RNA oligonucleotide can undergo subsequent terminal transferase reactions involving one or more modified or unmodified nucleotides until a desired RNA oligonucleotide sequence is obtained. Also provided herein are 2′- or 3′-modified reversible terminator oligonucleotides (e.g., 2′- or 3′-O-protected nucleotides) that are useful in the methods described herein.

In another aspect, provided herein are methods for RNA oligonucleotide synthesis employing non-hydrolyzable nucleotides. In these methods, the rate at which a polymerase incorporates nucleotides at the 3′-terminus of an initiator oligonucleotide is controlled by introducing a non-hydrolyzable nucleotide that competes for the enzyme's active site. These methods are exemplified in the generic scheme shown in FIG. 1. The rate of oligonucleotide synthesis is directly impacted by the ratio of the hydrolyzable nucleotides and the non-hydrolyzable nucleotides through competitive inhibition. In addition to the ratio of these nucleotides, other reaction parameters can be finely tuned to modulate the reaction velocity for a controlled synthesis. In certain embodiments, provided herein are methods of synthesizing an RNA oligonucleotide, the method comprising combining an initiator oligonucleotide, a poly(N) polymerase (e.g., a poly(U) polymerase), one or more nucleotides, and one or more non-hydrolyzable nucleotides under conditions sufficient for the addition of at least one hydrolyzable nucleotide to the 3′ end of the initiator oligonucleotide, wherein the concentration of the non-hydrolyzable nucleotides is sufficient to inhibit the rate of addition of the one or more nucleotides by the poly(N) polymerase; thereby synthesizing an RNA oligonucleotide. The method may further comprise adding one or more additional nucleotides (modified or unmodified) to the resulting RNA oligonucleotide until a desired RNA oligonucleotide sequence is obtained. Also provided herein are non-hydrolyzable nucleotides that are useful in the methods described herein.

In addition, provided herein are methods of ligating two oligonucleotides to yield an RNA oligonucleotides. In certain embodiments, the methods comprise providing a first oligonucleotide, wherein the first oligonucleotide comprises a 5′-triphosphate group; providing a second oligonucleotide; providing a poly(U) polymerase; combining the first and second oligonucleotides and the poly(U) polymerase under conditions sufficient for the ligation of the first oligonucleotide to the 3′ end of the second oligonucleotide. This embodiment is possible due to the discovery that 5′-triphosphate nucleotides with oligonucleotides at the 3′-positon are viable substrates for poly(N) polymerases (e.g., wild-type and mutated poly(U) polymerases) described herein.

In addition, RNA oligonucleotides produced by these methods can undergo reverse transcription (RT) to yield complementary DNA (e.g., cDNA) that is amplifiable by a DNA polymerase via the polymerase chain reaction (PCR).

Also provided herein are RNA oligonucleotides and DNA oligonucleotides produced by any method described herein.

The methods described herein involve enzymatic catalysis. Because enzymatic catalysis occurs under biologically compatible reaction conditions, unwanted degradation of the RNA molecule currently experienced by chemical synthesis can be eliminated. The present methods improve upon the current state of de novo RNA oligonucleotide synthesis, which is performed with phosphoramidite chemistry, often under harsh reaction conditions. The harsh reaction conditions of chemical RNA oligonucleotide synthesis makes it difficult and expensive to produce long RNA oligonucleotides, for example, >100 nucleotides in length If appreciable yields of a long RNA oligonucleotide are produced via chemical synthesis, it is possible that the error-rate of the oligonucleotide is very high. By using an enzymatic approach described herein, many of the current issues associated with long RNA oligonucleotide synthesis are solved. Applications of the methods described herein include the direct synthesis of RNA as well as material generation for nucleic acid nanotechnology, genome engineering techniques, and novel RNA and DNA therapeutics. In certain embodiments, methods described herein have the capacity to be miniaturized in a microfluidic format or performed in a highly parallelized manner such as micro-droplet printing. The methods provided herein can also be carried out in solid phase.

Also provided herein are compositions and kits comprising one or more of the poly(N) polymerases and/or nucleotides described herein.

The details of certain embodiments of the invention are set forth in the Detailed Description of Certain Embodiments, as described below. Other features, objects, and advantages of the invention will be apparent from the Definitions, Examples, Figures, and Claims.

### Definitions

### General Definitions

As used herein, the term “polymerase” generally refers to an enzyme that is capable of synthesizing RNA or DNA oligonucleotides. In some embodiments, a polymerase is capable of synthesizing an oligonucleotide in a template-dependent manner. In other embodiments, a polymerase is capable of synthesizing an oligonucleotide in a template-independent manner. In some embodiments, a polymerase is an RNA polymerase. In some embodiments, a polymerase is a DNA polymerase. In some embodiments, a polymerase is a reverse transcriptase. A polymerase may be derived from any source, e.g., recombinant polymerase, bacterial polymerase. In some embodiments, a polymerase is a poly(N) polymerases. In some embodiments, a polymerase is a poly(U), poly(A), poly(C), or poly(G) polymerase. In some embodiments, a polymerase is capable of adding a nucleotide, e.g., a nucleotide, to the 3′ end of an oligonucleotide, e.g., an initiator oligonucleotide. In some embodiments, a polymerase selectively adds a single nucleotide species, e.g., nucleotide comprising an uracil base in the case of poly(U) polymerases, to the 3′ end of an oligonucleotide, e.g., an initiator oligonucleotide.

As used herein, the term “RNA oligonucleotide” generally refers to a polymer of nucleotides, ribonucleotides, or analogs thereof. An RNA oligonucleotide can have any sequence. As used herein, an RNA oligonucleotide may have any three-dimensional structure, and may perform any function, known or unknown to one of skill in the art. An RNA oligonucleotide may be naturally occurring or synthetic. In some embodiments, a RNA oligonucleotide may be a messenger RNA (mRNA), a transfer RNA, ribosomal RNA, a short interfering RNA (siRNA), a short-hairpin RNA (shRNA), a micro-RNA (miRNA), a ribozyme, a recombinant oligonucleotide, a branched oligonucleotide, an isolated or synthetic RNA oligonucleotide of any sequence, a probe, and/or a primer. In some embodiments, an RNA oligonucleotide comprises nucleotides comprising naturally occurring bases, e.g., adenine or uracil. In some embodiments, an RNA oligonucleotide comprises non-naturally occurring or modified nucleotides, e.g., nucleotides comprising sugar modifications, base modifications, e.g., purine or pyrimidine modifications. In some embodiments, a RNA oligonucleotide comprises a combination of naturally, non-naturally occurring, and modified nucleotides. In some embodiments, a nucleotide may comprise at least one modified backbone or linkage, e.g., a phosphorothioates backbone or linkage. In some embodiments, a RNA oligonucleotide is single-stranded. In other embodiments, a RNA oligonucleotide is double-stranded. In some embodiments, a RNA oligonucleotide is synthesized via template-independent synthesis. In some embodiments, a RNA oligonucleotide is at least 5, at least 10, at least 20, at least 50, at least 100, at least 200, at least 300, at least 400, or at least 500 nucleotides in length.

As used herein, the term “DNA oligonucleotide” generally refers to a polymer of DNA nucleotides, deoxyribonucleotides, or analogs thereof. As used herein, a DNA oligonucleotide may have any three-dimensional structure, and may perform any function, known or unknown to one of skill in the art. A DNA oligonucleotide may be naturally occurring or synthetic. In some embodiments, a DNA oligonucleotide may be an exon, an intron, a cDNA sequence, a recombinant oligonucleotide, a branched oligonucleotide, a plasmid, a vectors, and/or an isolated DNA of any sequence. In some embodiments, a DNA oligonucleotide comprise DNA nucleotides comprising naturally occurring bases, e.g., adenine, cytosine, guanine, or thymine. In some embodiments, a DNA oligonucleotide comprise non-naturally occurring or modified DNA nucleotides, e.g., DNA nucleotides comprising sugar modifications, purine or pyrimidine modifications. In some embodiments, a DNA oligonucleotide comprises a combination of naturally, non-naturally occurring, and modified DNA nucleotides. In some embodiments, a DNA nucleotide may comprise at least one modified backbone or linkage, e.g., a phosphorothioates backbone or linkage. In some embodiments, a DNA oligonucleotide is single-stranded. In other embodiments, a DNA oligonucleotide is double-stranded. In some embodiments, a DNA oligonucleotide is synthesized via reverse transcription. In some embodiments, a DNA oligonucleotide is at least 5, at least 10, at least 20, at least 50, at least 100, at least 200 DNA, at least 300, at least 400, or at least 500 DNA nucleotides in length.

As used herein, the term “nucleotide” or “ribonucleotide” generally refers to a nucleotide monomer that comprises a ribose sugar, a phosphate group, and a nucleobase. An nucleotide may be naturally occurring, non-naturally occurring, or modified. In some embodiments, an nucleotide comprises a nucleobase or base, e.g., a purine or pyrimidine base. In some embodiments, a base is a naturally occurring base, e.g., adenine, cytosine, guanine, thymine, uracil, or inosine. In some embodiments, a nucleotide may comprise a non-naturally occurring nucleobase. In some embodiments, a nucleotide may comprise a modified nucleobase. In some embodiments, a nucleotide may comprise a modification of the ribose sugar, e.g., at the 2′ position, e.g., 2′-F, 2′-O-alkyl, 2′-amino, or 2′-azido. In some embodiments, a nucleotide is a non-hydrolyzable nucleotide, e.g., may comprise a modified triphosphate group. In certain embodiments, the modified nucleotide is a reversible terminator oligonucleotide, e.g., a 2′- or 3′-OH-protected nucleotide.

As used herein, the term “initiator oligonucleotide” generally refers to a short, single-stranded RNA oligonucleotide that is capable of initiating template-independent synthesis. An initiator oligonucleotide is, in certain embodiments, less than 20 nucleotides in length. In some embodiments, an initiator oligonucleotide is less than 20, less than 18, less than 15, less than 12, less than 10, less than 8, or less than 5 nucleotides in length. In some embodiments, an initiator oligonucleotide is 5, 6, 7, 8, 9, 10, 11, 12, 13, 14, 15, 16, 17, 18, 19, or 20 nucleotides in length. In some embodiments, an initiator oligonucleotide is labeled at its 5′ end, e.g., labeled with a fluorophore. In some embodiments, an initiator oligonucleotide is attached to a substrate at its 5′ end. In some embodiments, a substrate may be a glass surface, a bead, a biomolecule, or any conceivable substrate suitable for template-independent synthesis.

As used herein, the term “template-independent” generally refers to the synthesis of a RNA oligonucleotide that does not require a template DNA oligonucleotide. Template-independent synthesis will generally comprise the use of an initiator oligonucleotide and a polymerase, e.g., a poly(N) polymerase. Oligonucleotides, e.g., RNA oligonucleotides, synthesized using template-independent synthesis are generally synthesized by adding nucleotides, e.g., nucleotides, to the 3′ end of an existing oligonucleotide, e.g., an initiator oligonucleotide.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer, or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

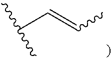

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C_{3_8} cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted. In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R—, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring;

each instance of R^{dd} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl;

or two geminal R^{dd} substituents can be joined to form ═O or ═S.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb})₂)₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group. The term “monosubstituted amino” refers to an amino group wherein the nitrogen atom directly attached to the parent molecule is substituted with one hydrogen and one group other than hydrogen, and includes groups selected from —NH(R^{bb}) —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb}, and R^{cc} are as defined herein, and wherein R^{bb} of the group —NH(R^{bb}) is not hydrogen. The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen. The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “thio” or “thiol” refers to the group —SH. The term “substituted thio” or “substituted thiol,” by extension, refers to a thiol group wherein the sulfur atom directly attached to the parent molecule is substituted with a group other than hydrogen. In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R—, —C(═O)N(R^{bb})₂, —C(═NR^{bb})R—, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR)₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “amino acid” refers to a molecule containing both an amino group and a carboxyl group. Amino acids include alpha-amino acids and beta-amino acids, the structures of which are depicted below. In certain embodiments, an amino acid is an alpha amino acid.

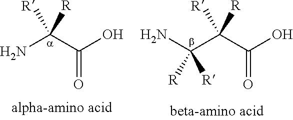

Suitable amino acids include, without limitation, natural alpha-amino acids such as D- and L-isomers of the 20 common naturally occurring alpha-amino acids found in peptides (e.g., A, R, N, C, D, Q, E, G, H, I, L, K, M, F, P, S, T, W, Y, V, as provided below), unnatural alpha-amino acids natural beta-amino acids (e.g., beta-alanine), and unnatural beta-amino acids. Exemplary natural alpha-amino acids include L-Alanine (A), L-Arginine (R), L-Asparagine (N), L-Aspartic acid (D), L-Cysteine (C), L-Glutamic acid (E), L-Glutamine (Q), Glycine (G), L-Histidine (H), L-Isoleucine (I), L-Leucine (L), L-Lysine (K), L-Methionine (M), L-Phenylalanine (F), L-Proline (P), L-Serine (S), L-Threonine (T), L-Tryptophan (W), L-Tyrosine (Y), and L-Valine (V). Exemplary unnatural alpha-amino acids include D-Arginine, D-Asparagine, D-Aspartic acid, D-Cysteine, D-Glutamic acid, D-Glutamine, D-Histidine, D-Isoleucine, D-Leucine, D-Lysine, D-Methionine, D-Phenylalanine, D-Proline, D-Serine, D-Threonine, D-Tryptophan, D-Tyrosine, D-Valine, Di-vinyl, α-methyl-Alanine (Aib), α-methyl-Arginine, α-methyl-Asparagine, α-methyl-Aspartic acid, α-methyl-Cysteine, α-methyl-Glutamic acid, α-methyl-Glutamine, α-methyl-Histidine, α-methyl-Isoleucine, α-methyl-Leucine, α-methyl-Lysine, α-methyl-Methionine, α-methyl-Phenylalanine, α-methyl-Proline, α-methyl-Serine, α-methyl-Threonine, α-methyl-Tryptophan, α-methyl-Tyrosine, α-methyl-Valine, Norleucine, terminally unsaturated alpha-amino acids and bis alpha-amino acids (e.g., modified cysteine, modified lysine, modified tryptophan, modified serine, modified threonine, modified proline, modified histidine, modified alanine, and the like). There are many known unnatural amino acids any of which may be included in the peptides of the present invention. See for example, S. Hunt, The Non-Protein Amino Acids: In Chemistry and Biochemistry of the Amino Acids, edited by G. C. Barrett, Chapman and Hall, 1985.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, protecting groups (e.g., nitrogen or oxygen protecting groups) such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Protecting groups (e.g., nitrogen or oxygen protecting groups) such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Protecting groups (e.g., nitrogen or oxygen protecting groups) such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other protecting groups (e.g., nitrogen or oxygen protecting groups) include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In certain embodiments, a protecting group (e.g., nitrogen or oxygen protecting group) is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds).

As used herein, the term “salt” refers to any and all salts, and encompasses pharmaceutically acceptable salts. The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

### BRIEF DESCRIPTION OF THE DRAWINGS

The accompanying drawings, which constitute a part of this specification, illustrate several embodiments of the invention and together with the description, serve to explain the principles of the invention.

FIG. 1. Reaction diagram for the controlled incorporation of natural ribonucleotide triphosphates (rNTPs) at the 3′-terminus of an initiator oligonucleotide under impeding reaction conditions. The rate of rNTP incorporation (Kₙ) is controlled through the addition of non-hydrolyzable or incompatible nucleotides that act as competitive inhibitors of the hydrolyzable nucleotide incorporation, such as those with modifications of the α-, β-, or γ-phosphate of the triphosphate. The number of incorporation events (where n=zero to hundreds) is determined by the percentage of non-hydrolyzable or incompatible nucleotides (0%-100%) present in the extension reaction, whereby higher percentages of competitive inhibitor nucleotides limits the length of the RNA oligonucleotide produced by the reaction. When the initiator oligonucleotide is attached to a surface through a cleavable covalent linkage (X), the RNA oligonucleotide base composition can be variable through rapid switching of the reaction conditions.

FIG. 2. Reaction diagram for the incorporation of modified rNTPs to the 3′-terminus of an initiator oligonucleotide that limits the extension reaction to the addition of just 1 nucleotide. The incorporation of the modified rNTP reversibly prevents further extension events until the extended oligonucleotide is treated with a gentle RNA tolerant deprotectant to yield the natural hydroxyl groups. When the initiator oligonucleotide is attached to a surface through a cleavable covalent linkage (X), the growing RNA oligonucleotide can be iteratively extended without the need of purification to remove nucleotide from the previous incorporation event. Reversible terminator rNTPs can have, for example, non-natural chemical domains at the 2′-, 3′-, or 2′- and 3′-positions (R and R′) of the nucleotide in addition to modifications to other sites on the nucleotide (e.g., the nucleotide base). Each modification can be further derivatized to include a linker and fluorophore in order to optically verify the (n+1) incorporation event after enzymatic catalysis before gentle deprotection treatment and subsequent extension.

FIGS. 3A to 3C. FIG. 3A shows a bar plot of initial activity screen of polymerase μ R387K with various divalent cations and combinations of divalent cations with decreasing concentrations with 200 μM dNTP. Denaturing gel electrophoresis analysis of dATP incorporation (FIG. 3B) and rATP incorporation (FIG. 3C) at nucleotide concentrations of 200-50 μM and 5.0-0.62 mM for dATP and rATP, respectively. These reactions were supplemented with 0.25 mM Mn²⁺ and Mg²⁺. The control reaction consisted of all reaction components except nucleotide. For all reactions, the initiator oligonucleotide was a HPLC-purified poly dT-15-mer.

FIGS. 4A and 4B. FIG. 4A shows a denaturing gel electrophoresis analysis of modified nucleotide (2′-amino-rATP, 2′-O-methyl-rATP 2′-F-rATP, & 2′-azido-rATP) incorporation by S. cerevisiae poly(A) polymerase. All 2′-modified ribonucleotides were at a final concentration of 2.5 mM and incubated at 37° C. for 60 minutes. The control reaction consisted of all reaction components except nucleotide. FIG. 4B shows a denaturing gel electrophoresis analysis of reversible terminator 2′-O-allyl-ATP incorporation by S. cerevisiae poly(A) polymerase over a range of nucleotide concentrations (250 μM to 4000 μM) incubated at 37° C. for 60 minutes. The negative reaction consisted of all reaction components except nucleotide.

FIGS. 5A and 5B. FIG. 5A shows a denaturing gel electrophoresis analysis of natural ribonucleotide incorporation by S. pombe poly(U) polymerase. All of the natural ribonucleotides were at a final concentration of 1.0 mM and incubated at 37° C. for 30 minutes. The control reaction consisted of all reaction components except nucleotide. FIG. 5B shows kinetic analysis of natural ribonucleotide incorporation by S. pombe poly(U) polymerase; measures the total concentration of single-stranded RNA as a function of time. Error bars indicate the standard deviation from the mean with N=3.

FIG. 6. Denaturing gel electrophoresis analysis of modified ribonucleotide (2′-O-methyl) incorporation by S. pombe poly(U) polymerase. All modified ribonucleotides were at a final concentration of 2.5 mM and incubated at 37° C. for 60 minutes. The control reactions consisted of all reaction components except nucleotide.

FIGS. 7A and 7B. FIG. 7A shows a denaturing gel electrophoresis analysis of natural ribonucleotide incorporation by S. pombe poly(U) polymerase in the presence of two different initiator oligonucleotides (5′-FAM-rA-15-mer and 5′-Cy5-rU-15-mer). All four natural ribonucleotides were at a final concentration of 1.0 mM and incubated at 37° C. for 30 minutes. The control reaction consisted of all reaction components except nucleotide for each initiator oligonucleotide. FIG. 7B shows a denaturing gel electrophoresis analysis of reversible terminator 2′-O-allyl-ATP or -UTP incorporation using initiator oligonucleotides with secondary structure via strong hairpin formation. Each oligonucleotide was similar in sequence except that the location of the hairpin in comparison to the 3′-terminus was varied to produce the following: 1 base from the 3′-terminus (H1), 5 bases from the 3′-terminus (H5), 10 bases from the 3′-terminus (H10), and 20 bases from the 3′-terminus. Sequence base composition is shown. In order to ensure that oligonucleotides' hairpins were correctly formed before enzymatic extension, the oligonucleotides were heated to 95° C. and then slowly cooled at a rate of 0.1° C./min to 15° C. in the appropriate enzymatic reaction buffer on a thermocycler. After cooling, the remaining reaction components were added to the hairpin initiator oligonucleotide and the extension reaction was carried out for 5 minutes at 37° C.

FIGS. 8A to 8C. FIG. 8A shows denaturing gel electrophoresis analysis of S. pombe poly(U) polymerase RNA synthesis reactions with (+) and without (−) inorganic pyrophosphatase (PPi-ase) for each of the natural ribonucleotides. The control reaction consisted of all reaction components except nucleotide. Kinetic analysis demonstrating the increased rate of ATP (FIG. 8B) and UTP (FIG. 8C) incorporation by S. pombe poly(U) polymerase in the presence of PPi-ase.

FIGS. 9A and 9B. FIG. 9A shows a denaturing gel electrophoresis analysis of S. pombe poly(U) polymerase RNA synthesis reactions with un-modified rUTP and based-modified Pseudouridine (PsUTP) at varying concentrations of nucleotide. Reactions were incubated at 37° C. for 30 minutes. FIG. 9B shows denaturing gel electrophoresis analysis of S. pombe poly(U) polymerase RNA synthesis reactions with an array of nucleoside triphosphates with a modified adenosine, uridine, cytidine, guanosine, or the universal base inosine. The indicated list number corresponds to the gel lane where activity of poly(U) polymerase was analyzed in the presence of 1 mM of each individual nucleotide. For comparison purposes, the natural nucleoside triphosphates were incubated and analyzed in parallel. Control reactions, denoted with a “C” on the gels, indicate those reactions with all RNA synthesis reaction components with the exception of nucleoside triphosphate. The initiator oligonucleotide was a poly-rU-15mer with a 5′-Cy5 fluorophore. All reactions were incubated at 37° C. for 30 minutes.

FIG. 10. Denaturing gel electrophoresis analysis of S. pombe poly(U) polymerase RNA synthesis reactions incubated with increasing concentrations of the non-hydrolyzable ribonucleotide Uridine-5′-[(α,β)-imido]triphosphate and UTP. Reactions were incubated for 30 minutes at 37° C. and the control reaction consisted all reaction components except the non-hydrolyzable ribonucleotide.

FIGS. 11A to 11E. FIG. 11A shows a denaturing gel electrophoresis analysis of S. pombe poly(U) polymerase RNA synthesis reactions incubated with 2′-O-allyl-ATP, a 2′-blocked reversible terminator, at a concentration 1 mM with either a poly-rU-15-mer or poly-rA-15-mer initiator oligonucleotide. To demonstrate enzymatic activity, a control reaction supplemented with 2′-O-methyl-ATP (previously confirmed to be incorporated by S. pombe poly(U) polymerase) is shown. Additionally, a negative control reaction, which was supplemented with all reaction components except nucleoside triphosphate, was included on the denaturing gel. For reversible terminator reactions, the second band indicates positive incorporation resulting in a (n+1) extension event as compared to the negative control reaction. All RNA synthesis reactions were incubated with 10 pmol of initiator oligonucleotide at 37° C. for 30 minutes. FIG. 11B shows a denaturing gel electrophoresis analysis of the kinetics of 2′-O-allyl-ATP reversible terminator incorporation by S. pombe poly(U) polymerase. Reactions were incubated for 0.5, 5, 10, 30, and 60 minutes at 37° C. For each timepoint, a negative control was included with all reaction components with the exception of the 2′-O-allyl-ATP reversible terminator and is indicated on the gel with a minus (−) sign. Reactions incubated with the 2′-O-allyl-ATP reversible terminator are indicated on the gel with a plus (+) sign. For all reactions, the initiator oligonucleotide was a poly-rU-15-mer labeled with a 5′-Cy5 fluorophore. FIG. 11C shows a denaturing gel electrophoresis analysis of the optimization of buffer composition and pH in the biocompatible deblocking of the incorporated the 2′-O-allyl-ATP reversible terminator. Deblocking reactions were incubated at 50° C. for 10 minutes, oligonucleotide material was then purified, concentrated, and then further extended using poly(U) polymerase best reaction conditions. For each buffer, a negative control was included with all reaction components with the exception of the 2′-O-allyl-ATP reversible terminator and is indicated on the gel with a minus (−) sign. Reactions incubated with the 2′-O-allyl-ATP reversible terminator are indicated on the gel with a plus (+) sign. The starting material, indicated by an (S), was included in order to visualize the full synthesis cycle. A high-resolution denaturing gel electrophoresis analysis of the synthesis cycle from starting material (S or n+0) to (n+2) is shown in FIG. 11D. Like before, a negative control was included with all reaction components with the exception of the 2′-O-allyl-ATP reversible terminator and is indicated on the gel with a minus (−) sign. FIG. 11E shows a denaturing gel electrophoresis analysis of an (n+5) oligonucleotide synthesis using S. pombe poly(U) polymerase and the 2′-O-allyl-ATP reversible terminator. Each cycle consisted of a bulk solution extension reaction for 1 minute at 37° C. and a bulk solution deblocking reaction using optimized conditions for 10 minutes at 50° C. After each cycle, a small aliquot of material was set aside for gel analysis. The (n+0) starting material was 20-nt in length and the (n+5) final product was 25-nt in length.

FIGS. 12A and 12B. FIG. 12A shows a denaturing gel electrophoresis analysis of the (n+1) incorporation of the 2′-O-allyl-ATP, -UTP, -CTP, and -GTP reversible terminator nucleoside triphosphates using S. pombe poly(U) polymerase. All extension reactions were treated similarly, being incubated with 1 mM nucleotide for 1 minute at 37° C. The control reaction contained all reaction components except nucleotide. FIG. 12B shows a denaturing gel electrophoresis analysis of binary (n+2) synthesis using combinations of 2′-O-allyl-ATP and -UTP reversible terminators. The following combinations were tested: (n+2) A-A, (n+2) A-U, (n+2) U-A, and (n+2) U-U using optimized enzymatic extension and deblocking reaction conditions in bulk solution. The (n+1) reactions for 2′-O-allyl-ATP and -UTP reversible terminators are shown for comparison.

FIGS. 13A and 13B. FIG. 13A shows a denaturing gel electrophoresis analysis of the expression and purification of a N-terminus His⁶-tagged S. pombe poly(U) polymerase as indicated by the bright band with square under the “purified” lane. The expected molecular weight of the expressed N-terminus His⁶-tagged S. pombe poly(U) polymerase is approximately 45 kDa. FIG. 13B shows a denaturing gel electrophoresis analysis of N-terminus His⁶-tagged S. pombe poly(U) polymerase activity through the incorporation of the 2′-O-allyl-ATP reversible terminator nucleoside triphosphate. Reactions were supplemented with increasing amounts of initiator oligonucleotide (from 20 pmol to 1000 pmol) to determine the relative conversion rates as a function of initiator oligonucleotide material as performed by concentrated N-terminus His⁶-tagged S. pombe poly(U) polymerase. All reaction volumes were 10 μL, the initiator oligonucleotide was a poly-rU-15-mer labeled with a 5′-Cy5 fluorophore. Reactions were incubated at 37° C. for 30 seconds. The control reaction contained all reaction components (initiator oligonucleotide was 20 pmol) with the exception of reversible terminator nucleotide.

FIGS. 14A and 14B. FIG. 14A shows the development of a solid-phase support system for enzymatic RNA oligonucleotide system. 5′-amine initiator oligonucleotide were labeled with a Biotin-PEG-NHS linker, which allows the initiator oligonucleotide to be anchored to a streptavidin surface in a vessel such as a microplate well, beads, glass slides, etc. A denaturing gel electrophoresis analysis of oligonucleotide labeling is shown and streptavidin functionalized beads were used to perform quality control. Bound oligonucleotide included an internal Cy5 dye, which can be visualized using fluorescence microscopy. FIG. 14B shows a denaturing gel electrophoresis analysis of solid-phase RNA oligonucleotide synthesis using S. pombe poly(U) polymerase. Synthesis was conducted on beads in separate reaction vessel, one for a (n+1), (n+2) and (n+3). The sequence synthesized was +ACU in the (n+3) example. Extension reactions were carried out at 37° C. for 1 minute and deblocking reactions at 50° C. for 10 minutes. Beads were washed with 10 mM Tris-HCl (pH 6.5) in between extensions and deblocking.

FIGS. 15A to 15D. FIG. 15A depicts an exemplary scheme for the generation and use of a reusable solid-phase support system for enzymatic RNA oligonucleotide synthesis. Briefly, a solid support such as beads, wells, or slides is covalently derivatized with an appropriate linker bound to an initiator oligonucleotide containing a riboinosine (rI) or deoxyinosine (dI), preferably at the 3′-terminus. Solid-phase enzymatic RNA oligonucleotide synthesis is conducted to produce the desired product and then Endonuclease V is allowed to incubate with the full oligonucleotide (initiator+product). This will cleave the oligonucleotide product from the solid-support, leaving the riboinosine (rI) or deoxyinosine (dI) intact on the solid-support to be reused for future synthesis reactions. If desired, S. pombe poly(U) polymerase can be used to introduce the riboinosine (rI) to the 3′-terminus of the anchored initiator oligonucleotide using 2′-O-allyl versions of this nucleobase. FIG. 15B shows a denaturing gel electrophoresis analysis of Endonuclease V cleavage of an initiator RNA oligonucleotide containing a deoxyinosine (dI) in bulk and from the surface of amine functionalized silica beads. An example oligonucleotide initiator sequence is shown as well as a dual NHS-PEG linker, which was used to covalently anchor the 5′-amine oligonucleotide to the surface of amine silica beads. Endonuclease V cleavage was performed using the appropriate buffer for 1 hour at 37° C. and immediately run on the denaturing gel. FIG. 15C shows a denaturing gel electrophoresis analysis of S. pombe poly(U) polymerase controlled and uncontrolled extension using the reversible terminator 2′-O-allyl-ATP and a mixture of rNTPs, respectively, on new, non-cleaved solid-phase support and Endonuclease V cleaved solid-phase support. Extension reactions were supplemented with 1 mM of nucleotide or nucleotide mixture and incubated for 15 minutes at 37° C. Beads were washed with 10 mM Tris-HCl (pH 6.5) before Endonuclease V cleavage. Endonuclease V cleavage was performed using the appropriate buffer for 1 hour at 37° C. and immediately run on the denaturing gel. FIG. 15D shows a denaturing gel electrophoresis analysis of S. pombe poly(U) polymerase controlled synthesis of an (n+2) product using the covalently bound, Endonuclease V cleavable initiator oligonucleotide with the 2′-O-allyl-ATP reversible terminator nucleoside triphosphate. Extension reactions were supplemented with 1 mM of nucleotide and incubated at 37° C. for 15 minutes. Deblocking reactions were performed at 50° C. for 10 minutes. Beads were washed with 10 mM Tris-HCl (pH 6.5). Endonuclease V cleavage was performed using the appropriate buffer for 1 hour at 37° C. and immediately run on the denaturing gel. The control reaction that was extended to an (n+2) and incubated in the presence of Endonuclease V, but contained an anchored Cy5 initiator oligonucleotide that did not bear a riboinosine (rI) or deoxyinosine (dI). This was used to demonstrate that the oligonucleotide did not leech during Endonuclease V cleavage. The beads with the Cy5 initiator oligonucleotide remaining visibly blue after each synthesis cycle.

FIGS. 16A and 16B. FIG. 16A depicts a prototype enzymatic RNA oligonucleotide synthesizer with the capability to synthesize 4× oligonucleotides simultaneously at large reaction scales using a solid-phase support such as initiator oligonucleotide derivatized silica or magnetic beads. Syringe barrels are filled with enough solid support to meet the desired scale requirements of the synthesis run. Before loading the solid-phase support, a filter is placed at the bottom of the syringe barrel and bonded in place. This filter keeps the solid-phase support in place while being able to remove liquid from the syringe barrel. A typical synthesis cycle consists of an extension reaction, a wash step, a deblocking reaction, and then a final wash step. This process is repeated until the desired oligonucleotide is completed. To cleave the final product from the solid support a chemical or biological cleavage reagent is added to each syringe barrel, incubated if needed for a pre-determined time, eluted, and collected via filtration. If the solid-phase support is to be reused, if for example Endonuclease V was used to cleave the final oligonucleotide product, it can be left in the syringe barrel and primed for the next synthesis run. To heat the reactors, syringe barrels may be removed, capped at both ends, and placed into an incubator for the desired amount of time. Alternatively, heated jackets may be placed around the syringe barrels. A major advantage to enzymatic based RNA oligonucleotide synthesis is the recycling of materials (enzyme, nucleotide, etc.). FIG. 16B depicts a dual valving system that one can control in order to direct the liquid in the syringe barrels to either waste collection or recycle collection. The recycled components may be directly applied to the next cycle of oligonucleotide synthesis or be purified and stored for future synthesis runs. A similar setup and synthesis reactor can be scaled up to accommodate industrial level oligonucleotide manufacturing.

FIG. 17. FIG. 17 depicts an exemplary scheme for the synthesis of the reversible terminator nucleoside triphosphate 2′-O-allyl-ATP. The starting material nucleoside may be swapped for any of the natural bases (U, T, G, C) and/or desired modified base. The triphosphate may also be swapped out for a phosphorothioate at the alpha phosphate.

FIGS. 18A to 18D show gel electrophoresis analysis of H336 mutants' capacity to incorporate the natural nucleotide GTP—“G” and CTP—“C”. Blank reactions were supplemented with all components except enzyme and nucleotide. All reactions were incubated with 1 mM nucleotide, 5 pmol initiator oligonucleotide, and 1 μg of enzyme for 30 minutes at 37° C. A 15% TBE-Urea denaturing gel was used for extension reaction analysis.

FIGS. 19A to 19F show gel electrophoresis analysis of poly(U) polymerase mutant H336R capacity to incorporate an array of natural and analogue nucleotides in comparison to the wild-type poly(U) polymerase. FIGS. 19A, 19D depict extension results for ATP based nucleotides for the wild-type and H336R mutant, respectively. FIGS. 19B, 19E depict extension results for UTP and ITP based nucleotides for the wild-type and H336R mutant, respectively. FIGS. 19C, 19F depict extension results for CTP and GTP based nucleotides for the wild-type and H336R mutant, respectively. All reactions were incubated with 1 mM nucleotide, 5 pmol initiator oligonucleotide, and 1 μg of enzyme for 30 minutes at 37° C. A 15% TBE-Urea denaturing gel was used for extension reaction analysis.

FIG. 20 shows uncontrolled incorporation of 2′-methoxy-adenosine triphosphate (2′-O-Me-ATP) by various S. pombe poly(U) polymerase mutants, specifically at position H336; single mutants are shown here in comparison to the wild-type (WT). Blank reactions contain all components except enzyme. Samples were analyzed with a 15% TBE-urea gel under denaturing conditions.

FIG. 21 shows uncontrolled incorporation of 2′-fluoro-adenosine triphosphate (2′-F-ATP) by various S. pombe poly(U) polymerase mutants, specifically at position N171; single mutants are shown here in comparison to mutant H336R. Blank reactions contained all reaction components except enzyme. Samples were analyzed with a 15% TBE-urea gel under denaturing conditions.

FIG. 22 shows controlled incorporation (capping) of 3′-methoxy-adenosine triphosphate (3′-O-Me-ATP) by various S. pombe poly(U) polymerase mutants, specifically at position N171; single mutants are shown here in comparison to mutant H336R and the wild-type. Top band indicates (n+1) product. Note: the wild-type sample indicates positive incorporation, however severe pyrophosphorolysis occurs. Negative reactions contained all reactions components except enzyme. Samples were analyzed using a 15% TBE-Urea gel under denaturing conditions.

FIG. 23 shows controlled incorporation of the reversible terminator 3′-O-allyl Adenosine Triphosphate (3′-(O-allyl)-ATP) by various S. pombe Mutants. Negative reactions contained all reactions components except enzyme. Samples were analyzed using a 15% TBE-Urea gel under denaturing conditions.

FIG. 24 shows controlled incorporation of the reversible terminator 3′-O-allyl carbonate deoxyadenosine triphosphate (3′-(0-allyl carbonate)-dATP) by the poly(U) polymerase double mutant H336R-N171A. Gel image indicates varying input amounts of initiator oligonucleotide (2 pmol/rxn, 5 pmol/rxn, and 10 pmol/rxn) with increasing amounts of purified enzyme stock (2 μL, 4 μL, and 6 μL). Top band indicates (n+1) product. Blank reactions contain all components except enzyme. Samples were analyzed with a 15% TBE-urea gel under denaturing conditions.

FIG. 25 shows a reaction calibration for the controlled incorporation of the reversible terminator 3′-O-azidomethylcarbonate deoxythymidine triphosphate (3′-(0-azidomethyl carbonate)-dTTP) by the S. pombe poly(U) polymerase single mutant H336R. Reactions were incubated for 30 minutes at 37° C. with varying enzyme stock concentrations and nucleotide concentrations. The letter “E” indicates the amount of purified enzyme stock added in μL and the letter “N” indicates the final concentration of nucleotide in mM in the extension reaction. Control reactions contained all components with the exception of nucleotide. After reaction incubation, samples were analyzed with a 15% TBE-urea gel under denaturing conditions. The bottom band indicates unextended starting material and the top band indicates positively extended oligonucleotide.

FIG. 26 shows reaction calibration assessment of purified poly(U) polymerase stock H336R with reversible terminator 3′-O-allyl Adenosine Triphosphate 3′-(O-allyl)-ATP). Gel indicates response of (n+1) extension given increasing input amounts of initiator oligonucleotide. Reactions are supplemented with 1 mM reversible terminator nucleotide and 1 uL of purified enzyme stock. Reactions were incubated for 5 minutes at 37° C. Top band indicates (n+1) product. Blank reactions contain all components except enzyme. Samples were analyzed with a 15% TBE-urea gel under denaturing conditions. This is an example of reaction scalability.

FIG. 27 shows demonstration of controlled enzymatic synthesis using poly(U) polymerase mutant H336R with the reversible terminator with 3′-O-allyl Adenosine Triphosphate (3′-O-allyl-ATP) in bulk solution. Shown here is an (n+5) synthesis in bulk solution. Post-synthesis, reactions were analyzed using a 15% TBE-urea gel under denaturing conditions.

FIG. 28 shows exemplary structures of 3′-reversible terminator nucleotides for enzymatic incorporation. Various examples of protecting groups for the 3′ hydroxy. As labeled, these may be removed through redox chemistry, light, fluoride anions and catalysts.

FIG. 29 shows select 3′ protecting groups where the furanyl ring bears oxygen. The 2′ may be natural ribo, deoxy or various moieties that promote binding, pharmacokinetics, pharmacodynamics, general stability and probe tags.

FIG. 30 shows examples of additional 3′-protecting groups that are irreversible (capping) terminators and esterase sensitive terminators for both non-bridged and bridged nucleoside triphosphates. In non-bridged cases, the 2′-may be natural ribo, deoxy or various moieties that promote binding, pharmacokinetics, pharmacodynamics, general stability and probe tags. Theses 3′-protecting groups can further derivatized with other important moieties such as amino acids, oligonucleotides, or large chemical domains that may confer additional functionality of the 3′-terminus of the synthesized oligonucleotide and may be used as a final, irreversible cap if not sensitive to any known deprotection methods.

FIG. 31 shows an exemplary scheme for the preparation of a 3′ azidomethyl ether for a nucleotide triphosphate, where the 2′ may be natural OH or various modifications such as —F, —OMe, —OCH₂CH₂CH₃ or others which prove beneficial for biological activity of target oligoes or contributing to the broader impacts of science.

FIG. 32 shows an exemplary scheme for the preparation of a 3′ azidomethyl ether for a locked nucleotide triphosphate.

FIG. 33 shows an exemplary scheme for the preparation of a 3′ allyl ether for a nucleotide triphosphate, where the 2′ may be natural OH or various modifications such as F, OMe, OCH₂CH₂CH₃ or others which prove beneficial for biological activity of target oligoes or contributing to other applications.

FIG. 34 shows an exemplary scheme for the preparation of a 3′ azidomethyl ether for a locked nucleotide triphosphate.

### DETAILED DESCRIPTION OF CERTAIN EMBODIMENTS

Described herein are methods for the de novo synthesis of RNA oligonucleotides using enzymatic catalysis. For example, provided herein are methods for the synthesis of RNA oligonucleotides wherein a terminal transferase enzyme (e.g., a poly(N) polymerase) incorporates one or more nucleotides onto an initiator oligonucleotide. For example, provided herein are methods for preparing RNA oligonucleotides wherein a poly(U) polymerase incorporates one or more modified nucleotides onto an initiator oligonucleotide via a terminal transferase.

In one aspect, provided herein are methods wherein modified nucleotides (i.e., 2′- or 3′-modified reversible terminator oligonucleotides) are incorporated that reversibly alter the binding affinity of the polymerase (e.g., a poly(U) polymerase) for the extended initiator oligonucleotide, thereby producing a (n+1) extended RNA oligonucleotide which can be deprotected and then further extended.

In another aspect, provided herein are methods for RNA oligonucleotide synthesis wherein non-hydrolyzable nucleotides are used to control the rate at which a polymerase (e.g., a poly(U) polymerase) incorporates hydrolyzable nucleotides onto an initiator oligonucleotide.

In another aspect, provided herein are methods of ligating two oligonucleotides using a poly(N)polymerase described herein (e.g., a wild-type or mutated poly(U) polymerase described herein) to yield a desired RNA oligonucleotide.

Additionally, RNA oligonucleotides produced by these methods can undergo reverse transcription (RT) to yield complementary DNA (e.g., cDNA) that is amplifiable by a high-fidelity DNA polymerase via the polymerase chain reaction (PCR). Also, provided herein are RNA oligonucleotides and DNA oligonucleotides produced by any method described herein.

Also provided herein are modified nucleotides that are useful in the methods described herein, as well as poly(N) polymerase enzymes (e.g., mutant poly(U) polymerases) that are useful in the methods described herein.

Also provided herein are compositions and kits comprising one or more of the poly(N) polymerases and/or nucleotides described herein. In another aspect, provided herein are reaction mixtures and systems for carrying out the methods described herein.

### RNA Oligonucleotide Synthesis

Provided herein are methods for the synthesis of RNA oligonucleotides wherein a poly(N) polymerase incorporates one or more modified nucleotides onto an initiator oligonucleotide via a terminal transferase (e.g., a poly(N) polymerase). In certain embodiments, provided herein is a method for template-independent synthesis of an RNA oligonucleotide, the method comprising:

(a) providing an initiator oligonucleotide, wherein the initiator oligonucleotide is single-stranded RNA;

(b) providing a poly(N) polymerase;

(c) combining the initiator oligonucleotide, the poly(N) polymerase, and one or more modified nucleotides under conditions sufficient for the addition of at least one modified nucleotide to the 3′ end of the initiator oligonucleotide.

In certain embodiments, the poly(N) polymerase is a poly(U) polymerase. Therefore, in certain embodiments, provided herein is a method for template-independent synthesis of an RNA oligonucleotide, the method comprising:

(a) providing an initiator oligonucleotide, wherein the initiator oligonucleotide is single-stranded RNA;

(b) providing a poly(U) polymerase;

(c) combining the initiator oligonucleotide, the poly(U) polymerase, and one or more modified nucleotides under conditions sufficient for the addition of at least one modified nucleotide to the 3′ end of the initiator oligonucleotide.

Once the one or more modified nucleotides are added to the initiator oligonucleotide, one or more additional nucleotides (modified or unmodified) can be added subsequently in order to synthesize a desired RNA oligonucleotide. Therefore, in certain embodiments, the method further comprises adding one or more natural or modified nucleotides to the 3′ end of the resulting RNA oligonucleotide (i.e., the RNA oligonucleotide formed in step (c)) until a desired RNA sequence is obtained. In certain embodiments, one or more additional modified nucleotides are added. In certain embodiments, the method further comprises:

(d) repeating steps (a)-(c) until a desired RNA sequence is obtained.

### Poly(N) Polymerases

As described herein, the enzyme incorporating one or more nucleotides is an RNA polymerase, such as a poly(N) polymerase. Provided herein are poly(N) polymerases, e.g., mutant (i.e., mutated) poly(U) polymerases, that are useful in the methods described herein.

In certain embodiments, the poly(N) polymerase is a poly(U) polymerase, a poly(A) polymerase, a poly(C) polymerase, or a poly(G) polymerase. The RNA polymerase may be a wild-type polymerase, or a mutant (i.e., mutated), variant, or homolog thereof. In certain embodiments, the poly(N) polymerase is a wild-type polymerase. In certain embodiments, the polymerase is a mutant of a poly(N) polymerase. In certain embodiments, the polymerase is a variant of a poly(N) polymerase. In certain embodiments, a mutant or variant is approximately 60%, 65%, 70%, 75%, 80%, 85%, 90%, 95%, 98%, or 99% identical to the wild-type polymerase. In certain embodiments, the polymerase is a homolog of a poly(N) polymerase.

In certain embodiments, the poly(N) polymerase is a poly(U) polymerase. In certain embodiments, the poly(U) polymerase is wild-type Schizosaccharomyces pombe poly(U) polymerase, or a mutant thereof, or a homolog thereof. In certain embodiments, the poly(U) polymerase is wild-type Schizosaccharomyces pombe poly(U) polymerase. In certain embodiments, the poly(U) polymerase is a mutant of Schizosaccharomyces pombe poly(U) polymerase. In certain embodiments, the poly(U) polymerase is a variant of Schizosaccharomyces pombe poly(U) polymerase. In certain embodiments, the poly(U) polymerase is a homolog of Schizosaccharomyces pombe poly(U) polymerase.

In certain embodiments, the poly(N) polymerase is a poly(A) polymerase. In certain embodiments, the poly(A) polymerase is wild-type Saccharomyces cerevisiae poly(A) polymerase, or a mutant thereof. In certain embodiments, the poly(N) polymerase is wild-type Saccharomyces cerevisiae poly(A) polymerase. In certain embodiments, the poly(N) polymerase is a mutant of Saccharomyces cerevisiae poly(A) polymerase. In certain embodiments, the poly(N) polymerase is a variant of Saccharomyces cerevisiae poly(A) polymerase. In certain embodiments, the poly(N) polymerase is a homolog of Saccharomyces cerevisiae poly(A) polymerase.

### Poly(U) Polymerase Mutants

As described herein, in certain embodiments, the poly(N) polymerase is a mutant of a poly(N) polymerase (i.e., mutated poly(N) polymerase). In certain embodiments, the poly(N) polymerase is an Schizosaccharomyces pombe poly(U) (S. pombe poly(u)) polymerase comprising mutations at one or more positions selected from H336, N171, and T172.

In certain embodiments, the poly(N) polymerase is an Schizosaccharomyces pombe poly(U) (S. pombe poly(u)) polymerase comprising an H336 mutation (i.e., wherein the amino acid H at position 336 is replaced with another amino acid). In certain embodiments, the poly(N) polymerase is an S. pombe poly(u) polymerase comprising an H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W. In certain embodiments, the H336 mutation is the only mutation. In certain embodiments, the poly(N) polymerase comprises one or more addition mutations and is about 60%, 65%, 70%, 75%, 80%, 85%, 90%, 95%, 98%, or 99% identical to SEQ ID NO:3. In certain embodiments, the poly(N) polymerase is identical to SEQ ID NO:3, but includes one H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W.

In certain embodiments, the poly(N) polymerase is an S. pombe poly(u) polymerase comprising an H336R mutation. In certain embodiments, the H336R mutation is the only mutation. In certain embodiments, the poly(N) polymerase comprises one or more addition mutations and is about 60%, 65%, 70%, 75%, 80%, 85%, 90%, 95%, 98%, or 99% identical to SEQ ID NO:3. In certain embodiments, the poly(N) polymerase is identical to SEQ ID NO:3, but includes one mutation: H336R.

In certain embodiments, the poly(N) polymerase is an S. pombe poly(u) polymerase comprising an N171 mutation. In certain embodiments, the poly(N) polymerase is an S. pombe poly(u) polymerase comprising an N171 mutation selected from the group consisting of N171E, N171L, N171Q, N171S, N171M, N171D, N171G, N171C, N171A, N171W, N171T, N171I, N171V, N171P, N171R, N171H, and N171K. In certain embodiments, the N171 mutation is the only mutation. In certain embodiments, the poly(N) polymerase comprises one or more addition mutations and is about 60%, 65%, 70%, 75%, 80%, 85%, 90%, 95%, 98%, or 99% identical to SEQ ID NO:3. In certain embodiments, the poly(N) polymerase is identical to SEQ ID NO:3, but includes one N171 mutation selected from the group consisting of N171E, N171L, N171Q, N171S, N171M, N171D, N171G, N171C, N171A, N171W, N171T, N171I, N171V, N171P, N171R, N171H, and N171K.

In certain embodiments, the poly(N) polymerase is an S. pombe poly(u) polymerase comprising an N171A mutation. In certain embodiments, the N171A mutation is the only mutation. In certain embodiments, the poly(N) polymerase comprises one or more addition mutations and is about 60%, 65%, 70%, 75%, 80%, 85%, 90%, 95%, 98%, or 99% identical to SEQ ID NO:3. In certain embodiments, the poly(N) polymerase is identical to SEQ ID NO:3, but includes one mutation: N171A.

In certain embodiments, the poly(N) polymerase is an S. pombe poly(u) polymerase comprising an N171T mutation. In certain embodiments, the N171T mutation is the only mutation. In certain embodiments, the poly(N) polymerase comprises one or more addition mutations and is about 60%, 65%, 70%, 75%, 80%, 85%, 90%, 95%, 98%, or 99% identical to SEQ ID NO:3. In certain embodiments, the poly(N) polymerase is identical to SEQ ID NO:3, but includes one mutation: N171T.

In certain embodiments, the poly(N) polymerase is an S. pombe poly(u) polymerase comprising an T172 mutation. In certain embodiments, the poly(N) polymerase is an S. pombe poly(u) polymerase comprising an T172 mutation selected from the group consisting of T172E, T172L, T172Q, T172S, T172M, T172D, T172G, T172C, T172A, T172W, T172T, T172I, T172V, T172P, T172R, T172H, and T172K. In certain embodiments, the T172 mutation is the only mutation. In certain embodiments, the poly(N) polymerase comprises one or more addition mutations and is about 60%, 65%, 70%, 75%, 80%, 85%, 90%, 95%, 98%, or 99% identical to SEQ ID NO:3. In certain embodiments, the poly(N) polymerase is identical to SEQ ID NO:3, but includes one T172 mutation selected from the group consisting of T172E, T172L, T172Q, T172S, T172M, T172D, T172G, T172C, T172A, T172W, T172T, T172I, T172V, T172P, T172R, T172H, and T172K.

In certain embodiments, the poly(N) polymerase is an S. pombe poly(u) polymerase comprising H336 and N171 mutations. In certain embodiments, the poly(N) polymerase is an S. pombe poly(u) polymerase comprising an H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W; and an N171 mutation selected from the group consisting of N171E, N171L, N171Q, N171S, N171M, N171D, N171G, N171C, N171A, N171W, N171T, N171I, N171V, N171P, N171R, N171H, and N171K. In certain embodiments, the H336 and N171 mutations are the only mutations. In certain embodiments, the poly(N) polymerase comprises one or more addition mutations and is about 60%, 65%, 70%, 75%, 80%, 85%, 90%, 95%, 98%, or 99% identical to SEQ ID NO:3. In certain embodiments, the poly(N) polymerase is identical to SEQ ID NO:3, but includes one H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W; and one N171 mutation selected from the group consisting of N171E, N171L, N171Q, N171S, N171M, N171D, N171G, N171C, N171A, N171W, N171T, N171I, N171V, N171P, N171R, N171H, and N171K.

In certain embodiments, the poly(N) polymerase is an S. pombe poly(u) polymerase comprising H336R and N171A mutations. In certain embodiments, the H336R and N171A mutations are the only mutations. In certain embodiments, the poly(N) polymerase comprises one or more addition mutations and is about 60%, 65%, 70%, 75%, 80%, 85%, 90%, 95%, 98%, or 99% identical to SEQ ID NO:3. In certain embodiments, the poly(N) polymerase is identical to SEQ ID NO:3, but includes two mutations: H336R and N171A.

In certain embodiments, the poly(N) polymerase is an S. pombe poly(u) polymerase comprising H336R and N171T mutations. In certain embodiments, the H336R and N171T mutations are the only mutations. In certain embodiments, the poly(N) polymerase comprises one or more addition mutations and is about 60%, 65%, 70%, 75%, 80%, 85%, 90%, 95%, 98%, or 99% identical to SEQ ID NO:3. In certain embodiments, the poly(N) polymerase is identical to SEQ ID NO:3, but includes two mutations: H336R and N171T.

In certain embodiments, the poly(N) polymerase is an S. pombe poly(u) polymerase comprising H336 and T172 mutations. In certain embodiments, the poly(N) polymerase is an S. pombe poly(u) polymerase comprising an H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W; and a T172 mutation selected from the group consisting of T172E, T172L, T172Q, T172S, T172M, T172D, T172G, T172C, T172A, T172W, T172T, T172I, T172V, T172P, T172R, T172H, and T172K. In certain embodiments, the H336 and T172 mutations are the only mutations. In certain embodiments, the poly(N) polymerase comprises one or more addition mutations and is about 60%, 65%, 70%, 75%, 80%, 85%, 90%, 95%, 98%, or 99% identical to SEQ ID NO:3. In certain embodiments, the poly(N) polymerase is identical to SEQ ID NO:3, but includes one H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W; and one T172 mutation selected from the group consisting of T172E, T172L, T172Q, T172S, T172M, T172D, T172G, T172C, T172A, T172W, T172T, T172I, T172V, T172P, T172R, T172H, and T172K. In certain embodiments, the H336 mutation is H336R.

In certain embodiments, the poly(N) polymerase is an S. pombe poly(u) polymerase comprising H336, N171, and T172 mutations. In certain embodiments, the poly(N) polymerase is an S. pombe poly(u) polymerase comprising an H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W; an N171 mutation selected from the group consisting of N171E, N171L, N171Q, N171S, N171M, N171D, N171G, N171C, N171A, N171W, N171T, N171I, N171V, N171P, N171R, N171H, and N171K; and a T172 mutation selected from the group consisting of T172E, T172L, T172Q, T172S, T172M, T172D, T172G, T172C, T172A, T172W, T172T, T172I, T172V, T172P, T172R, T172H, and T172K. In certain embodiments, the H336, N171, and T172 mutations are the only mutations. In certain embodiments, the poly(N) polymerase comprises one or more addition mutations and is about 60%, 65%, 70%, 75%, 80%, 85%, 90%, 95%, 98%, or 99% identical to SEQ ID NO:3. In certain embodiments, the poly(N) polymerase is identical to SEQ ID NO:3, but includes one H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W; one N171 mutation selected from the group consisting of N171E, N171L, N171Q, N171S, N171M, N171D, N171G, N171C, N171A, N171W, N171T, N171I, N171V, N171P, N171R, N171H, and N171K; and one T172 mutation selected from the group consisting of T172E, T172L, T172Q, T172S, T172M, T172D, T172G, T172C, T172A, T172W, T172T, T172I, T172V, T172P, T172R, T172H, and T172K. In certain embodiments, the H336 mutation is H336R. In certain embodiments, the N171 mutation is N171A or N171T.

### Modified RNA Nucleotides

As described herein, one or more modified nucleotides can be incorporated into an oligonucleotide in order to synthesize a desired RNA oligonucleotide. Modified nucleotides can be incorporated in order to prepare custom RNA or DNA oligonucleotides. In other embodiments, modified nucleotides can be incorporated to block the incorporation of subsequent nucleotides (i.e., via the use of “reversible terminators” as described herein). Provided herein are modified nucleotides that are useful in the methods described herein, as well as in other applications (e.g., chemical oligonucleotide synthesis, therapeutic applications, etc.)

A “modified nucleotide” is an nucleotide monomer (e.g., comprising a ribose sugar, a phosphate group, and a nucleobase) comprising one or more non-natural modifications. In certain embodiments, for example, the modified nucleotide is the structural equivalent of a naturally occurring RNA or DNA nucleotide (i.e., guanine (G), uracil (U), adenine (A), cytosine (C)) but comprising one or more non-natural modifications. In certain embodiments, the modified nucleotide is the equivalent of a naturally occurring nucleotide, wherein one or more positions are substituted, or wherein one or more substituents or groups are removed or replaced. In certain embodiments, the modified nucleotide comprises a modified sugar, a modified base, a modified phosphate, or any combination thereof. “Modified nucleotides” include the 2′- and 3′-reversible terminator nucleotides described herein.

The following formula is intended to show possible modification sites on an nucleotide. Other modifications are contemplated. In certain embodiments, a modified nucleotide is of the following formula:

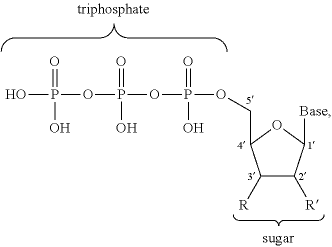

or a salt thereof, wherein:

“Base” (also “B” herein) is a natural or non-natural nucleotide base; and

R and R′ are independently hydrogen or a natural or non-natural sugar substituent.

In certain embodiments, a modified nucleotide is of the following formula:

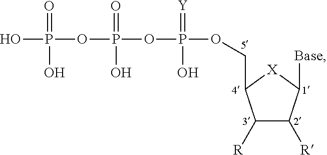

or a salt thereof, wherein:

X is O or S;

Y is O or S;

“Base” (also “B” herein) is a natural or non-natural nucleotide base; and

R and R′ are independently hydrogen or a natural or non-natural sugar substituents.

In certain embodiments, Y is O. In certain embodiments, Y is S. In certain embodiments, X is O. In certain embodiments, X is S.

In certain embodiments, a modified nucleotide is a base-modified nucleotide. “Base-modified” encompasses nucleotides, wherein a G, U, A, or C base is substituted or modified, or wherein a G, U, A, or C base is replaced by a different group (e.g., hypoxanthine).

Non-limiting examples of modified bases include, but are not limited to, 5-methylcytosine, pyridin-4-one, pyridin-2-one, phenyl, pseudouracil, 3-methyl uracil, dihydrouridine, naphthyl, aminophenyl, 5-alkylcytidines, 5-alkyluridines, 5-halouridines, 6-azapyrimidines, 6-alkylpyrimidines, propyne, quesosine, 2-thiouridine, 4-thiouridine, 4-acetyltidine, 5-(carboxyhydroxymethyl)uridine, 5-carboxymethylaminomethyl-2-thiouridine, 5-carboxymethylaminomethyluridine, β-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, 3-D-mannosylqueosine, uridine-5-oxyacetic acid, 2-thiocytidine, and threonine derivatives.

Other non-limiting examples of bases include, but are not limited to, natural or non-natural pyrimidine or purine; and may include, but are not limited to, N¹-methyl-adenine, N⁶-methyl-adenine, 8′-azido-adenine, N,N-dimethyl-adenosine, aminoallyl-adenosine, 5′-methyl-uridine, pseudouridine, N¹-methyl-pseudouridine, 5′-hydroxy-methyl-uridine, 2′-thio-uridine, 4′-thio-uridine, hypoxanthine, xanthine, 5′-methyl-cytidine, 5′-hydroxy-methyl-cytidine, 6′-thio-guanine, and N⁷-methyl-guanine.

In certain embodiments, the base-modified nucleotide is selected from the group consisting of N¹-methyladenosine-5′-triphosphate, N⁶-methyladenosine-5′-triphosphate, N⁶-methyl-2-aminoadenosine-5′-triphosphate, 5-methyluridine-5′-triphosphate, N¹-methylpseudouridine-5′-triphosphate, pseudouridine-5′-triphosphate, 5-hydroxymethyluridine-5′-triphosphate, 5-methylcytidine-5′-triphosphate, 5-hydroxymethylcytidine-5′-triphosphate, N⁷-methylguanosine-triphosphate, 8′-adizoadenisone-5′-triphosphate, inosine 5′-triphosphate, 2-thiouridine-5′-triphosphate, 6-thioguanosine-5′-triphosphate, 4-thiouridine-5′-triphosphate, and xanthosine-5′-triphosphate.

In certain embodiments, the modified nucleotide is a sugar-modified nucleotide. “Sugar-modified” nucleotides encompass nucleotides wherein the ribose or deoxyribose moiety is substituted, or wherein the ribose or deoxyribose is replaced by a different sugar moiety. In certain embodiments, the ribose or deoxyribose is modified (e.g., substituted) at the 1′, 2′, 3′, 4′, and/or 5′ position. In some embodiments, a nucleotide may be modified at the 2′ position. In some embodiments, a nucleotide may be modified at the 3′ position.

In certain embodiments, the 2′ and/or 3′ position of a sugar is substituted with a natural or non-natural “sugar substituent” R or R′. In certain embodiments, R and R′ are independently selected from the group consisting of hydrogen, halogen, —CN, —N02, —N3, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted hydroxyl, optionally substituted amino, or optionally substituted thiol.

In certain embodiments, R and/or R′ are independently —OR^{P}, wherein each instance of R^{P} is independently an oxygen protecting group, optionally substituted acyl, or an amino acid. In certain embodiments, R and/or R′ comprise a reactive moiety for bioconjugation (e.g., click chemistry handle, e.g., azide or alkyne), a fluorophore, catalytic protein, oligonucleotide, or reporting tag.

In some embodiments, the 2′ position of a sugar, e.g., ribose, may be modified with a halogen, e.g., a fluorine group; an alkyl group, e.g., methyl or ethyl group; a methoxy group; an amino group; a thio group; an aminopropyl group; a dimethylaminoethyl; a dimethylaminopropyl group; a dimethylaminoethyloxyethyl group; an azido group; a silyl group; a cyclic alkyl group; or a N-methylacetamido group.

In certain embodiments, the 2′ position of a sugar, e.g., ribose, is modified with a hydroxyl (—OH), hydrogen (—H), fluoro (—F), amine (—NH₃), azido (—N3), thiol (—SH), methoxy (—OCH₃), or methoxyethanol (—OCH₂CH₂OCH₃).

In certain embodiments, the 2′ position may also be substituted with redox-active, fluorogenic or intrinsically fluorescent moieties, natural and non-natural amino acids, peptides, proteins, mono- or oligosaccharides, functional/ligand binding glycans, and polymers or large molecules such as polyethylene glycol (PEG).

In some embodiments, the 3′ position of a sugar, e.g., ribose, may be modified with a halogen, e.g., a fluorine group; an alkyl group, e.g., methyl or ethyl group; a methoxy group; an amino group; a thio group; an aminopropyl group; a dimethylaminoethyl; a dimethylaminopropyl group; a dimethylaminoethyloxyethyl group; an azido group; a silyl group; a cyclic alkyl group; or a N-methylacetamido group.

In certain embodiments, the 3′ position of a sugar, e.g., ribose, is modified with a hydroxyl (—OH), hydrogen (—H), fluoro (—F), amine (—NH₃), azido (—N3), thiol (—SH), methoxy (—OCH₃), or methoxyethanol (—OCH₂CH₂OCH₃).

In certain embodiments, the 3′ position may also be substituted with redox-active, fluorogenic or intrinsically fluorescent moieties, natural and non-natural amino acids, peptides, proteins, mono- or oligosaccharides, functional/ligand binding glycans, and polymers or large molecules such as polyethylene glycol (PEG).

In certain embodiments, the sugar-modified nucleotide is modified at the 2′-position. For example, in certain embodiments, the sugar-modified nucleotide is a 2′-F, 2′-O-alkyl, 2′-amino, or 2′-azido modified nucleotide.

In certain embodiments, the sugar-modified nucleotide is a 2′-F modified nucleotide. In certain embodiments, the sugar-modified nucleotide is selected from the group consisting of 2′-fluoro-2′-deoxyadenosine-5′-triphosphate, 2′-fluoro-2′-deoxycytidine-5′-triphosphate, 2′-fluoro-2′-deoxyguanosine-5′-triphosphate, and 2′-fluoro-2′-deoxyuridine-5′-triphosphate.

In certain embodiments, the sugar-modified nucleotide is a 2′-O-alkyl modified nucleotide. In certain embodiments, the sugar-modified nucleotide is selected from the group consisting of 2′-O-methyladenosine-5′-triphosphate, 2′-O-methylcytidine-5′-triphosphate, 2′-O-methylguanosine-5′-triphosphate, 2′-O-methyluridine-5′-triphosphate, and 2′-O-methylinosine-5′-triphosphate.

In certain embodiments, the sugar-modified nucleotide is a 2′-O-amino modified nucleotide. In certain embodiments, the sugar-modified nucleotide is selected from the group consisting of 2′-amino-2′-deoxycytidine-5′-triphosphate, 2′-amino-2′-deoxyuridine-5′-triphosphate, 2′-amino-2′-deoxyadenosine-5′-triphosphate, and 2′-amino-2′-deoxyguanosine-5′-triphosphate.

In certain embodiments, the sugar-modified nucleotide is a 2′-O-azido modified nucleotide. In certain embodiments, the sugar-modified nucleotide is selected from the group consisting of 2′-azido-2′-deoxycytidine-5′-triphosphate, 2′-azido-2′-deoxyuridine-5′-triphosphate, 2′-azido-2′-deoxyadenosine-5′-triphosphate, and 2′-azido-2′-deoxyguanosine-5′-triphosphate.

In certain embodiments the modified nucleoside triphosphate is an irreversible terminator, also known as a capping nucleotide, such as 3′-O-methyl-NTP, 3′-O-methyl-dNTP, 3′-azido-dNTP, 3′-azido-NTP, 3′-amine-dNTP, 3′-amine-NTP, etc.

In certain embodiments, the sugar-modified nucleotide is a 2′-modified reversible terminator RNA nucleotide (e.g., 2′-O-protected reversible terminator nucleotide). 2′-modified reversible terminator nucleotides are described herein. In certain embodiments, the 2′-modified reversible terminator nucleotide also comprises a modified base moiety.

In certain embodiments, the sugar-modified nucleotide is a 3′-modified reversible terminator RNA nucleotide (e.g., 3′-O-protected reversible terminator nucleotide). 3′-modified reversible terminator nucleotides are described herein. In certain embodiments, the 3′-modified reversible terminator nucleotide also comprises a modified base moiety.

Other modifications to the sugar are contemplated. These modifications include, but not limited to, replacing the ring's oxygen with a sulfur. In certain embodiments, a bridge is introduced between the 2′-carbon and the 4′-carbon (e.g., to limit ring conformation). In some embodiments, an modified nucleotide is a bridged nucleotide, e.g., locked nucleic acid (LNA); a constrained ethyl nucleotide (cEt), or an ethylene bridged nucleic acid (ENA) nucleotide.

In some embodiments, a nucleotide, e.g., a nucleotide, may comprise a modified phosphate group, e.g., a phosphorothioate. Non-limiting examples of modified phosphate groups include phosphorothioates, phosphotriesters, methyl phosphonates, alkyl, heterocyclic, amide, morpholino, peptide nucleic acids (PNA), and other known phosphorus-containing groups. In certain embodiments, the modification is to the alpha (α) phosphate of the triphosphate. In certain embodiments, the nucleotide is an (α) thiophosphonate. In certain embodiments, the modifications to the beta (β) and/or gamma (γ) phosphates of the triphosphate.

In certain embodiments, nucleotide modified with a fluorophore can be used to verify the success of each iterative incorporation event, thereby producing in some embodiments virtually error-free RNA oligonucleotides. In certain embodiments, a modified nucleotide comprises a fluorophore.

Modified nucleotides may comprise more than one modification. For example, a modified nucleotide may comprise a base modification and a sugar modification.

RNA Oligonucleotide Synthesis with Reversible Terminators

Also provided herein are methods of synthesizing RNA oligonucleotides using reversible terminator nucleotides. A “reversible terminator nucleotide” is an nucleotide comprising a non-natural chemical moiety at the 2′- and/or 3′-position that is capable of being removed. After addition of the reversible-terminator nucleotide to the initiator oligonucleotide, the non-natural chemical moiety 2′- and/or 3′-position blocks the incorporation of a second nucleotide, e.g., by interfering with the binding of the oligonucleotide to the polymerase. The non-natural chemical moiety 2′- and/or 3′-position can then be removed, leaving the 3′-position open to the addition of an additional nucleotide. In certain embodiments, the method allows for the controlled addition of one nucleotide at a time, also referred to as “(n+1)” addition. In certain embodiments, the reversible terminator nucleotide is protected at the 2′- and/or 3′-hydroxyl groups (i.e., “2′- and/or 3′-O-protected reversible terminator nucleotides”).

Provided herein are methods for template-independent synthesis of RNA oligonucleotides, the method comprises:

(a) providing an initiator oligonucleotide, wherein the initiator oligonucleotide is single-stranded RNA;

(b) providing a poly(N) polymerase;

(c) combining the initiator oligonucleotide, the poly(N) polymerase, and a reversible terminator nucleotide under conditions sufficient for the addition of the reversible terminator nucleotide to the 3′ end of the initiator oligonucleotide;

(d) deprotecting the RNA oligonucleotide formed in step (c) at the protected position (e.g., 2′ and/or 3′ position) of the reversible terminator nucleotide; and

(e) optionally, repeating steps (a)-(c) until a desired RNA sequence is obtained.

In certain embodiments, the poly(N) polymerase is a poly(U) polymerase. Provided herein is are methods for template-independent synthesis of RNA oligonucleotides, the methods comprising:

(a) providing an initiator oligonucleotide, wherein the initiator oligonucleotide is single-stranded RNA;

(b) providing a poly(U) polymerase;

(c) combining the initiator oligonucleotide, the poly(U) polymerase, and a 2′- and/or 3′-O-protected reversible terminator nucleotide under conditions sufficient for the addition of the 2′- and/or 3′-O-protected reversible terminator nucleotide to the 3′ end of the initiator oligonucleotide;

(d) deprotecting the RNA oligonucleotide formed in step (c) at the protected 2′- and/or 3′-O-position of the 2′- and/or 3′-O-protected reversible terminator nucleotide;

(e) optionally, repeating steps (a)-(c) until a desired RNA sequence is obtained.

As described herein, 2′-O-protected reversible terminator nucleotides can also be used. In certain embodiments, the poly(N) polymerase is a poly(U) polymerase. Provided herein is are methods for template-independent synthesis of RNA oligonucleotides, the methods comprising:

(a) providing an initiator oligonucleotide, wherein the initiator oligonucleotide is single-stranded RNA;

(b) providing a poly(U) polymerase;

(c) combining the initiator oligonucleotide, the poly(U) polymerase, and a 2′-O-protected reversible terminator nucleotide under conditions sufficient for the addition of the 2′-O-protected reversible terminator nucleotide to the 3′ end of the initiator oligonucleotide;

(d) deprotecting the RNA oligonucleotide formed in step (c) at the protected 2′-O-position of the 2′-O-protected reversible terminator nucleotide;

(e) optionally, repeating steps (a)-(c) until a desired RNA sequence is obtained.

As described herein, 3′-O-protected reversible terminator nucleotides can also be used. Provided herein is are methods for template-independent synthesis of RNA oligonucleotides, the methods comprising:

(a) providing an initiator oligonucleotide, wherein the initiator oligonucleotide is single-stranded RNA;

(b) providing a poly(U) polymerase;

(c) combining the initiator oligonucleotide, the poly(U) polymerase, and a 3′-O-protected reversible terminator nucleotide under conditions sufficient for the addition of the 3′-O-protected reversible terminator nucleotide to the 3′ end of the initiator oligonucleotide;

(d) deprotecting the RNA oligonucleotide formed in step (c) at the protected 3′-O-position of the 3′-O-protected reversible terminator nucleotide;

(e) optionally, repeating steps (a)-(c) until a desired RNA sequence is obtained.

Any poly(N) polymerase described herein can be used in the reversible terminator methods described above. In certain embodiments, a mutant poly(U) polymerase described herein is used to incorporate the reversible terminator nucleotide. In certain embodiments, a mutant poly(U) polymerase described herein is used to incorporate a 3′-reversible terminator nucleotide described herein.

An RNA oligonucleotide of any particular sequence can be synthesized using the methods described herein.

### Reversible Terminator RNA Nucleotides

Some of the methods described herein employ reversible terminator RNA oligonucleotides. A “reversible terminator nucleotide” is a modified nucleotide that comprises a non-natural chemical moiety at the 2′- and/or 3′-position that is capable of being removed. In certain embodiments, the reversible terminator nucleotide is protected at the 2′-O- and/or 3′-O-positions with an oxygen protecting group. Also provided herein are new reversible terminator nucleotides (e.g., 2′-modified reversible terminator nucleotides and 3′-modified reversible terminator nucleotides).

In certain embodiments, the 2′-modified reversible terminator nucleotide is protected at the 2′-O-position with an oxygen protecting group (“2′-O-protected reversible terminator nucleotide”). In certain embodiments, the 3′-modified reversible terminator nucleotide is protected at the 3′-O-position with an oxygen protecting group (“3′-O-protected reversible terminator nucleotide”).

For example, in certain embodiments, the reversible terminator nucleotide (i.e., 2′- and/or 3′-O-protected reversible terminator nucleotide) is of the following formula:

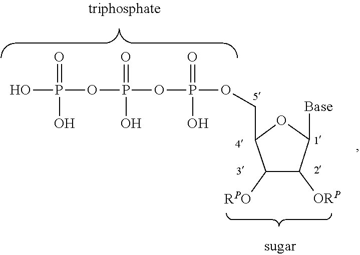

or a salt thereof, wherein:

each instance of R^{P} is hydrogen, an oxygen protecting group, optionally substituted acyl, or an amino acid, or two R^{P} are joined together with the intervening atoms to form optionally substituted heterocyclyl; provided that at least one R^{P} is an oxygen protecting group optionally substituted acyl, or an amino acid; and

“Base” (also “B” herein”) is a natural or non-natural nucleotide (e.g., modified) base. Other portions of the nucleotide can be modified as described above and herein.

For example, in certain embodiments, the reversible terminator nucleotide (i.e., 2′- and/or 3′-O-protected reversible terminator nucleotide) is of the following formula:

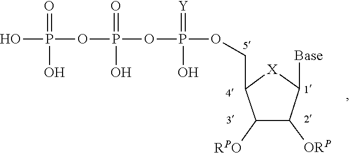

or a salt thereof, wherein:

Y is O or S;

X is O or S;

each instance of R^{P} is hydrogen, an oxygen protecting group, optionally substituted acyl, or an amino acid, or two R^{P} are joined together with the intervening atoms to form optionally substituted heterocyclyl; provided that at least one R^{P} is an oxygen protecting group optionally substituted acyl, or an amino acid; and

“Base” (also “B” herein) is a natural or non-natural nucleotide (e.g., modified) base.

In certain embodiments, a 3′-modified reversible terminator nucleotide (i.e., 3′-O-protected reversible terminator nucleotide) is of the following formula:

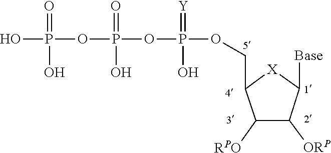

or a salt thereof, wherein:

Y is O or S;

X is O or S;

R^{P} is an oxygen protecting group;

R is hydrogen or a natural or non-natural sugar substituent described herein; and

“Base” (also “B” herein) is a natural or non-natural nucleotide (e.g., modified) base.

Optionally, in certain embodiments, a linking group connects the 2′ carbon to the 4′ carbon (e.g., through the group R′)

In certain embodiments, a 3′-modified reversible terminator nucleotide is a locked or bridged nucleotide. In certain embodiments, a 3′-modified reversible terminator nucleotide (i.e., 3′-O-protected reversible terminator nucleotide) is of the following formula:

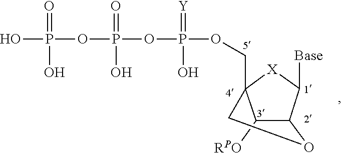

or a salt thereof, wherein:

Y is O or S;

X is O or S;

R^{P} is an oxygen protecting group, optionally substituted acyl, or an amino acid;

R is hydrogen or a natural or non-natural sugar substituent described herein; and

“Base” (also “B” herein) is a natural or non-natural nucleotide (e.g., modified) base.

In certain embodiments, Y is O. In certain embodiments, Y is S. In certain embodiments, X is O. In certain embodiments, X is S.

As described herein, “Base” (also “B” herein) can be any natural or non-naturally occurring nucleobase. Naturally occurring bases include G, U, A, and C. Non-natural (e.g., modified) bases include substituted or modified variants of G, U, A, and C. Non-limiting examples of modified bases include, but are not limited to, 5-methylcytosine, pyridin-4-one, pyridin-2-one, phenyl, pseudouracil, 3-methyl uracil, dihydrouridine, naphthyl, aminophenyl, 5-alkylcytidines, 5-alkyluridines, 5-halouridines, 6-azapyrimidines, 6-alkylpyrimidines, propyne, quesosine, 2-thiouridine, 4-thiouridine, 4-acetyltidine, 5-(carboxyhydroxymethyl)uridine, 5-carboxymethylaminomethyl-2-thiouridine, 5-carboxymethylaminomethyluridine, 3-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, 3-D-mannosylqueosine, uridine-5-oxyacetic acid, 2-thiocytidine, and threonine derivatives.

Other non-limiting examples of bases include, but are not limited to, natural or non-natural pyrimidine or purine; and may include, but are not limited to, N¹-methyl-adenine, N⁶-methyl-adenine, 8′-azido-adenine, N,N-dimethyl-adenosine, aminoallyl-adenosine, 5′-methyl-uridine, pseudouridine, N¹-methyl-pseudouridine, 5′-hydroxy-methyl-uridine, 2′-thio-uridine, 4′-thio-uridine, hypoxanthine, xanthine, 5′-methyl-cytidine, 5′-hydroxy-methyl-cytidine, 6′-thio-guanine, and N⁷-methyl-guanine.

In certain embodiments, the nucleotide sugar is substituted with a natural or non-natural “sugar substituent” R. In certain embodiments, R is hydrogen, halogen, —CN, —NO₂, —N₃, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted hydroxyl, optionally substituted amino, or optionally substituted thiol. In certain embodiments, R is hydrogen. In certain embodiments, R is halogen. In certain embodiments, R is —CN. In certain embodiments, R is —NO₂. In certain embodiments, R is —N₃. In certain embodiments, R is optionally substituted alkyl. In certain embodiments, R is optionally substituted alkenyl. In certain embodiments, R is optionally substituted alkynyl. In certain embodiments, R is optionally substituted aryl. In certain embodiments, R is optionally substituted heteroaryl. In certain embodiments, R is optionally substituted carbocyclyl. In certain embodiments, R is optionally substituted heterocyclyl. In certain embodiments, R is optionally substituted acyl. In certain embodiments, R is optionally substituted hydroxyl. In certain embodiments, R is optionally substituted amino. In certain embodiments, R is optionally substituted thiol.

In certain embodiments, R is —OR^{P}, wherein R^{P} is an oxygen protecting group, optionally substituted acyl, or an amino acid.

In certain embodiments, R comprises a reactive moiety for bioconjugation (e.g., click chemistry handle, e.g., azide or alkyne), a fluorophore, catalytic protein, oligonucleotide, or reporting tag.

In some embodiments, R is halogen, e.g., a fluorine group; an alkyl group, e.g., methyl or ethyl group; a methoxy group; an amino group; a thio group; an aminopropyl group; a dimethylaminoethyl; a dimethylaminopropyl group; a dimethylaminoethyloxyethyl group; an azido group; a silyl group; a cyclic alkyl group; or a N-methylacetamido group.

In certain embodiments, R is hydroxyl (—OH), hydrogen (—H), fluoro (—F), amine (—NH₃), azido (—N₃), thiol (—SH), methoxy (—OCH₃), or methoxyethanol (—OCH₂CH₂OCH₃).

In certain embodiments, R comprises redox-active, fluorogenic or intrinsically fluorescent moiety, natural and non-natural amino acids, peptides, proteins, mono- or oligosaccharides, functional/ligand binding glycans, or polymers or large molecules such as polyethylene glycol (PEG).

As defined herein, each R^{P} is independently an oxygen protecting group, optionally substituted acyl, or an amino acid. In certain embodiments, R^{P} is an oxygen protecting group. In certain embodiments, R^{P} is optionally substituted acyl. In certain embodiments, R^{P} is an amino acid. In certain embodiments, R^{P} is an oxygen protecting group, optionally substituted acyl, or an amino acid that can be cleaved by an esterase.

In certain embodiments, the reversible terminator nucleotide is capable of being deprotected under photochemical conditions. Therefore, the reversible terminator RNA oligonucleotide, in certain embodiments, is protected at the 2′-O- and/or 3′-O-positions with a photolabile oxygen protecting group. In certain embodiments, a 2′-modified reversible terminator nucleotide is protected at the 2′-O position with a photolabile protecting group. In certain embodiments, a 3′-modified reversible terminator nucleotide is protected at the 3′-O position with a photolabile protecting group.

In certain embodiments a 2′- or 3′-O-protecting group (e.g., R^{P}) is of one of the following formulae:

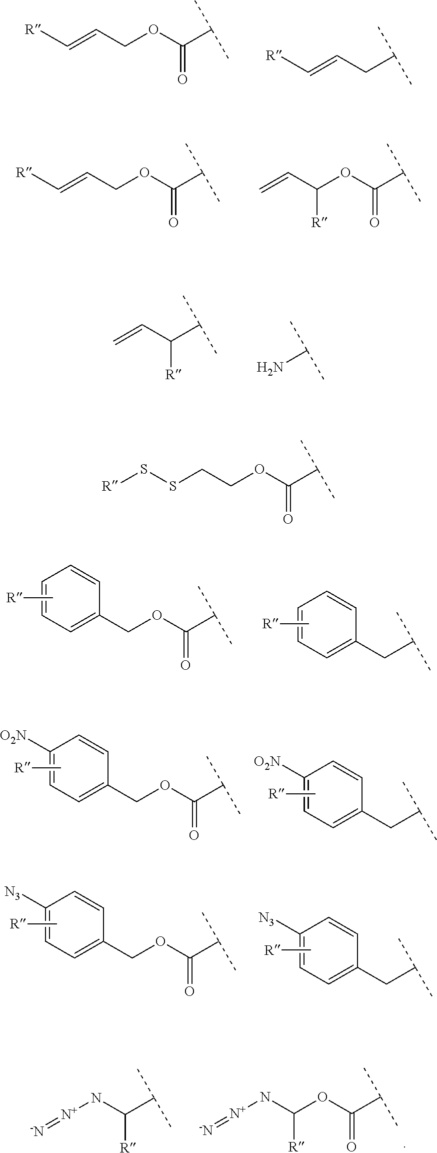

In certain embodiments a 2′- or 3′-O-protecting group (e.g., R^{P}) is of one of the following formulae:

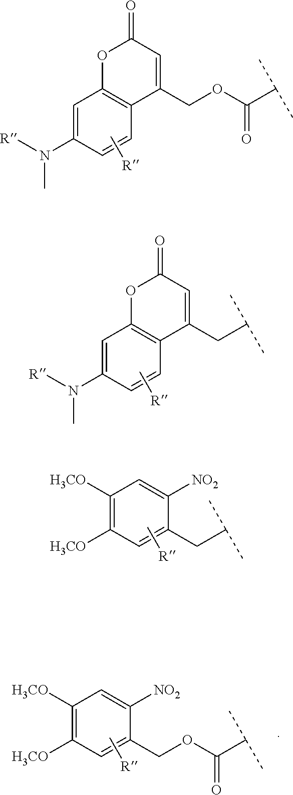

In certain embodiments a 2′- or 3′-O-protecting group (e.g., R^{P}) is of one of the following formulae:

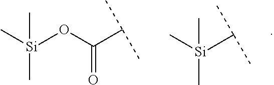

In certain embodiments a 2′- or 3′-O-protecting group (e.g., R^{P}) is of the following formulae:

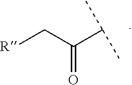

In certain embodiments a 2′- or 3′-O-protecting group (e.g., R^{P}) is an amino acid of the following formula:

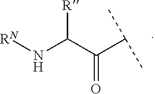

In certain embodiments, each instance of R^{P} is independently alkyl, silyl, allyl, azidomethyl, benzyl, coumarinyl, or carbonate.

In certain embodiments, a 2′-modified reversible terminator nucleotide is a 2′-O-alkyl, 2′-O-silyl, 2′-O-allyl, 2′-O-azidomethyl, 2′-O-benzyl, 2′-O-coumarinyl, or a 2′-O-carbonate modified nucleotide. In certain embodiments, the 2′-modified reversible terminator nucleotide is a 2′-O-carbonate modified nucleotide selected from 2′-O-allyloxycarbonyl and 2′-O-(2-oxo-2H-chromen-4-yl)methyloxycarbonyl.

In certain embodiments, the 2′-O-protected reversible terminator is a 2′-O-allyl-NTP or 2′-O-azidomethyl-NTP.

In certain embodiments, a 3′-modified reversible terminator nucleotide is a 3′-O-alkyl, 3′-O-silyl, 3′-O-allyl, 3′-O-azidomethyl, 3′-O-benzyl, 3′-O-coumarinyl, or a 3′-O-carbonate modified nucleotide. In certain embodiments, the 3′-modified reversible terminator nucleotide is a 3′-O-carbonate modified nucleotide selected from 3′-O-allyloxycarbonyl and 3′-O-(2-oxo-2H-chromen-4-yl)methyloxycarbonyl.

In certain embodiments, the 3′-O-protected reversible terminator is a 3′-O-allyl-NTP, 3′-O-azidomethyl-NTP, 3′-O-allyl carbonate-NTP, 3′-O-allyl carbonate-dNTP, 3′-O-azidomethyl carbonate-NTP, or 3′-O-azidomethyl carbonate-dNTP.

In certain embodiments, the 3′-O-protected reversible terminator is a 3′-O-allyl-NTP, 3′-(O-allyl-carbonate)-dNTP (e.g., 3′-(O-allyl-carbonate)-dATP, etc.), 3′-(O-azidomethyl carbonate)-dNTP, 3′-(O-acetate)-dNTP, 3′-(O-acyl amino acids)-dNTP, 3′-(O-3-methylcoumarin)-dNTP, 3′-(O-(4-methylcoumarin carbonate)-dNTP, 3′-(O-(2-nitrobenzyl)-dNTP, 3′-(O-(2-nitrobenzyl carbonate)-dNTP, 3′-(O-TMS)-dNTP, or 3′-(O-Teoc)-dNTP.

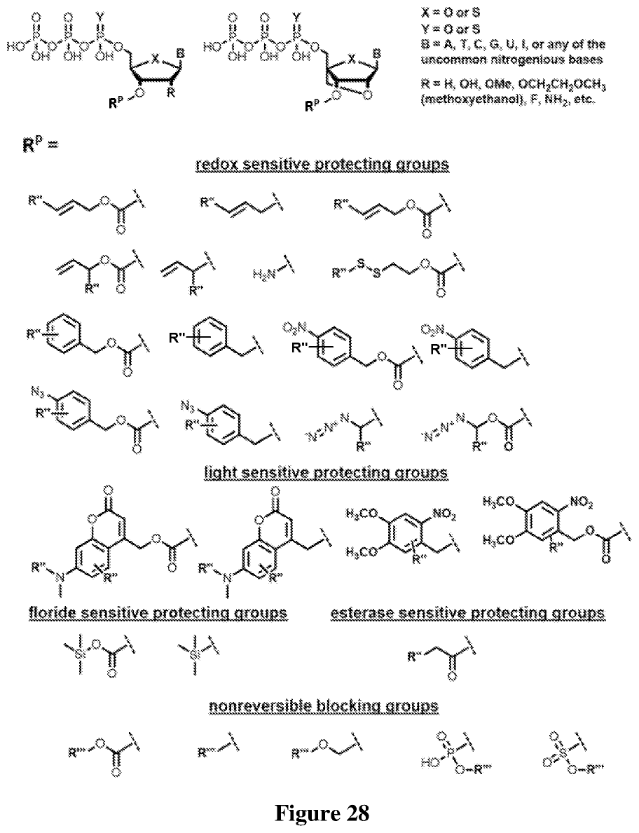

Other certain embodiments of reversible terminator nucleotides, including certain embodiments of 3′-protected nucleotides, are shown in FIGS. 28-34.

As described herein, reversible terminator oligonucleotides may be protected with oxygen protecting groups (e.g, R^{P} groups). Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference. Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, an oxygen protecting group is silyl. In certain embodiments, an oxygen protecting group is t-butyldiphenylsilyl (TBDPS), t-butyldimethylsilyl (TBDMS), triisoproylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv).

In certain embodiments, the 3′-reversible terminator is a 3′-O-amino acid (e.g., comprising any standard or non-standard amino acid). In certain embodiments, the amino acid can be removed using an esterase.

As generally defined herein, R″ is hydrogen, halogen, —CN, —NO₂, —N₃, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted hydroxyl, optionally substituted amino, or optionally substituted thiol. In certain embodiments, R″ is hydrogen. In certain embodiments, R″ is halogen. In certain embodiments, R″ is —CN. In certain embodiments, R″ is —NO₂. In certain embodiments, R″ is —N₃. In certain embodiments, R″ is optionally substituted alkyl. In certain embodiments, R″ is optionally substituted alkenyl. In certain embodiments, R″ is optionally substituted alkynyl. In certain embodiments, R″ is optionally substituted aryl. In certain embodiments, R″ is optionally substituted heteroaryl. In certain embodiments, R″ is optionally substituted carbocyclyl. In certain embodiments, R″ is optionally substituted heterocyclyl. In certain embodiments, R″ is optionally substituted acyl. In certain embodiments, R″ is optionally substituted hydroxyl. In certain embodiments, R″ is optionally substituted amino. In certain embodiments, R″ is optionally substituted thiol.

In certain embodiments, R″ comprises a reactive moiety for bioconjugation (e.g., click chemistry handle, e.g., azide or alkyne), a fluorophore, catalytic protein, oligonucleotide, or reporting tag.

As generally defined herein, R′″ is hydrogen, halogen, —CN, —NO₂, —N₃, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted hydroxyl, optionally substituted amino, optionally substituted thiol, or an oxygen protecting group. In certain embodiments, R′″ is hydrogen. In certain embodiments, R′″ is halogen. In certain embodiments, R′″ is —CN. In certain embodiments, R′″ is —NO₂. In certain embodiments, R′″ is —N₃. In certain embodiments, R′″ is optionally substituted alkyl. In certain embodiments, R′″ is optionally substituted alkenyl. In certain embodiments, R′″ is optionally substituted alkynyl. In certain embodiments, R′″ is optionally substituted aryl. In certain embodiments, R′″ is optionally substituted heteroaryl. In certain embodiments, R′″ is optionally substituted carbocyclyl. In certain embodiments, R′″ is optionally substituted heterocyclyl. In certain embodiments, R′″ is optionally substituted acyl. In certain embodiments, R′″ is optionally substituted hydroxyl. In certain embodiments, R′″ is optionally substituted amino. In certain embodiments, R′″ is optionally substituted thiol.

In certain embodiments, R′″ comprises a reactive moiety for bioconjugation (e.g., click chemistry handle, e.g., azide or alkyne), a fluorophore, catalytic protein, oligonucleotide, or reporting tag.

As generally defined herein, R^{N} is hydrogen, optionally substituted alkyl, optionally substituted acyl, or a nitrogen protecting group. In certain embodiments, R^{N} is hydrogen. In certain embodiments, R^{N} is optionally substituted alkyl. In certain embodiments, R^{N} is optionally substituted acyl. In certain embodiments, R^{N} is a nitrogen protecting group.

In certain embodiments, R^{N} comprises a reactive moiety for bioconjugation (e.g., click chemistry handle, e.g., azide or alkyne), a fluorophore, catalytic protein, oligonucleotide, or reporting tag.

RNA Oligonucleotide Synthesis with Non-Hydrolyzable RNA Nucleotides

Also provided herein are methods for RNA oligonucleotide synthesis employing non-hydrolyzable nucleotides. As described herein, the rate at which a polymerase can incorporate nucleotides (i.e., hydrolyzable nucleotides) at the 3′-terminus of an initiator oligonucleotide can be controlled by introducing a non-hydrolyzable nucleotide that competes for the enzyme's active site. The non-hydrolyzable nucleotide is not incorporated, and the rate of incorporation of the hydrolyzable nucleotide is directly impacted by the ratio of the hydrolyzable nucleotide and the non-hydrolyzable nucleotides through competitive inhibition. Ultimately, the number of incorporations of an nucleotide is determined by the concentration of a non-hydrolyzable nucleotide in the reaction mixture. After a poly(N) polymerase incorporates one or more nucleotides via terminal transferase, the process can be repeated in one or more in iterative steps, optionally with different nucleotides, until a desires RNA oligonucleotide sequence is obtained.

Provided herein are methods for template-independent synthesis of RNA oligonucleotides, the method comprising:

(a) providing an initiator oligonucleotide, wherein the initiator oligonucleotide is single-stranded RNA;

(b) providing a poly(N) polymerase;

(c) combining the initiator oligonucleotide, the poly(N) polymerase, one or more nucleotides, and one or more non-hydrolyzable nucleotides under conditions sufficient for addition of at least one hydrolyzable nucleotide to the 3′ end of the initiator oligonucleotide, wherein the concentration of the non-hydrolyzable nucleotides is sufficient to inhibit the rate of addition of the one or more nucleotides by the poly(N) polymerase.

As described herein, the poly(N) polymerase is, in certain embodiments, a poly(U) polymerase. Provided herein are methods for template-independent synthesis of RNA oligonucleotides, the method comprising:

(a) providing an initiator oligonucleotide, wherein the initiator oligonucleotide is single-stranded RNA;

(b) providing a poly(U) polymerase;

(c) combining the initiator oligonucleotide, the poly(U) polymerase, one or more nucleotides, and one or more non-hydrolyzable nucleotides under conditions sufficient for addition of at least one hydrolyzable nucleotide to the 3′ end of the initiator oligonucleotide, wherein the concentration of the non-hydrolyzable nucleotides is sufficient to inhibit the rate of addition of the one or more nucleotides by the poly(U) polymerase.

In certain embodiments, the concentration of non-hydrolyzable nucleotide is such that 1-100 of the nucleotides are incorporated. In certain embodiments, the concentration of non-hydrolyzable nucleotide is such that 1-50 of the nucleotides are incorporated. In certain embodiments, the concentration of non-hydrolyzable nucleotide is such that 1-20 of the nucleotides are incorporated. In certain embodiments, the concentration of non-hydrolyzable nucleotide is such that less than 100, less than 90, less than 80, less than 70, less than 60, less than 50, less than 40, less than 30, less than 20, less than 10, less than 5, less than 4, less than 3, or less than 2 of the hydrolyzable nucleotides are incorporated.

Once the one or more nucleotides are added to the initiator oligonucleotide, one or more additional nucleotides can be added subsequently in order to synthesize a desired RNA oligonucleotide. Therefore, in certain embodiments, the method further comprises adding one or more natural or modified nucleotides to the 3′ end of the resulting RNA oligonucleotide (i.e., the RNA oligonucleotide formed in step (c)) until a desired RNA sequence is obtained. In certain embodiments, the method further comprises:

(d) repeating steps (a)-(c) until a desired RNA sequence is obtained.

### Non-Hydrolyzable RNA Nucleotides

Methods provided herein employ non-hydrolyzable nucleotides. “Non-hydrolyzable” nucleotides are nucleotides capable of binding to an RNA polymerase, but incapable of undergoing the enzyme-catalyzed addition (i.e., terminal transferase reaction) to an initiator oligonucleotide (e.g., to the 3′ end of the initiator oligonucleotide). In certain embodiments, the non-hydrolyzable nucleotide is a phosphate-modified nucleotide (i.e., comprises a modified triphosphate group). Also provided herein are non-hydrolyzable nucleotides useful in the methods described herein.

For example, in certain embodiments, a non-hydrolyzable nucleotide is of the following formula:

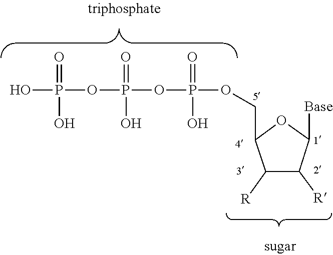

or a salt thereof, wherein:

each Y is independently —O—, —NR^{N}—, —C(R^{C})₂—, or —S—; provided that at least one Y is not —O—;

R and R′ are independently hydrogen or sugar substituents as defined herein;

“Base” is a natural or non-natural (e.g., modified) nucleotide base as defined herein;

R^{N} is hydrogen, optionally substituted alkyl, or a nitrogen protecting group;

and each instance of R^{C} is independently hydrogen, halogen, or optionally substituted alkyl. In certain embodiments, —NR^{N}— is —NH—. In certain embodiments, —C(R^{C})₂— is —CH₂—.

In certain embodiments, the non-hydrolyzable nucleotide comprises a modified triphosphate group. In certain embodiments, the non-hydrolyzable nucleotide is selected from the group consisting of uridine-5′-[(α,β)-imido]triphosphate, adenosine-5′-[(α,β)-imido]triphosphate, guanosine-5′-[(α,β)-methyleno]triphosphate, cytidine-5′-[(α,β)-methyleno]triphosphate, adenosine-5′-[(β,γ)-imido]triphosphate, guanosine-5′-[(β,γ)-imido]triphosphate, and uridine-5′-[(β,γ)-imido]triphosphate. The triphosphate group may comprise any other modifications.

In certain embodiments, the non-hydrolyzable nucleotide is a 3′-modified nucleotide. In certain embodiments, the non-hydrolyzable nucleotide is selected from the group consisting of 3′-O-methyladenosine-5′-triphosphate and 3′-O-methyluridine-5′-triphosphate.

The non-hydrolyzable nucleotide may further comprise any other nucleotide modifications described above and herein.

### RNA Oligonucleotide Synthesis Reactions

The terminal transferase reactions described herein (i.e., step (c) of any of the methods described herein) are carried out in the presence of a polymerase enzyme (e.g., a poly(N) polymerase). In certain embodiments, step (c) is carried out in the presence of one or more additional enzymes. In certain embodiments, step (c) is carried out in the presence of a mixture of two or more different enzymes. The mixture of enzymes may comprise more than one distinct poly(N) polymerases (e.g., 2 or 3 different poly(N) polymerases). The mixture of poly(N) polymerase enzymes may include both wild-type and mutates poly(N) polymerases (e.g., mutated poly(U) polymerases provided herein).

In certain embodiments, step (c) is carried out in the presence of one or more additional phosphatases in addition to the poly(N) polymerase. In certain embodiments, step (c) is carried out in the presence of a yeast inorganic pyrophosphatase (PPI-ase) in addition to the poly(N) polymerase.

In certain embodiments, the terminal transferase reaction in step (c) is carried out in the presence of one or more additional additives. In certain embodiments, step (c) is carried out in the presence of a crowding agent. In certain embodiments, the crowing agent is polyethylene glycol (PEG) or Ficoll. In certain embodiments, the crowding agent is polyethylene glycol (PEG). In certain embodiments, step (c) is carried out in the presence of an RNase inhibitor. In certain embodiments, step (c) is carried out in the presence of a non-hydrolyzable nucleotide.

### Initiator Oligonucleotides

The methods described herein use initiator oligonucleotides. The initiator oligonucleotides may be of any sequence and can be any number of nucleotides in length. In certain embodiments, the initiator oligonucleotide is 20 nucleotides or less in length. In certain embodiments, the initiator oligonucleotide is 5-20 nucleotides in length. In certain embodiments, the initiator oligonucleotide is more than 20 nucleotides in length.

In certain embodiments, the initiator oligonucleotide is a poly-rN oligonucleotide. In certain embodiments, the initiator oligonucleotide is a poly-rU, poly-rC, poly-rG, or poly-rA.

The initiator oligonucleotide may also be covalently linked to a solid support. In certain embodiments, the oligonucleotide is cleaved from the solid support after a desired RNA oligonucleotide sequence is obtained. Therefore, in certain embodiments, the initiator oligonucleotide is covalently linked to a solid support through a cleavable linker.

The initiator oligonucleotides can comprise other modification such as fluorophores. In certain embodiments, the initiator oligonucleotide comprises a 5′-fluorophore. In certain embodiments, the fluorophore is Cy5 or FAM. The initiator oligonucleotide may also comprise one or more additional functional groups or handles for bioconjugation. In certain embodiments, the initiator oligonucleotide is functionalized with biotin.

In certain embodiments, the initiator oligonucleotide comprises a 5′-phosphate (e.g., 5′-mono-, di-, or triphosphate). In certain embodiments, the initiator oligonucleotide comprises a 5′-monophosphate. In certain embodiments, the initiator oligonucleotide comprises a 5′-diphosphate. In certain embodiments, the initiator oligonucleotide comprises a 5′-triphosphate.

In certain embodiments, the initiator oligonucleotide comprises a 5′-capping group (i.e., 5′ cap).

In certain embodiments, the 5′ cap can be a mono-nucleotide (1-nt), di-nucleotide (2-nt), tri-nucleotide (3-nt), or N-nucleotide (i.e., of any oligonucleotide length that would be useful). The 5′ cap may also comprise a combination of one or more natural and non-natural (e.g., modified) nucleoside bases, including those described herein.

In certain embodiments, the 5′ cap is a guanine cap. In certain embodiments, the 5′ cap is a 7-methylguanylate cap (m⁷G). A In certain embodiments, the guanine or m⁷G cap includes a guanine nucleotide connected to the oligonucleotide via a 5′ to 5′ triphosphate linkage. In certain embodiments, the 5′ cap includes methylation of the 2′ hydroxy-groups of the first and/or second 2 ribose sugars of the 5′ end of the oligonucleotide.

In certain embodiments, the 5′-cap is a 5′-trimethylguanosine cap or a 5′-monomethylphosphate cap. In other embodiments, the 5′ cap is a NAD⁺, NADH, or 3′-dephospho-coenzyme A cap.

In certain embodiments, the initiator oligonucleotide comprises a primer site for reverse transcription of the synthesized RNA oligonucleotide. In certain embodiments, the initiator oligonucleotide comprises a primer site for PCR amplification.

Splicing RNA Fragments Together with 5′-Triphosphorylated Oligonucleotides

In certain embodiments, the methods provided herein can be applied to the splicing of oligonucleotide fragments together (i.e., ligation) using a 5′-triphosphate group by a template-independent polymerase to create a long RNA (e.g., >100-nt in length) molecule.

A 5′-triphosphate oligonucleotide, either synthesized as an initiator oligonucleotide or as the product of controlled, template-independent enzymatic synthesis (e.g., a method described herein), can be a substrate of polymerase such as poly(U) polymerase or mutated variant thereof (e.g., a mutated variant described herein). In certain embodiments, the poly(U) polymerase or mutated variant thereof is accepting of large 3′-modifications. In some instances, the 3′-modification is a series of nucleic acids (i.e., oligonucleotide) instead of a single nucleoside triphosphate or a protecting group.

Thus, provided herein are methods for the synthesis of RNA oligonucleotides, the methods comprising:

(a) providing a first oligonucleotide, wherein the first oligonucleotide comprises a 5′-triphosphate group;

(b) providing a second oligonucleotide;

(c) providing a poly(U) polymerase;

(d) combining the first and second oligonucleotides and the poly(U) polymerase under conditions sufficient for the ligation of the first oligonucleotide to the 3′ end of the second oligonucleotide.

In certain embodiments, the second oligonucleotide is a 3′-OH oligonucleotide.

In certain embodiments, the 5′-triphosphate oligonucleotide is modified to include phosphorothioate at the alpha (α)-phosphate.

In certain embodiments, the first oligonucleotide (e.g., 5′-triphosphorylated oligonucleotide includes one or more modifications to the nucleobases, sugars, or backbone of the oligonucleotide. In certain embodiments, the second oligonucleotide includes one or more modifications to the nucleobases, sugars, or backbone of the oligonucleotide.

In certain embodiments, template-independent ligations occur in reaction conditions that enhance ligation activity, such as the addition of crowding agents, etc. as described herein.

### Reverse Transcription and Amplification of RNA Oligonucleotides

The methods provided herein can be applied to the synthesis of DNA oligonucleotides. After a desired RNA oligonucleotide is obtained via a method described herein, one or more additional steps of reverse transcription and/or amplification can be carried out to yield DNA (e.g., cDNA, ssDNA, double stranded DNA). The end result is a method for controlled, template-independent synthesis of DNA oligonucleotides.

Therefore, in certain embodiments, a method provided herein further comprises a step of:

(f) performing reverse transcription on the resulting RNA oligonucleotide using a reverse transcription priming site, primer, and a reverse transcriptase enzyme to produce a complementary single-stranded DNA oligonucleotide. In certain embodiments, the reverse transcriptase enzyme is a high-fidelity reverse transcriptase enzyme.

In certain embodiments, a method provided herein further comprises a step of:

(g) amplifying the complementary single-stranded DNA oligonucleotide or cDNA produced from synthesized RNA oligonucleotide via reverse transcription in step (f) with a DNA polymerase to be produce double-stranded DNA. In certain embodiments, the DNA polymerase is a high-fidelity DNA polymerase.

### EXAMPLES

### Introduction

Oligonucleotide-based therapeutics are an emerging modality in the rationally-designed and personalized, postgenomic era of medicine (Khvorova et al. 2017). Comprised of short sequences of natural and/or non-natural, modified nucleic acid building blocks, oligonucleotide therapeutics can be specifically tailored to affect a target with maximum efficacy while retaining an optimal pharmacokinetic profile (Deleavey et al. 2012). This is largely defined by the chemical and structural architecture of the oligonucleotide therapeutic, which can include a combination of carefully chosen modifications to the sugar rings, nucleobases, and phosphate backbone as well as the overall three-dimensional structure of the oligonucleotide (Cummins et al. 1995, Eckstein 2014, Watts et al. 2008, Wilson et al. 2006). Both chemical composition and the sequence in which the nucleic acid building blocks are assembled confer the global properties of the oligonucleotide therapeutic; a slight rearrangement or different chemical moiety could potentially improve its therapeutic ability (Khvorova et al. 2017, Koch et al. 2014, Bohr et al. 2017). This is a clear a key advantage over traditional small molecule therapeutics where a major redesign may be necessary for optimization. While there is a diverse array of successful oligonucleotide therapeutics classes, such as short (<50-nt) antisense oligonucleotides (ASOs) (Goyal et al. 2018, Uhlmann et al. 1990), short-interfering RNA (siRNA) (Dana et al. 2017), microRNA (miRNA) (Rupaimoole et al. 2017), etc., as well as longer (>100-nt) messenger RNA (mRNA) (Pardi et al. 2018) and long-noncoding RNA (lncRNA) (Arun et al. 2018), one unifying theme among each is that their production, especially at large scales, is vastly limited by the current state oligonucleotide synthesis technology (Ma et al. 2012).

The synthesis of DNA and RNA oligonucleotides using the phosphoramidite chemistry has been a staple of scientific research since the 1970s (Beaucage et al. 1992). The phosphoramidite chemistry is exceedingly reliable and inexpensive for the synthesis of short, uncomplicated DNA oligonucleotides comprised of the four natural nucleobases. However, aside from the advent of massively parallelized synthesis and automation technology, there has been only minor, incremental improvements to the core methodologies of phosphoramidite-based oligonucleotide synthesis (Beaucage et al. 1992, Kosuri et al. 2014). This is especially true for the chemical synthesis of RNA and heavily modified oligonucleotides, which is still very costly, low-yielding, and often requires multiple purifications post-synthesis that greatly increases the lead-time to isolate appreciable quantities of the desired product (Baronti et al. 2018). Furthermore, the phosphoramidite chemistry is not particularly conducive to a large repertoire of chemical modifications, a necessity for many current oligonucleotide therapeutics (Khvorova et al. 2017), as organic solvents and harsh conditions complicates synthesis schemes by requiring additional protecting groups for labile moieties that may confer unique properties onto the oligonucleotide for delivery or ligand binding purposes, for example (Baronti et al. 2018). In vitro transcription (IVT) strategies have ameliorated some of these limitations; particularly the production of long RNA oligonucleotides (>120-nt), which is currently impossible with the phosphoramidite chemistry (Pardi et al. 2018, Milligan et al. 1987, Sahin et al. 2014). However, IVT does not allow for site-specific labeling of the oligonucleotide, requiring the user to swap out particular bases in addition to using a DNA template for proper enzymatic catalysis. Unfortunately, the combination of high-costs, difficult synthesis, and inaccessibility to diverse nucleic acid building blocks has stifled researchers from developing innovative oligonucleotide therapeutics to combat debilitating diseases. One potential solution to address the aforementioned limitations of oligonucleotide synthesis and oligonucleotide-based therapeutic development is to completely circumvent the use of the phosphoramidite chemistry. Currently, there is great interest in utilizing a class of polymerases known as nucleotidyl transferases, which catalyze the addition of a nucleoside monophosphate to the 3′-end of a short initiator sequence, to synthesize oligonucleotides de novo (Perkel 2019, Pratt et al., 2008). Many nucleotidyl transferases do not require the use of a template sequence and their reactions can be carried out under aqueous conditions, avoiding many of the negative aspects of chemical oligonucleotide synthesis including nucleobase depurination, unwanted insertions or deletions, and the accumulation of irreversibly capped truncation products. Some notable nucleotidyl transferases capable of template-independent de novo oligonucleotide synthesis include, but are not limited to, Terminal deoxynucleotidyl Transferase (TdT) (Motea et al. 2010), Cid1 poly(U) polymerase (PuP) (Munoz-Tello et al. 2012), poly(A) polymerase (PaP) (Balbo et al. 2007), poly(G) polymerase (PgP), poly(C) polymerase (PcP), CCD-adding enzyme (Cho et al. 2007), polymerase Mu (μ) (Dominguez et al. 2000), and polymerase Theta (Θ) (Thomas et al. 2019). Of the aforementioned nucleotidyl transferases, only terminal deoxynucleotidyl transferase has been used in a successful demonstration of enzymatic oligonucleotide synthesis (Palluk et al. 2019). However, only applications in DNA data storage are so far possible as terminal deoxynucleotidyl transferase is difficult to control, has a strong preference for natural deoxynucleoside triphosphates, and is exceedingly biases toward certain nucleobases and initiator combinations over others—attributes that can be computationally correct post-synthesis to retrieve stored data (Ceze et al. 2019, Anavy et al. 2019, Lee et al. 2019). Thus, the development of an enzymatic oligonucleotide synthesis platform that can (1) extend the growing sequence by a single base (n+1) with a reversible blocked modified nucleoside triphosphate, (2) incorporate an array of modified nucleoside triphosphates that confer therapeutic or other value to the oligonucleotide, and (3) be scaled to industrially relevant outputs is of great importance.

### Controlled Enzymatic Synthesis of RNA Oligonucleotides

Several methods for the controlled de novo synthesis of RNA oligonucleotides via enzymatic catalysis have been developed. Engineered and wild-type polymerases with the ability to efficiently incorporate natural and modified ribonucleotide triphosphates (rNTPs) without a template sequence can be used to iteratively add nucleotides to the 3′-OH of an initiator oligonucleotide sequence. Their addition can be through either single or multiple incorporation events. The biologically compatible reaction conditions needed for enzyme functionality greatly reduces the susceptibility of RNA oligonucleotides to degradation that is normally associated with chemical synthesis. These methods can be integrated into a microfluidic or array-based format to synthesize many RNA oligonucleotides in parallel with high cost efficiency. An RNA oligonucleotide synthesized with this method can be produced with a low error rate and will be biologically compatible for downstream biotechnological applications.

The DNA/RNA-directed polymerase, polymerase μ, and the RNA-directed polymerases, poly(A) polymerase (PAP) and poly(U) polymerases are three examples of polymerases that are compatible for the aforementioned RNA synthesis schemes. However, any other polymerase or enzyme with the capacity to add nucleotides to the 3′-terminus of an initiator oligonucleotide without the need of a template sequence could be used, such as CCA-adding enzymes. This includes possible functional mutants that display a similar or increased capacity for controlled de novo RNA synthesis.

Some possible applications of this invention include the following: (1) the cost-efficient and high-fidelity de novo synthesis of RNA oligonucleotides longer than 100-nt, (2) synthesized RNA oligonucleotides can be used as a cheap and high-quality source of biological material such as: synthetic transfer RNA, ribosomal RNA, self-folding RNA structures, novel ribozymes, protein binding complexes, RNA therapeutics, CRISPR/Cas9 Guide RNA, and RNA sequencing probes (such as padlock probes for in situ sequencing), (3) the production of useful, PCR amplifiable, DNA oligonucleotides or gene sequences via conversion by reverse transcription, and (4) enzymes used for RNA synthesis like Pol(p) (a DNA/RNA-directed polymerase) are additionally candidates for controlled enzymatic synthesis of DNA oligonucleotides or gene sequences under biologically compatible reaction conditions.

Controlling rNTP Incorporation Speed with Impeding Reaction Conditions

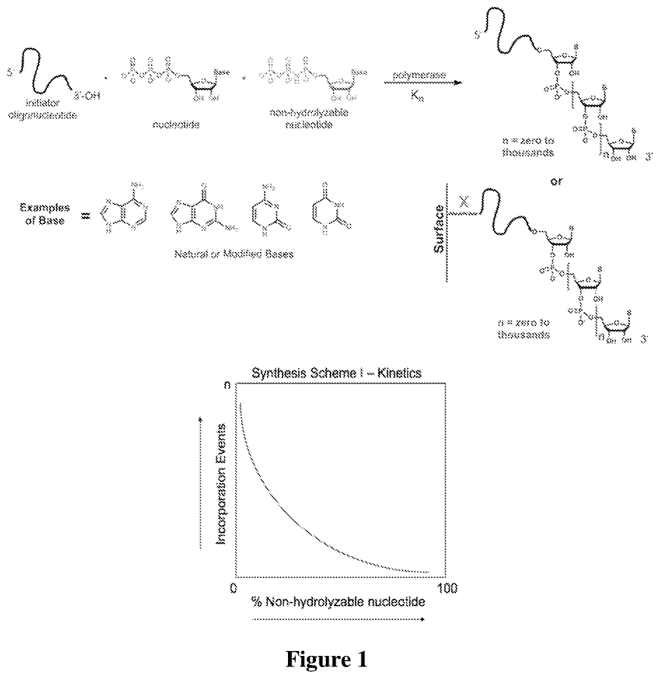

RNA oligonucleotide synthesis can be controlled by selecting reaction components that heavily impede natural nucleotide incorporation catalysis rates and maximize desired length products such as the addition of non-hydrolyzable or incompatible nucleotides (FIG. 1). Natural rNTPs are added to the 3′-terminus of an initiating RNA or DNA oligonucleotide hetero- or homopolymeric sequence of predetermined composition and length. The initiator oligonucleotide can be less than 20-nt. The initiating oligonucleotide may also include chemical modifications such as those that are photolabile or possessing electrochemical properties that allow for cleavage and separation from the full-length RNA oligonucleotide product after enzymatic synthesis. The number of incorporation events is proportional to the concentration ratio of the natural nucleotides to the non-hydrolyzable or incompatible nucleotides also present in the reaction vessel. The rate of oligonucleotide synthesis is directly impacted by this ratio through competitive inhibition, where low concentrations of non-hydrolyzable or incompatible nucleotides yield longer RNA oligonucleotides at higher reaction velocities and high concentrations of non-hydrolyzable or incompatible nucleotides yield small RNA oligonucleotides at slower reaction velocities. Non-hydrolyzable nucleotides include those with modifications to the α-, β-, or γ-phosphate of the triphosphate that do not affect the binding affinity of the nucleotide to the enzyme. Incompatible nucleotides include those with modifications to the 2′- and/or 3′-moieties, or nucleotide base that result in non-reactivity without significantly altering the binding affinity of the nucleotide to the enzyme. In one embodiment, this synthesis scheme can be carried out in a microfluidic setup in which different natural nucleotides may be switched out quickly so that multiple bases can be incorporated to a surface bound initiator oligonucleotide.

Modified rNTP Incorporation Reversibility Prevents Additional Incorporation Events

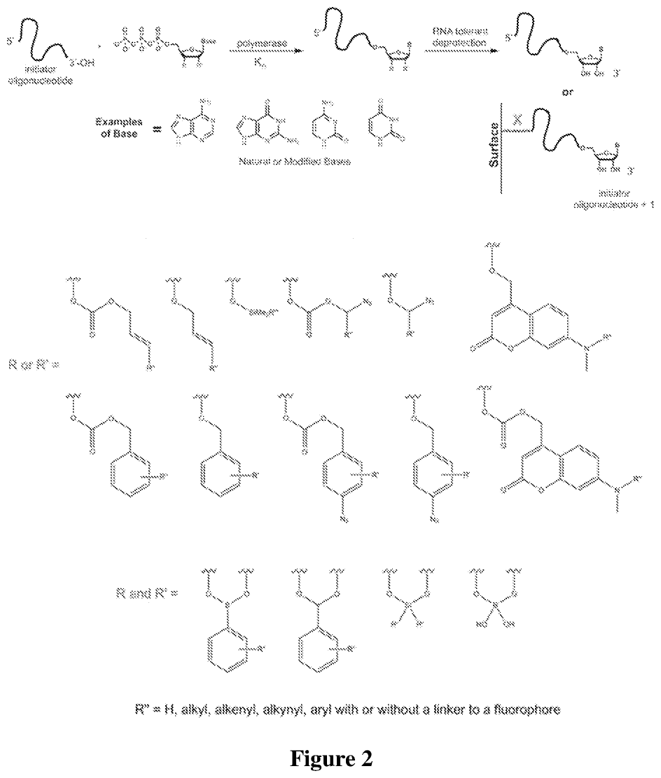

RNA oligonucleotide synthesis can be also controlled by incorporating modified nucleotides that temporarily alter the binding affinity of the polymerase to the initiator oligonucleotide in order to limit the extension reaction to just one incorporation event (n+1) (FIG. 2). Modified rNTPs are added to the 3′-terminus of an initiating oligonucleotide sequence of predetermined composition and length. The initiator oligonucleotide can be less than 20-nt. The initiating oligonucleotide may also include chemical modifications such as those that are photolabile or possessing electrochemical properties that allow for cleavage and separation from the full-length RNA oligonucleotide product after enzymatic synthesis. The incorporation of a single modified nucleotide alters the binding affinity of the enzyme towards the initiating oligonucleotide so that the enzyme can no longer incorporate additional nucleotides beyond the (n+1). The modified nucleotide can have, for example, non-natural chemical domains at the 2′-, 3′- or 2′- & 3′-positions of the nucleotide (FIG. 2). Once the modified rTNP is incorporated, a mild deprotection reaction is employed, which functions optimally under biologically compatible conditions, to remove the modification in order to reveal the natural chemical domain. Upon deprotection, the enzyme will have restored affinity towards the oligonucleotide and can incorporate an additional modified nucleotide corresponding to the next base of the sequence ((n+1)+1). The process of modified nucleotide incorporation, deprotection, and restoration of enzyme binding affinity is performed iteratively until the desired RNA oligonucleotide sequence is produced. A requirement of this scheme is that the conversion rate from the n to n+1 is very efficient, therefore steps will be taken to ensure that the enzyme has this capacity. In one embodiment, successful incorporation events may be monitored optically by selecting a modified nucleotide that includes a fluorophore or reactive domain for attaching a fluorophore post-incorporation. In another embodiment, this synthesis scheme can be carried out in a microfluidic setup that can be used to wash away unused nucleotide and prepare the extended RNA oligonucleotide for the next round of extension.

polymerase Enzymes for Controlled RNA Synthesis

### Family X Polymerases

polymerases from the Family X such as Terminal deoxynucleotidyl Transferase (TdT), polymerase Mu (Pol μ), polymerase Beta (Pol β), and polymerase Lambda (Pol λ) are candidates for the controlled, template-independent synthesis of RNA oligonucleotides (Fowler and Suo 2006). These highly specialized polymerases have been shown to be key driving forces in critical DNA repair pathways such as non-homologous end joining (NHEJ) and the generation of generation of antibody and T-cell receptor diversity during V(D)J recombination (Moon et al. 2007, 2014; Nick McElhinny and Ramsden 2004; Bertocci et al. 2006). The involvement of Family X polymerases in such biological processes are attributed to their precision in incorporating natural nucleotides in a template-dependent manner while maintaining the ability to indiscriminately add nucleotides to a primer sequence independently of a template when variability is necessary (J. F. Ruiz et al. 2001; Dominguez et al. 2000; Motea and Berdis 2010). This unique capacity is ideal for the enzymatic synthesis of RNA in that there is a large margin of natural flexibility associated with the Family X polymerases without the need for protein evolution schemes. It has been previously shown that TdT has the ability to incorporate natural nucleotides in addition to DNA nucleotides (Roychoudhury 1972). Family X polymerases could be further engineered to be more compatible with the proposed RNA synthesis schemes.

polymerase Mu (Pol μ)

Pol μ is a Family X polymerase that, under optimal reaction conditions, has been shown to efficiently incorporate both deoxyribonucleotide triphosphates (dNTPS) and rNTPs to DNA, RNA, and DNA-RNA hybrid oligonucleotide substrates (José F. Ruiz et al. 2003; Agrawal et al. 2003). Several studies of the enzyme's primary structure, catalytic pocket, and various catalytic states have determined the amino acid residues associated with rNTP binding and the kinetics of their incorporation (Moon et al. 2014; Jamsen et al. 2017; Moon et al. 2017). Interestingly, wild-type Pol μ has the ability to incorporate a rNTP without distorting the oligonucleotide primer or nucleotide structures as well as keeping the geometry of the active site in a normal configuration; a phenomena that may greatly affect the ability of other Family X polymerases to accommodate rNTPs in any useful capacity or speed (Moon et al. 2017). In addition, it is worth noting that Pol has been cited to be less discriminatory in its preference towards the DNA substrate compared to other Family X polymerases (Moon et al. 2015).

### Pol μ Mutagenesis

In one study, the expression of the two tumor associated human Pol μ point mutations, (G174S) and (R175H), produced enzymes with decreased efficiency and fidelity in NHEJ. These mutants were cited to randomly incorporate nucleotides despite having a template sequence guiding the DNA repair process resulting in significant alterations to the expected error-rates (Sastre-Moreno et al. 2017). Other groups have demonstrated that removing large proportions of Pol μ such as the N-terminal BRCT domain (which is typically associated with other DNA repair pathway core factors) result in active enzymes (Moon et al. 2014). These truncated variants were shown to retain wild-type activity and ability to bind non-hydrolyzable nucleotides, but potentially have more physical space to incorporate modified or bulky nucleotides. Nevertheless, wild-type Pol μ is characterized as a primarily template-dependent polymerase; however, the point mutation (R387K) to human wild-type Pol μ resulted in an enzyme with significant increased template-independent activity (Andrade et al. 2009). This point mutation (R387K) is exceedingly important to the feasibility using Pol in template-independent de novo RNA oligonucleotide synthesis and, again, reiterates the immense value of its flexibility. Pol μ is currently the only known polymerase out of the Family X and beyond that displays both template-dependent and template-independent activities (Domínguez et al. 2000; Juarez et al. 2006). In addition to a wild-type or mutagenized Pol μ, there are other polymerases that could be potentially used to synthesize long RNA oligonucleotides de novo.

### Poly(A) Polymerase, Poly(U) Polymerase, & Other RNA Polymerases

The 3′-tailing of single-stranded RNA with ribonucleotides is important in two different contexts: (1) natural biological or biochemical processes and (2) studying these processes in vivo or in vitro (Proudfoot 2011; Strauss et al. 2012). For the latter, several groups have employed wild-type RNA polymerases such as Saccharomyces cerevisiae and Escherichia coli poly(A) polymerase (PAP), Schizosaccharomyces pombe Cid 1 poly(U) polymerase (PUP) to directly label the 3′-terminus of RNA oligonucleotides in vitro in a template-independent manner (G. Martin and Keller 1998; Munoz-Tello, Gabus, and Thore 2012; Kwak and Wickens 2007; Winz et al. 2012). Under optimal conditions, it was shown that these families of enzymes, in particular, PAP, can accept modified ribonucleotides with modifications at the 2′- and 3′-positions of the sugar as well as the 8′-position of the adenosine base (Winz et al. 2012). While the overall incorporation efficiency varied between the modified position, nucleotide base, and the enzymes tested, 1-3 nucleotide incorporation events took place on average yielding a RNA oligonucleotide with a 3′- or internal azide functional group to be attached with a dye via bioorthogonal click chemistry (Winz et al. 2012).

In addition to studying the mechanism of modified nucleotide incorporation, other groups have examined both the biochemical and structural mechanisms for substrate binding and catalysis of PAP (Georges Martin et al. 2004; Bard et al. 2000). Through these studies, which included site-directed mutagenesis of many residues in the catalytic pocket and exhaustive analysis of steady-state kinetics, it was clear that there is that incorporation was heavily biased towards ATP over the other nucleotide bases (Georges Martin et al. 2004). However, another group determined that a single point mutation to a bacterial PAP (R215A) resulted in a complete reversal of this bias, allowing for random incorporation of all of nucleotide bases (Just et al. 2008). This result was similar for another template-independent RNA polymerase, CCA-adding enzyme (Just et al. 2008; Xiong and Steitz 2004). These studies make RNA polymerases such as PAP to be exceedingly compatible in the execution of scheme II for controlled de novo RNA oligonucleotide. Both the wild-type and mutagenized RNA polymerases appear to be flexible enough to incorporate U, A, G, or C sugar-modified nucleotides bases (2′-,3′-, or both) that can be deprotected or altered under mild reaction conditions to restore enzyme binding affinity.

### Results: Synthesis of RNA Oligonucleotides

### 1. Purified Human Polymerase μ R387K Displays Template-Independent Terminal Transferase Activity

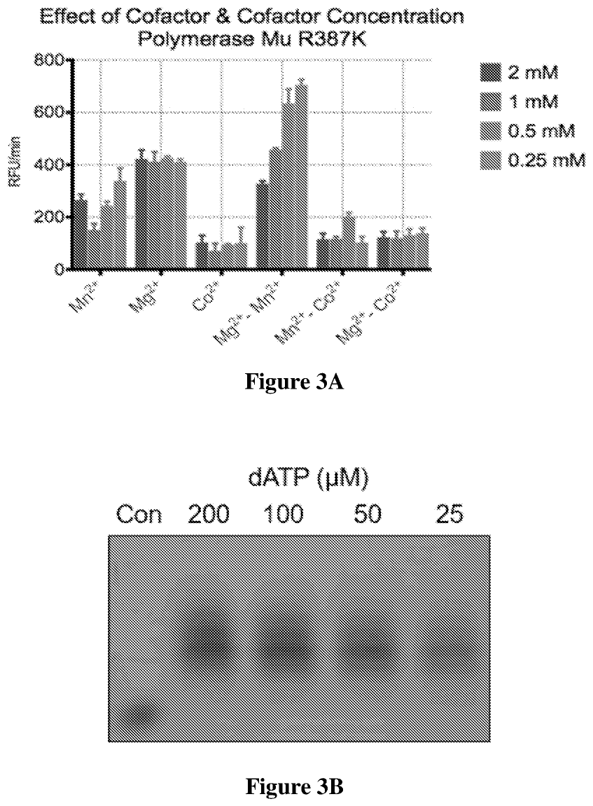

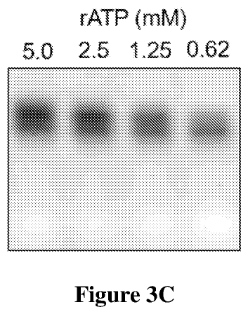

Human polymerase μ R387K was expressed and purified as described in the materials and methods. Because it was unknown under which reaction conditions polymerase μ R387K functioned optimally, several reaction parameters were initially evaluated. It was found that an incubation temperature 37° C. and the reaction buffer conditions: 10 mM Magnesium Acetate, 50 mM Potassium Acetate, and 20 mM Tris-Acetate yielded sufficient enzymatic activity in terms of dNTP incorporation. In addition, polymerase p activity was evaluated after reactions were supplemented with common divalent metal cofactors (Mn²⁺, Mg²⁺, Co²⁺, etc.) and it was found that a combination of Mn²⁺ and Mg²⁺ at a concentration of 0.25 mM yielded the highest rate of ssDNA generation at ˜650 RFU/minute, whereas 0.25 mM Co²⁺ yielded the worst rate at ˜100 RFU/minute (FIG. 3A). Next, it was sought to determine if polymerase μ R387K can incorporate rNTPs under similar reaction conditions. Denaturing gel electrophoresis indicated that polymerase μ R387K can incorporate both incorporate natural dATP (FIG. 3B) and rATPs (FIG. 3C); however, a concentration of 5 mM of rATP was needed to produce a response similar to 200 μM of dATP. These results indicate that polymerase μR387K displays terminal-transferase activity like other polymerase X Family members, functions independently of a template sequence, and can accommodate both dNTPs and rNTPs. Thus, polymerase p R387K, a confirmed DNA/RNA-directed polymerase, is useful for controlled enzymatic RNA synthesis.

2. S. cerevisiae Poly(A) Polymerase Incorporates 2′-Modified ATP Nucleotides and 2′-Blocked Reversible Terminators

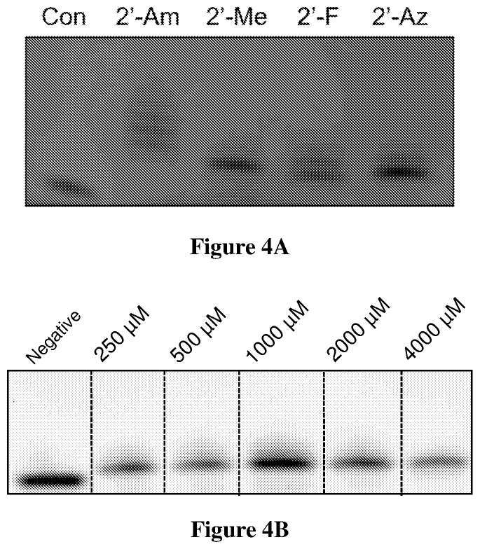

The efficiency of S. cerevisiae poly(A) polymerase (Thermo 74225Z25KU) for incorporating of several 2′-modified was evaluated. The modified nucleotides evaluated included the following: 2′-F-rATP (Trilink N-1007), 2′-Azido-rATP (Trilink N-1045), 2′-Amino-rATP (Trilink N-1046), and 2′-O-Methyl rATP (Trilink N-1015). Extension reactions were supplemented with the appropriate buffers, which included 0.5 mM Mn²+, 200 pmol of the initiator RNA oligonucleotide, 2.5 mM modified nucleotide, and 900 units of enzyme. Reactions were incubated at 37° C. for 60 minutes before being analyzed with denaturing gel electrophoresis. According to the gel (FIG. 4A), under these reactions conditions S. cerevisiae poly(A) polymerase incorporates all 2′-modified nucleotides (2′-Amino-, 2′-O-Methyl-, 2′-F-, and 2′-Azido-rATP). These results indicate that S. cerevisiae poly(A) polymerase is tolerant to different chemical modifications on the 2′-moiety of nucleotide sugar and would be compatible with the 2′-reversible terminator chemistry. Under the same reaction conditions, the reversible terminator nucleoside triphosphate, 2′-O-allyl-rATP, was tested for controlled incorporation by S. cerevisiae poly(A) polymerase. The resultant denaturing gel (FIG. 4B) indicated positive (n+1) incorporation over a range of nucleoside triphosphate concentrations (250 μM to 4000 μM) as compared to a negative control reaction that contained all components besides enzyme and nucleotide. This indicates that the combination of S. cerevisiae poly(A) polymerase and reversible terminator nucleoside triphosphates bearing the 2′-O-allyl can be used for the controlled enzymatic synthesis of RNA oligonucleotides.

3. S. pombe Cid1 poly(U) polymerase Incorporates Natural Nucleotides Universally

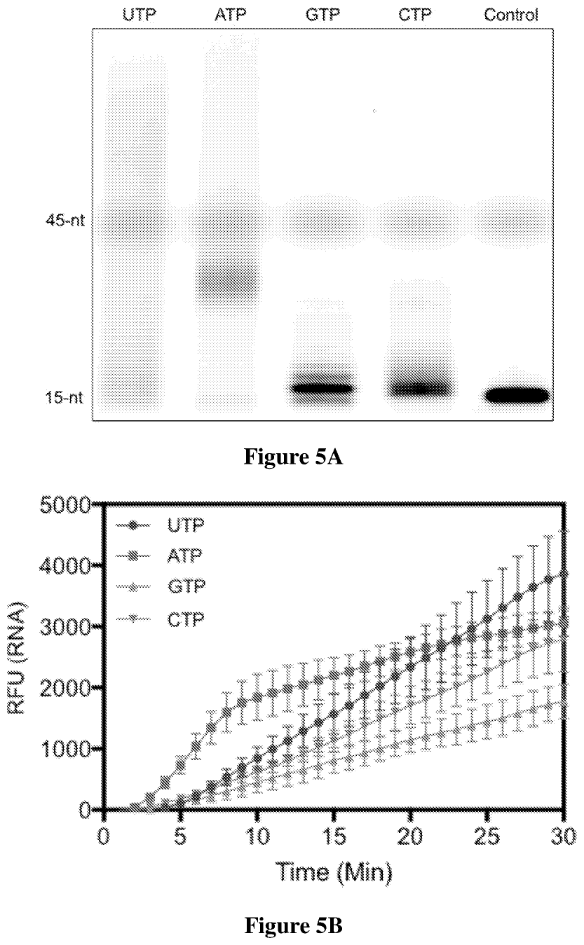

The efficiency of S. pombe Cid1 poly(U) polymerase (NEB M0337) for incorporating natural ribonucleotides evaluated. For kinetic analysis, extension reactions were supplemented with the appropriate buffers, 10 pmol of the labeled initiator RNA oligonucleotide (5′-Cy5-poly rU-15-mer), 1.0 mM natural nucleotide (either ATP, UTP, GTP, or CTP), 1×SYBR Green II for RNA (Thermo), and 2 units of poly(U) polymerase. Reactions were incubated at 37° C. for 30 minutes and monitored in real time. 2 μL of each extension reaction was then analyzed using a 15% TBE-Urea gel and imaged on a Typhoon FLA 9500 system with EX: 649 nm and EM: 666 nm. It is clear from the gel that poly(U) polymerase has the ability to incorporate all-natural ribonucleotides as compared to the control; however, there is some bias in terms of how many extensions will occur (FIG. 5A). These results are consistent with previous work (Munoz-Tello, Gabus, and Thore 2012; Lunde, Magler, and Meinhart 2012), with the exception of rGTP and rCTP in that poly(U) polymerase has been previously indicated to display little to no activity with these nucleotides. However, it was confirmed that poly(U) polymerase has activity with rGTP and rCTP using the results from the gel electrophoresis and RNA kinetic assay (FIG. 5B).

4. S. pombe Cid1 poly(U) polymerase Incorporates 2′-Modified Nucleotides Universally

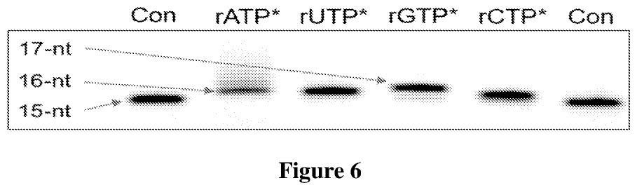

Knowing that S. pombe Cid1 poly(U) polymerase has the ability to incorporate all four natural ribonucleotides universally, it was sought to determine if this universality can be extended to 2′-modified nucleotides. Using the same reaction parameters, poly(U) polymerase was incubated with 2.5 mM 2′-O-Methyl-rATP, rUTP, rCTP or rGTP at 37° C. for 60 minutes. 2 μL of each extension reaction was then analyzed using a 15% TBE-Urea gel and imaged on a Typhoon FLA 9500 system with EX: 649 nm and EM: 666 nm. The gel indicates S. pombe Cid1 poly(U) polymerase incorporates the 2′-modified nucleotides universally and only extends the initiator oligonucleotide by +1-2 nucleotides with very high efficiently (FIG. 6). As before, there is some bias in nucleotide selection; however, this is overall negligible as all extension products are very similar after the 60-minute incubation. This result is exceedingly ideal for controlled RNA synthesis and S. pombe Cid1 poly(U) polymerase is unique in that (1) no other currently known enzyme can incorporate modified nucleotides universally and (2) the extension product is only+1-2 nucleotides. Like S. cerevisiae poly(A) polymerase, S. pombe Cid1 poly(U) polymerase is tolerant to chemical modifications on the 2′-moiety of nucleotide sugar and would be compatible with the 2′-reversible terminator chemistry.

5. S. pombe Poly(U) Polymerase is Minimally Affected by Initiator Oligonucleotide Sequence Composition and Secondary Structure/Hairpins

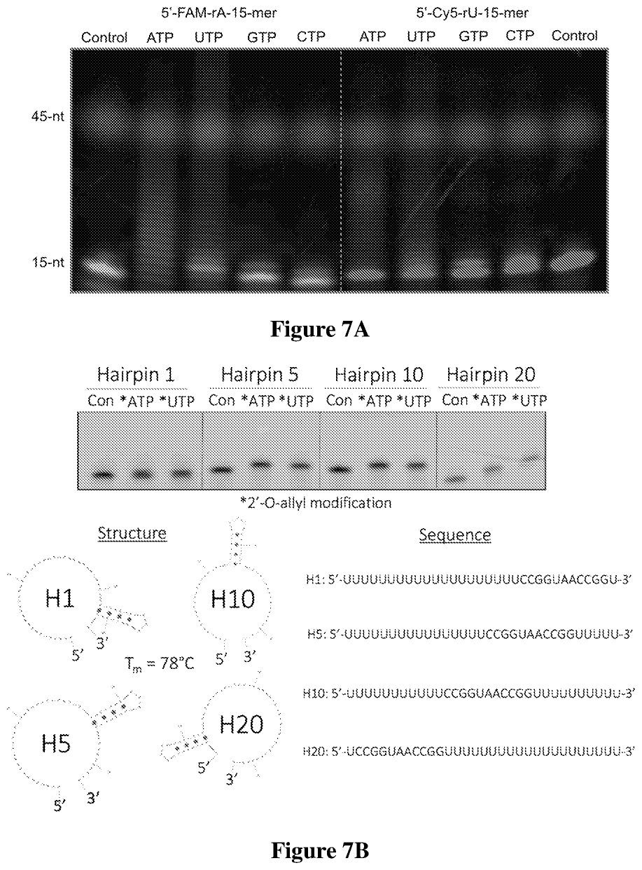

Many terminal transferases are extremely sensitive to the sequence composition of the initiator oligonucleotide, where a different base at the 3′-OH terminus can greatly affect the rate at which a nucleotide is incorporated. Thus, it was investigated whether S. pombe poly(U)polymerase is affected in this manner by performing extension reactions for all four natural ribonucleotides in the presence of two 5′-labeled initiator oligonucleotides with differing compositions. Reactions were performed with 1 mM ribonucleotide and 10 pmol of the 5′-labeled initiator oligonucleotide. 2 μL of each extension reaction was then analyzed using a 15% TBE-Urea gel and imaged on a Typhoon FLA 9500 system with EX: 649 nm and EM: 666 nm for the 5′-Cy5-poly rA-15-mer and EX: 495 nm and EM: 520 nm. It was found that S. pombe poly(U) polymerase is minimally affected by initiator oligonucleotide sequence composition bias as indicated by denaturing gel electrophoresis (FIG. 7A). It was observed that both initiator oligonucleotides yielded universal ribonucleotide incorporation; however, it appeared that the 5′-Cy5-poly rA-15-mer was slightly more efficient as less starting product was present after the 30-minute incubation. In addition to sequence composition, the effect of controlled enzymatic extension using initiator oligonucleotides with strong secondary structure was investigated. In order to do this, several oligonucleotides were generated using the IDT Oligoanalyzer Tool™ to induce strong hairpin structures under reaction conditions (taking into consideration Mg²⁺, nucleotide concentration, DNA/RNA oligonucleotide, etc). Each oligonucleotide was similar in sequence except that the location of the hairpin in comparison to the 3′-terminus was varied to produce the following: 1 base from the 3′-terminus (H1), 5 bases from the 3′-terminus (H5), 10 bases from the 3′-terminus (H10), and 20 bases from the 3′-terminus. In order to ensure that oligonucleotides' hairpins were correctly formed before enzymatic extension, the oligonucleotides were heated to 95° C. and then slowly cooled at a rate of 0.1° C./min to 15° C. in the appropriate enzymatic reaction buffer on a thermocycler. After cooling, the remaining reaction components were added to the hairpin initiator oligonucleotide and the extension reaction was carried out for 5 minutes at 37° C. using either the reversible terminator nucleoside triphosphate 2′-O-allyl-ATP or -UTP. A 15% TBE-urea gel under denaturing conditions was used to determine the efficiency of extension for each of the hairpin initiator oligonucleotides. Overall, S. pombe poly(U) polymerase was able to extend hairpin initiator oligonucleotides with strong secondary structure to a (n+1); however, it did have some difficulty with the H1 oligonucleotide where there was only one 1 base following the hairpin (FIG. 7B)—about 10% extension yield. Difficult secondary structure may potentially be problematic for the synthesis scheme, but additional reaction components such as DMSO or Betaine could be added to help in addition to high reaction temperatures or poly(U) polymerase mutants. Nevertheless, these results further underline the unique flexibility of S. pombe poly(U) polymerase.

6. S. pombe Poly(U) Polymerase Activity is Enhanced by the Addition of Inorganic Pyrophosphatase

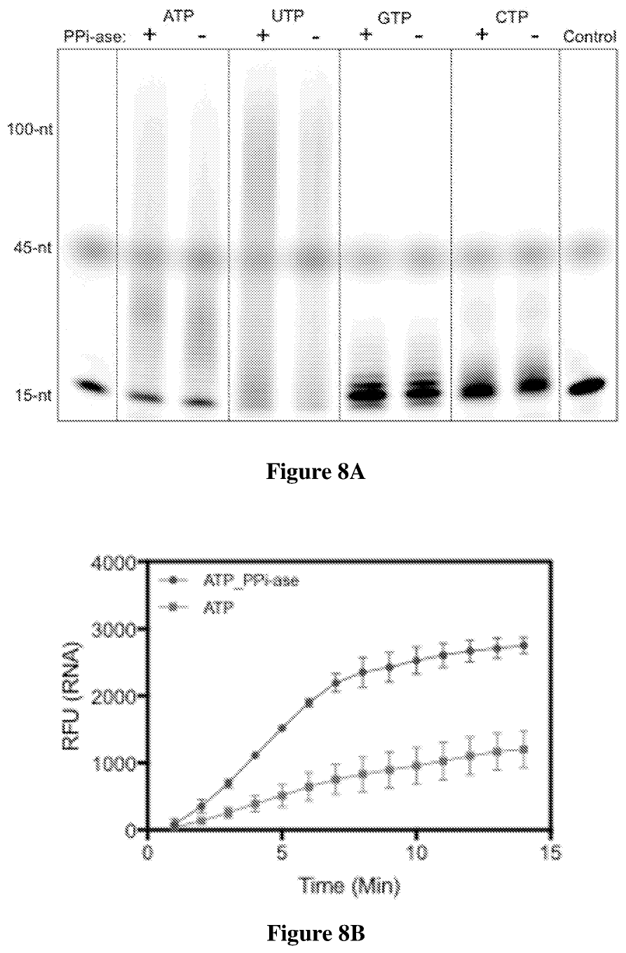

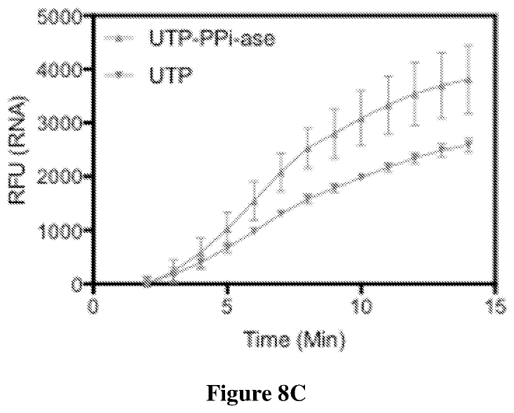

A consequence of high terminal transferase activity is the fast accumulation of inorganic pyrophosphate, a known inhibitor of DNA- and RNA-directed polymerases. In order to reduce the accumulation of inorganic pyrophosphate, an inorganic pyrophosphatase (PPi-ase) can be employed to cleave the pyrophosphate into two single phosphates while the reaction progresses. Therefore, it was sought to determine if the supplementation of pyrophosphatase can enhance the terminal transferase activity of S. pombe poly(U) polymerase. Reactions were incubated for 30 minutes at 37° C. with 1 mM of each ribonucleotide, 10 pmol of 5′-Cy5-poly-rU-15-mer initiator oligonucleotide, and 0.1 units of Yeast Inorganic Pyrophosphatase (New England Biolabs M2403). 2 μL of each extension reaction was then analyzed using a 15% TBE-Urea gel and imaged on a Typhoon FLA 9500 system with EX: 649 nm and EM: 666 nm. It was found that the supplementation of inorganic pyrophosphatase enhances the rate at which S. pombe poly(U) polymerase synthesizes RNA and increases the maximum length of the synthesized RNA (FIGS. 8A to 8C). The greatest enhancement was observed for the natural ribonucleotides rUTP and rATP, while rGTP and rCTP were minimally affected. This observation is primarily due to the preference of the wild-type S. pombe poly(U) polymerase for rUTP and rATP in synthesizing long RNA strands and thus, more accumulation of inorganic pyrophosphate. While rGTP and rCTP are still able to be incorporated by S. pombe poly(U) polymerase, the total number of incorporation events is fewer which results in less accumulation of inorganic pyrophosphate. Nevertheless, the addition of inorganic pyrophosphatase to S. pombe poly(U) polymerase RNA synthesis reactions is beneficial and comprises a modification to the recommended and commercially standardized reaction conditions.

7. S. pombe Poly(U) Polymerase Activity can Naturally Incorporate Base-Modified Ribonucleotides

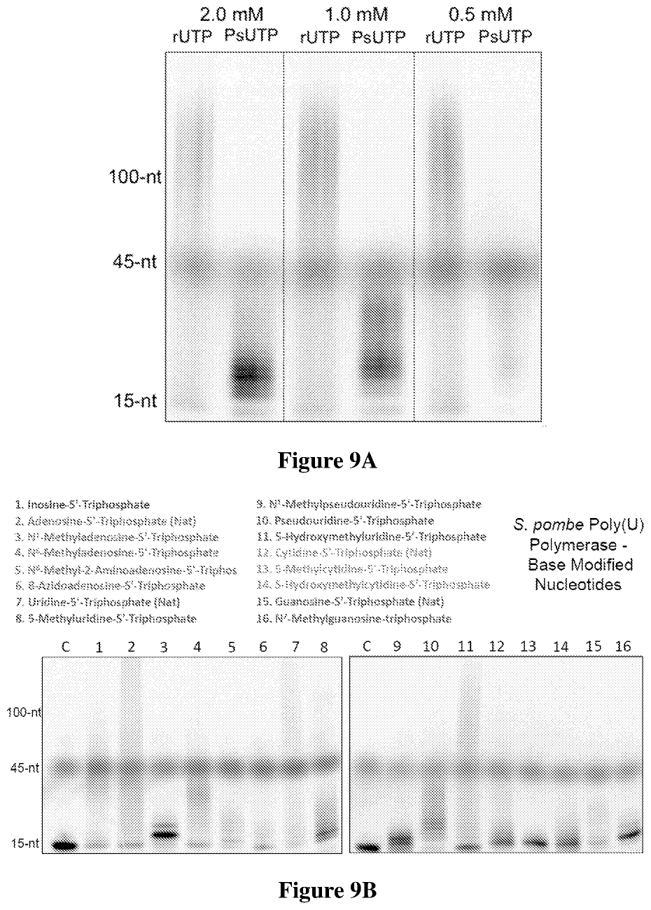

An application of the methods provided herein is the synthesis of biologically active molecules such as synthetic transfer RNA (tRNA) or ribosomal RNA (rRNA). Often the ribonucleotide bases that comprise tRNA and rRNA are naturally base modified to, for example, induce secondary structure in vivo for optimal functionality. Additionally, the incorporation of modified bases into RNA oligonucleotides can greatly enhance their stability and protect against unwanted nuclease digestion. Thus, it was sought to determine if S. pombe poly(U) polymerase has the ability to incorporate Pseudouridine, one of the most commonly found modified ribonucleotide bases in tRNA and rRNA. Reactions were incubated for 30 minutes at 37° C. with 2 mM, 1 mM, or 0.5 mM rUTP or Pseudouridine (Trilink N-1019), 10 pmol of 5′-Cy5-poly-rU-15-mer initiator oligonucleotide, and 0.1 units of Yeast Inorganic Pyrophosphatase. 2 μL of each extension reaction was then analyzed using a 15% TBE-Urea gel and imaged on a Typhoon FLA 9500 system with EX: 649 nm and EM: 666 nm. It was found that S. pombe poly(U) polymerase has the innate ability to incorporate Pseudouridine, producing based-modified RNA oligonucleotides approximately 30-45-nt in length (FIG. 9A). The resultant poly-Pseudouridine RNA oligonucleotides were shorter than the un-modified rUTP oligonucleotide; however, this is the first known instance demonstrating that S. pombe poly(U) polymerase can incorporate such nucleotides. It is clear from these results, that S. pombe poly(U) polymerase will most likely have the ability to incorporate other base modified nucleotides such as those with methylations or other modifications at various positions on the nucleotide base. Thus, S. pombe poly(U) polymerase was incubated with an array of base modified nucleoside triphosphates with modifications to all four natural bases. These included, but are not limited to, inosine 5′-triphosphate, N¹-methyladenosine-5′-triphosphate, N⁶-methyladenosine-5′-triphosphate, N⁶-methyl-2-aminoadenosine-5′-triphosphate, 8′-azidooadenisone-5′-triphosphate, 5-methyluridine-5′-triphosphate, N¹-methylpseudouridine-5′-triphosphate, pseudouridine-5′-triphosphate, 5-hydroxymethyluridine-5′-triphosphate, 5-methylcytidine-5′-triphosphate, 5-hydroxymethylcytidine-5′-triphosphate, N⁷-methylguanosine-triphosphate, intrinsically fluorescent nucleotides such as 3′/2′-O—(N-Methyl-anthraniloyl)-triphosphate and phosphate modified nucleotides such as α-, β-, γ-thio triphosphates. Denaturing gel electrophoresis revealed incorporation of each base modified ribonucleotide tested with varying numbers of incorporation events (FIG. 9B). Base modified ribonucleotides like those tested can be synthesized with the 2′-blocking group to make them compatible as a reversible terminator. These results form the basis for the de novo synthesis of important biological molecules such as tRNA or rRNA that can be heavily base modified, depending on their function or RNA oligonucleotides with specific secondary structure or RNA oligonucleotide with increased resistance to degradation.

8. RNA Synthesis by S. pombe Poly(U) Polymerase can be Controlled with Competitive Inhibitor Nucleotides

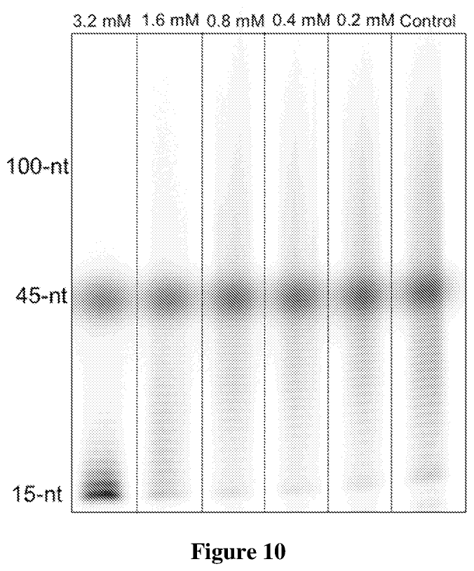

As exemplified in FIG. 1, the rate of RNA oligonucleotide synthesis is directly impacted by the ratio of hydrolyzable nucleotides and non-hydrolyzable nucleotides present in the reaction via competitive inhibition. Inhibitors occupy the enzyme's active site but are not incorporated, which slows down the RNA synthesis reaction and subsequently, the number of incorporation events within a given reaction time. To demonstrate this, S. pombe poly(U) polymerase RNA synthesis reactions were incubated with increasing concentrations of the non-hydrolyzable ribonucleotide uridine-5′-[(α,β)-imido]triphosphate (Jena Biosciences). It was found that higher concentrations of the non-hydrolyzable ribonucleotide significantly limited the total number of hydrolyzable rUTP incorporations (FIG. 10). RNA synthesis reactions were incubated for 30 minutes at 37° C. and 2 μL of each extension reaction was then analyzed using a 15% TBE-Urea gel and imaged on a Typhoon FLA 9500 system with EX: 649 nm and EM: 666 nm. Concentrations of the non-hydrolyzable ribonucleotide Uridine-5′-[(α,β)-imido]triphosphate below 0.8 mM had little effect on controlling the RNA synthesis reaction; which is likely due to the binding affinity threshold of the Uridine-5′-[(α,β)-imido]triphosphate to S. pombe poly(U) polymerase. Other non-hydrolyzable ribonucleotides can be employed in this manner, and they need not be uridine bases nor the polymerase S. pombe poly(U) polymerase.

9. RNA Synthesis by S. pombe Poly(U) Polymerase can be Controlled with 2′-O-Blocked Reversible Terminator Nucleoside Triphosphates

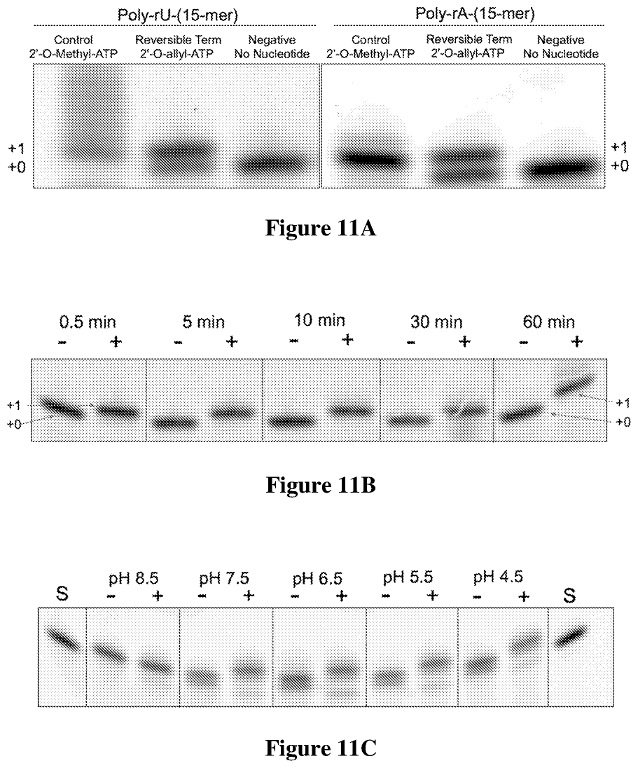

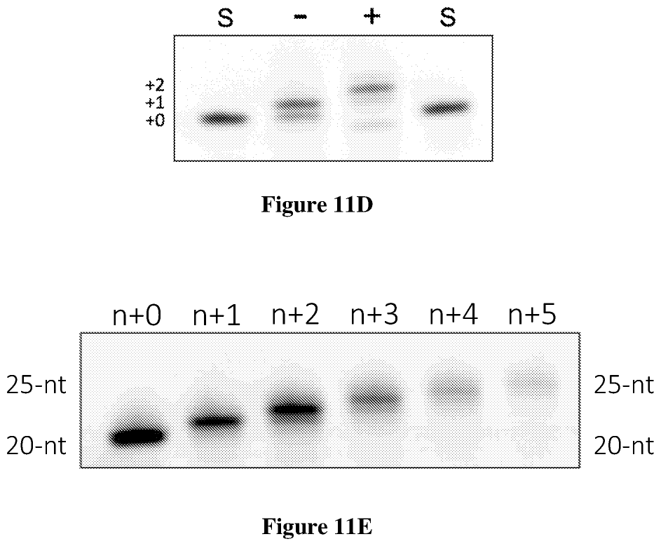

As exemplified by FIG. 2, RNA synthesis can be controlled by incorporating a 2′-O-blocked reversible terminator nucleoside triphosphate whereby synthesis is temporarily terminated after a single incorporation event resulting in a (n+1) oligonucleotide. A gentle deprotection scheme is then employed to remove the blocking group to re-establish the growing oligonucleotide as a recognizable substrate for the enzyme such as poly(U) polymerase. In order to determine if S. pombe poly(U) polymerase has the ability to incorporate reversible terminator 2′-modified nucleoside triphosphates, 2′-O-allyl-ATP was incubated in the RNA synthesis reaction for 30 minutes at best reaction conditions. The resulting analysis of the RNA synthesis reactions indicated that S. pombe poly(U) polymerase incorporated only a single 2′-O-allyl-ATP and extended the initiator oligonucleotide by one base as compared to control reactions (FIG. 11A). This result is of particular note as many other modified nucleoside triphosphates (such as 2′-, base, etc.) result in multiple extension products, while the 2′-O-allyl-ATP only results in one extension product. Further optimization of reactions conditions and component stoichiometry resulted in determining that S. pombe poly(U) polymerase requires less than or equal to 0.5 minutes to fully convert the initiator oligonucleotide (+0) to an extension product (+1) with >99% conversion rates (FIG. 11B). Further increase in incubation time did not have any appreciable effects on the RNA synthesis reactions. The deblocking and further extension of the reversible terminator 2′-modified nucleoside triphosphates 2′-O-allyl-ATP was demonstrated by incubating purified (n+1) oligonucleotide with a mixture of Disodium tetrachloropalladate (Na₂PdCl₄) and 3,3′,3″-Phosphanetriyltris(benzenesulfonic acid) trisodium salt (TPPTS) in a Tris-HCl buffer at variable pH's for 10 minutes at 50° C. The resulting deblocked oligonucleotide was then purified and then extended with the optimized S. pombe poly(U) polymerase reaction conditions which included 1 mM reversible terminator 2′-O-allyl-ATP in order to obtain the (n+2) product. Denaturing gel electrophoresis analysis of indicated that the purified (n+1) oligonucleotide was converted to a (n+2) product with high efficiency after an optimization of deblocking buffer composition and pH (FIG. 11C). Successful deblocking of the 2′-reversible terminator group was observed in Tris-HCl buffer with pH 7.5, 6.5, 5.5, 4.5, but not 8.5. Lower pH buffers are known to those familiar with the art to enhance RNA stability and the minimal buffer composition ensures that the RNA oligonucleotide is accessible by the deblocking components (Pd & TPPTS). A high-resolution denaturing gel showing the (n+2) product is shown in FIG. 11D. Additionally, the stability and functionality of the 5′-fluorophore was retained throughout the deblocking process and minimal oligonucleotide degradation was observed demonstrating that the chemistry is biocompatible. To determine if the 2′-O-allyl reversible terminator strategy was conducive to the synthesis of longer RNA oligonucleotide fragments, the process of enzymatic extension and deblocking was repeated in order to reach an (n+5) product using optimized reaction conditions and 2′-O-allyl-ATP in bulk solution. During synthesis, a small aliquot of each extension/deblocking event (also known as a synthesis cycle) was set aside for analysis. Gel electrophoresis analysis indicated a successful synthesis of a 25-nt fragment starting from a 20-nt initiator oligonucleotide (FIG. 11E). Note that sample loss did occur during each purification and deblocking step, which is indicated by a decreasing signal on the gel. Overall, these results indicate S. pombe poly(U) polymerase has the innate capacity to perform enzymatic RNA synthesis in an iterative, base-by-base manner as outlined in FIG. 2. Other reversible terminator nucleoside triphosphates may be also incorporated by S. pombe poly(U) polymerase. This includes, but is not limited, to 2′-O-azido-methyl-NTP.

10. S. pombe Poly(U) Polymerase Efficiently Incorporates 2′-Reversible Terminator Nucleoside Trisphosphates with All Four Natural Nucleobases

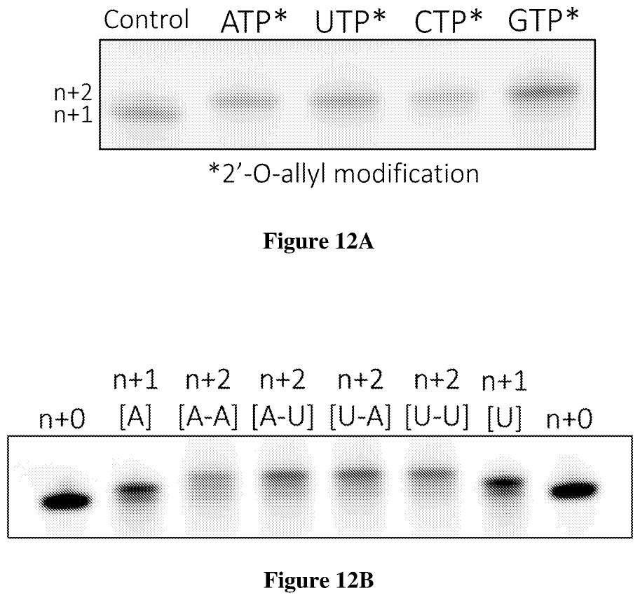

Previously, it was determined that S. pombe poly(U) polymerase has the ability to incorporate 2′-modified nucleoside triphosphates (2′-methoxy) bearing the four natural RNA nucleobases (A, U, G, C) with relatively equal efficiency (FIG. 6). In order to determine whether this was the case for reversible terminator nucleoside triphosphates, 2′-O-allyl-ATP, -UTP, -CTP, and -GTP were synthesized. Using optimized conditions, extension reactions were incubated for 1 minute at 37° C. in a thermocycler with either 1 mM of 2′-O-allyl-ATP, -UTP, -CTP, or -GTP reversible terminator. Reactions were then analyzed using a 15% TBE-urea denaturing gel revealing that each nucleobase version of the 2′-O-allyl reversible terminator is efficiently incorporated by S. pombe poly(U) polymerase as indicated by a single (n+1) extension event (FIG. 12A). The control reaction had all reaction components except nucleotide. In order to further prove that this result was conducive for multi-nucleobase RNA oligonucleotide synthesis, binary synthesis using combinations of 2′-O-allyl-ATP and -UTP was performed. The following combinations were tested: (n+1) A, (n+1) U, (n+2) A-A, (n+2) A-U, (n+2) U-A, and (n+2) U-U using optimized enzymatic extension and deblocking reaction conditions in bulk solution. The resultant material was analyzed using a 15% TBE-urea gel, which indicated positive incorporation for the single (n+1) test conditions and positive incorporation and deblocking for the double (n+2) test conditions as compared to a blank reaction (all components except nucleotide) that yielded the (n+0) example (FIG. 12B).

11. Active S. pombe Poly(U) Polymerase can be Expressed in Bacteria with an N-Terminus His⁶-Tag for Large-Scale Production and Purification

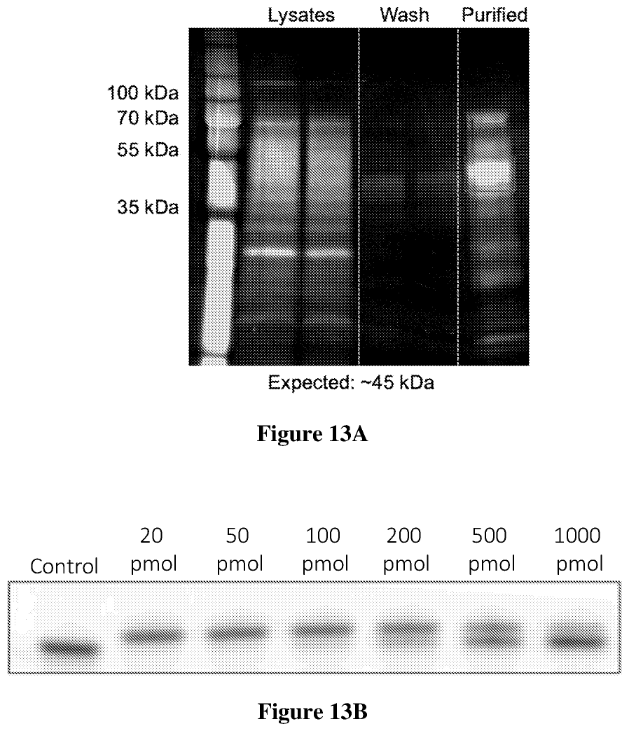

The primary sequence for S. pombe poly(U) polymerase (UniProtKB—O13833) was modified by adding the amino acids “MGSSHHHHHHSSGLVPRGSH” to the N-terminus of the enzyme. These amino acids encode for an N-terminus His⁶-tag with the appropriate linkers. Using the protocol outlined in the material and methods section, N-terminus His⁶-tagged S. pombe poly(U) polymerase was expressed, purified, and concentrated to a small volume. Denaturing gel electrophoresis indicated that N-terminus His⁶-tagged S. pombe poly(U) polymerase was properly expressed and isolated from bacterial lysates. With the N-terminus His⁶-tag, the expected molecular weight of S. pombe poly(U) polymerase is approximately 45 kDa—which a strong band corresponded to on the gel (FIG. 13A). In order to determine the activity of the purified and concentrated N-terminus His⁶-tagged S. pombe poly(U) polymerase, extension reactions supplemented with 1 mM 2′-O-allyl-ATP reversible terminator nucleotide and increasing amounts of initiator oligonucleotide were incubated with the expressed enzyme. Using the N-terminus His⁶-tagged S. pombe poly(U) polymerase, it was determined that approximately 100 pmol of initiator oligonucleotide can be converted to the (+1) product with a conversion rate of >99% (FIG. 13B). Reactions supplemented with greater than 100 pmol may require additional optimization for a higher conversion rate. These results indicate that the N-terminus His⁶-tagged S. pombe poly(U) polymerase can be readily expressed for very low costs and can be scaled up to yield appreciate quantities of the enzyme for RNA synthesis. Furthermore, the purified and concentrated N-terminus His⁶-tagged S. pombe poly(U) polymerase can convert large amounts of RNA oligonucleotide material, thereby reducing the need for repeated synthesis reactions to obtain large yields of the desired RNA sequence. No RNase carry-over from the bacterial lysates in which S. pombe poly(U) polymerase was expressed and purified from was observed.

12. Controlled RNA Oligonucleotide Synthesis Using S. pombe Poly(U) Polymerase can be Carried Out on Solid-Phase Surfaces

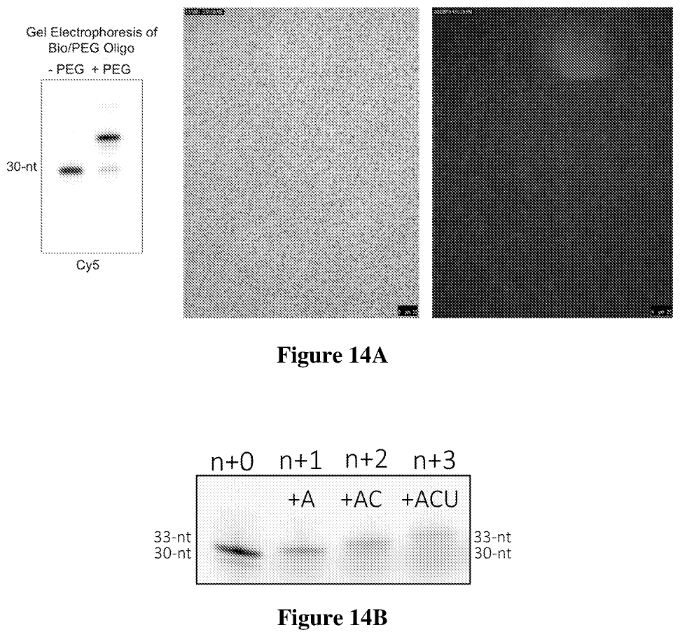

Controlled RNA oligonucleotide synthesis can be readily performed using bulk solutions; however, after the extension and deblocking steps in each synthesis cycle the growing oligonucleotide must be purified to remove interfering components. Multiple purifications, while having efficient recovery with modern methods, ultimately lead to major sample loss after several cycles of synthesis. Thus, performing oligonucleotide synthesis on a solid-phase support such as functionalized beads, wells, slides, etc. is significantly more conducive for the synthesis of longer oligonucleotide fragments and large, industrially relevant quantities of material. In order to evaluate the ability for S. pombe poly(U) polymerase to extend oligonucleotides anchored to a surface, an initiator oligonucleotide bearing a 5′-amine group and internal Cy5 dye was first used to attach a 5′-Biotin-PEG-NHS linker (EZ-Link #A35389 Thermo). The efficiency the labeling reaction was determined to be >90% via analysis with a 15% TBE-urea gel (the addition of the bulky PEG group will make the oligonucleotide run differently as compared to the non-labeled oligonucleotide) (FIG. 14A). Further quality control was performed using Streptavidin functionalized magnetic beads (Spherotech #SVM-20-10), where it was observed that positive non-covalent anchoring of the labeled initiator oligonucleotide with the internal Cy5 dye occurred as determined by fluorescence microscopy (FIG. 14A). Three cycles of enzymatic oligonucleotide synthesis were then conducted using optimized extension and deblocking reaction conditions using the anchored initiator oligonucleotide and 2′-O-allyl reversible terminator nucleoside triphosphates. An (n+3) product was synthesized where cycle 1 added an “A”, cycle 2 added an “C”, and then cycle 3 added an “U”. Post-synthesis, the anchored oligonucleotide was removed from the streptavidin beads' surface by incubating the beads with a solution of 90% formamide in water at 50° C. for 10 minutes in a thermocycler. The collected material was purified and analyzed using a 15% TBE-urea gel, which indicated highly efficient oligonucleotide synthesis with multiple nucleobases as catalyzed by S. pombe poly(U) polymerase (FIG. 14B). A major advantage to solid-phase synthesis over bulk synthesis is that extension and deblocking reactions can be repeated to ensure the either reaction is fully completed with purification. This could be visualized or measured by monitoring the generation of pyrophosphate during extension reactions, measuring the solid-support fluorescence if a dye-labeled nucleoside triphosphate is used, or using colorimetric monitor of the deblocking reaction.

13. A Reusable Solid-Phase Support with Covalent Linker can be Used for S. pombe Poly(U) Polymerase Mediated Controlled Enzymatic RNA Oligonucleotide Synthesis

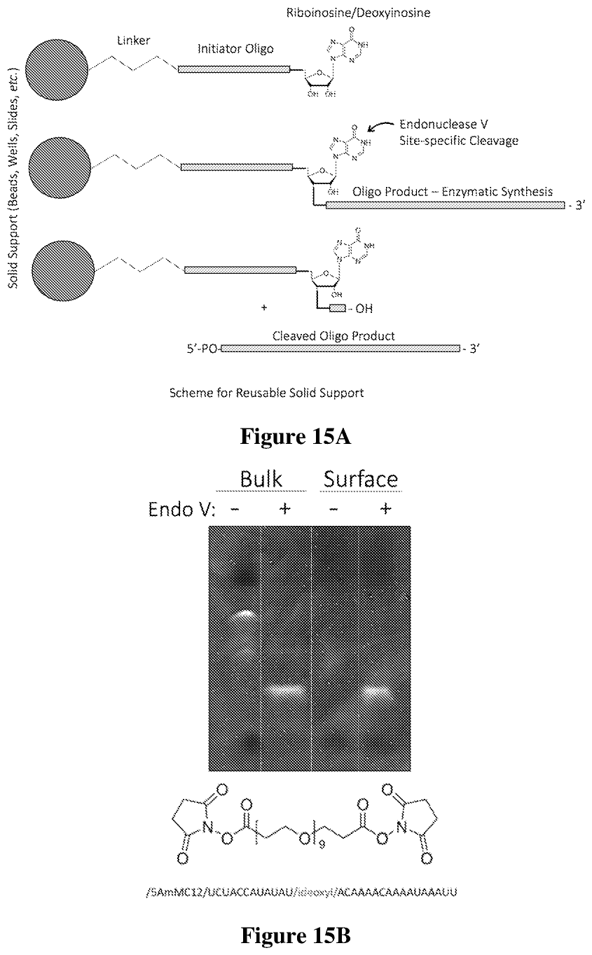

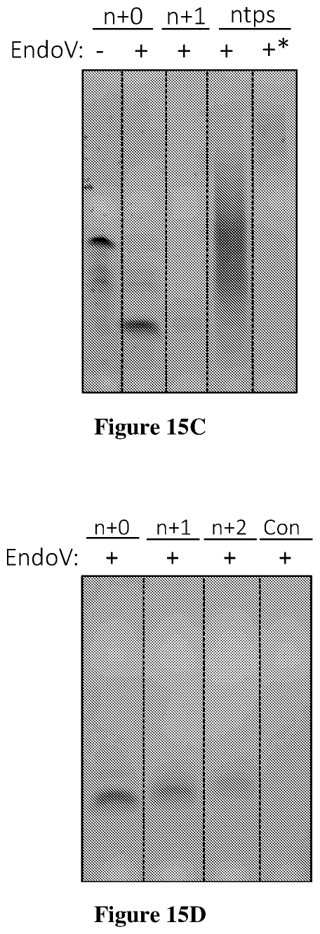

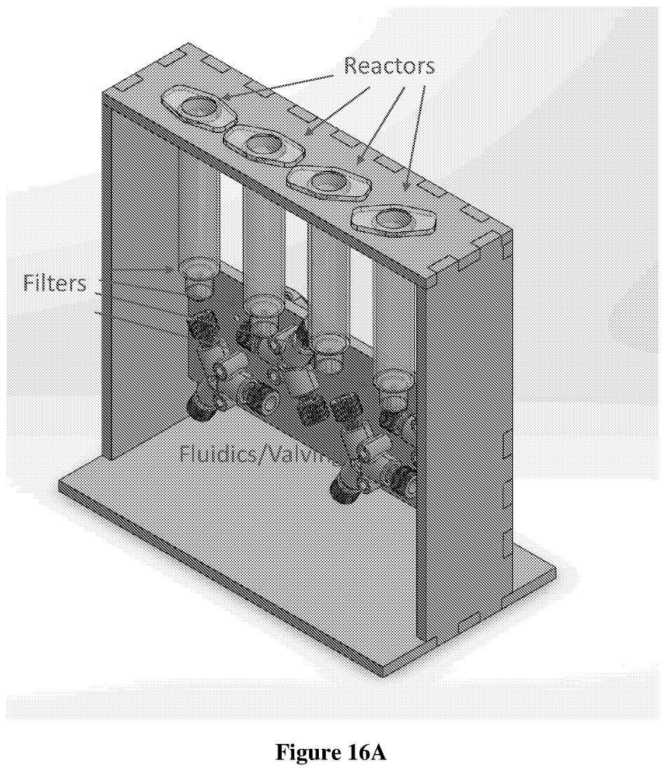

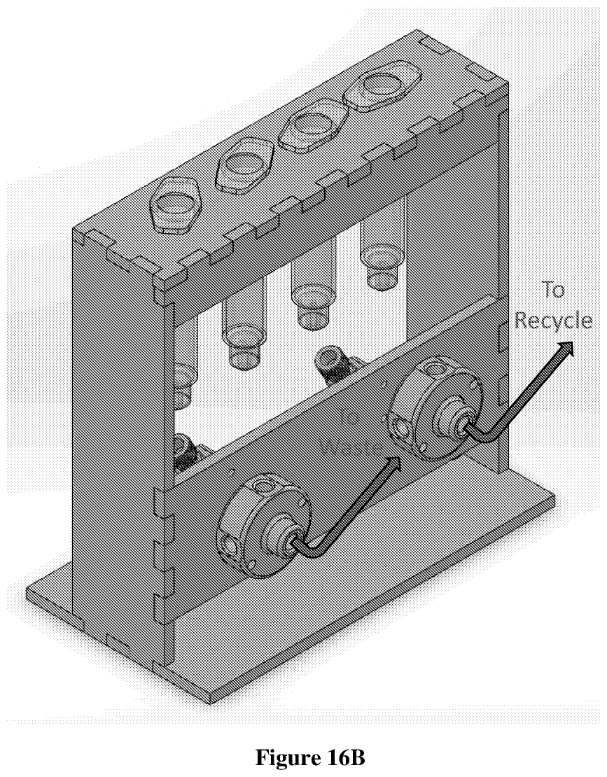

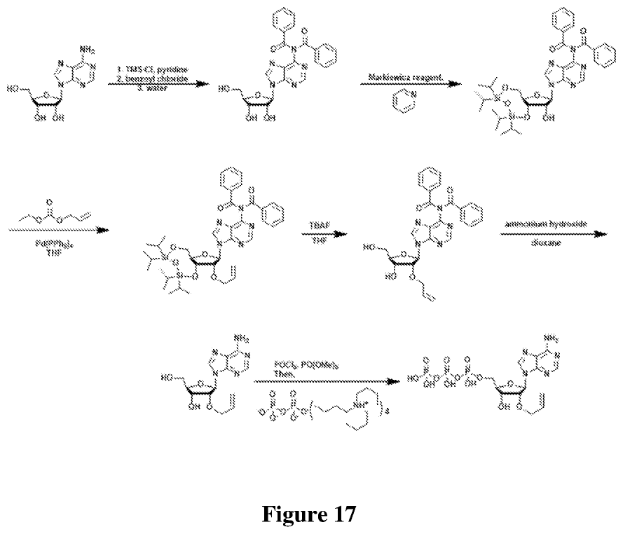

A major contributing factor to the overall cost of controlled enzymatic RNA oligonucleotide synthesis is the oligonucleotide initiator sequence. In previous examples of bulk synthesis, the oligonucleotide initiator sequence is consumed and typically non-reusable. Additionally, it is difficult to remove the oligonucleotide initiator sequence from the final product if desired. To overcome this problem, the site-specific cleavage of riboinosines (rI) and/or deoxyinosines (dI) in single stranded RNA, DNA, or combination thereof, by Endonuclease V can be used to remove unwanted initiator sequence from the final oligonucleotide product. Endonuclease V is highly specific for riboinosines (rI) and deoxyinosines (dI) and will not destroy other bases in the oligonucleotide initiator sequence. Expanding this concept to solid-phase oligonucleotide synthesis, which more is conducive for long RNA oligonucleotide synthesis and industrial relevant synthesis scales in comparison to bulk solution synthesis, a reusable set of beads, wells, slides, etc. can be produced for repeated, and potentially unlimited, synthesis runs. A brief overview of this process is given in FIG. 15A. First, a solid support is covalently derivatized with an appropriate linker (such as a long PEG chain) bound to an initiator oligonucleotide containing a riboinosine (rI) or deoxyinosine (dI), preferably at the 3′-terminus. Solid-phase enzymatic RNA oligonucleotide synthesis is conducted to produce the desired product and then Endonuclease V is allowed to incubate with the full oligonucleotide (initiator+product). This will cleave the oligonucleotide product from the solid-support, leaving the riboinosine (rI) or deoxyinosine (dI) intact on the solid-support to be reused for future synthesis reaction. Endonuclease V cleaves two bases downstream of the riboinosine (rI) or deoxyinosine (dI), so it is important to take into consideration when designing the desired oligonucleotide to be synthesized. The final product is also 5′-phosphorylated, which can be easily removed using a phosphatase or it can be used for other molecular biology or sequencing applications. In some cases, S. pombe poly(U) polymerase can be used to introduce the riboinosine (rI) to the 3′-terminus of the anchored initiator oligonucleotide using 2′-O-allyl versions of this nucleobase.

In order to determine if this scheme works as intended, an initiator oligonucleotide was synthesized with a 5′-amine group and a deoxyinosine (dI) that would yield two equally sized fragments under Endonuclease V digestion. The initiator oligonucleotide was anchored onto the surface of amine functionalized silica beads by introducing a dual-NHS-PEG9 linker that would react with the 5′-amine of the oligonucleotide and the amine on the silica beads. Derivatized beads were allowed to incubate with Endonuclease V (expressed and purified as described in the materials and methods section) for 1 hour at 37° C. Additionally, the same digestion reaction was conducted in bulk phase for comparison. For both solid-phase and bulk digestion reactions, control samples were put into place which were reactions that did not contain Endonuclease V. After the 1-hour incubation, digestion reactions were analyzed using a 15% TBE-urea gel under denaturing conditions. Gels were stained with 1×SYBR GelStar nucleic acid stain for 15 minutes shaking at room temperature. It was observed that Endonuclease V worked as intended producing digestion fragments in both the bulk and solid-phase reactions (FIG. 15B). For the bulk control reaction, a full-length, undigested fragment is observed. No discernable fragments are observed for the solid-phase control digestion reaction suggesting that the oligonucleotide remained intact on the surface of the silica beads. This result is additionally important as this verifies the covalently bound oligonucleotide does not leech from the surface.

To demonstrate that the described solid-support system is functional for enzymatic synthesis and that initiator oligonucleotide is reusable via the deoxyinosine nucleobase remaining intact on the surface post Endonuclease V digestion, washed silica beads harboring digested initiator oligonucleotide were incubated with S. pombe poly(U) polymerase and natural rNTPs (uncontrolled extension) as well as the 2′-O-allyl-ATP reversible terminator (controlled extension) using optimized reaction conditions. For comparison, beads with newly anchored, undigested initiator oligonucleotide were extended similarly. All beads were then washed with 10 mM Tris-HCl (pH 6.5) and allowed to incubate in the presence of Endonuclease V for 1 hour at 37° C. The efficiency of extension and cleavage from the surface was then analyzed using a 15% TBE-urea gel under denaturing conditions and stained with 1×SYBR GelStar nucleic acid stain for 15 minutes shaking at room temperature. The gel indicated positive extension and cleavage for both the reused and newly derivatized beads under all conditions (FIG. 15C). As a final demonstration for the viability, the controlled synthesis of an (n+2) product using the covalently bound, Endonuclease V cleavable initiator oligonucleotide with the 2′-O-allyl-ATP reversible terminator nucleoside triphosphate was performed with S. pombe poly(U) polymerase. Extension reactions were supplemented with 1 mM of nucleotide and incubated at 37° C. for 15 minutes. Deblocking reactions were performed at 50° C. for 10 minutes. Beads were washed with 10 mM Tris-HCl (pH 6.5). Endonuclease V cleavage was performed using the appropriate buffer for 1 hour at 37° C. and immediately run on the denaturing gel. The control reaction that was extended to an (n+2) and incubated in the presence of Endonuclease V, but contained an anchored Cy5 initiator oligonucleotide that did not bear a riboinosine (rI) or deoxyinosine (dI). This was used to demonstrate that the oligonucleotide did not leech during Endonuclease V cleavage. The beads with the Cy5 initiator oligonucleotide remaining visibly blue after each synthesis cycle.

### 14. Development of 3′-Blocked Reversible Terminator Nucleotides and Use in Oligonucleotide Synthesis

New nucleoside triphosphates for the enzymatic synthesis of RNA oligonucleotides and modified oligonucleotides have been developed. RNA oligonucleotides and modified oligonucleotides can be used in various applications including oligonucleotide therapeutics. Nucleoside triphosphates are reversibly terminated with a blocking group at the 3′-position of the sugar ring, conferring only (n+1) extension of the growing oligonucleotide; extension reactions do not produce a free hydroxyl group (—OH) at the 3′-position where further extension may be possible. The blocking group can be removed with a mild, biocompatible deprotection agent. This strategy compliments virtual blocking at the 2′ position or base where the growing oligonucleotide is sterically blocked rather than chemically blocked (these are also known as “virtual terminators”).

In some instances, the 3′-blocking strategy requires a compatible enzyme (e.g., mutagenesized poly(U) polymerase described herein), that accommodates the blocking chemical domain. There are a number of chemical domains that can be used for the 3′-blocking strategy as listed below. Some examples include, but are not limited to, 3′-O-allyl triphosphates (3′-O-allyl-NTP) and 3′-O-azidomethyl triphosphates (3′-O-azidomethyl-NTP).

The new 3′-reversibly terminated nucleoside triphosphates can have the advantage of including multiple modifications that confer therapeutic or other functional value to the overall oligonucleotide. Nucleoside triphosphates may have single or multiple modifications in addition to the 3′-reversible terminating group. Modifications can be introduced site-specifically into the oligonucleotide, without additional protecting groups. These nucleoside triphosphates require a compatible enzyme for their incorporation, which may hold a unique sequence or set of mutant codons for each modified nucleotide used. Modifications manifest as chemical handles, ligand binding domains, a way to confer oligonucleotide nuclease resistance, stereopure thiophosphonates oligonucleotides, a way to confer a propensity to form desired oligonucleotide secondary structure, or a way to confer resistance to form undesired oligonucleotide secondary structure, etc. This includes:

i. Modifications to the 2′-domain of the furanose ring, which may be, but limited to, a hydroxyl (—OH), hydrogen (—H), fluoro (—F), amine (—NH₃), azido (—N₃), thiol (—SH), methoxy (—OCH₃), methoxyethanol (—OCH₂CH₂OCH₃), redox-active, fluorogenic or intrinsically fluorescent moieties, natural and non-natural amino acids, peptides, proteins, mono- or oligosaccharides, functional/ligand binding glycans, and large/bulky groups such as poly-ethene-glycol (PEG).

ii. Modifications to the alpha (α) phosphate of the triphosphate, where either a thiophosphonate R or S isomer is site-specifically introduced into the oligonucleotide to produce a stereopure oligonucleotide.

iii. Modifications to the beta (β) and gamma (γ) phosphates of the triphosphate: where either and/or both modifications confer value to the enzymatic oligonucleotide synthesis scheme; for example, to prevent or limiting unwanted pyrophosphorolysis as a consequence of pyrophosphate generation,

iv. Modifications to the furanose ring, which may be, but not limited to, replacing the ring's oxygen with a sulfur or introducing a bridge between the 2′-oxygen and the 4′-carbon that limits ring conformation.

v. Modifications to the nucleobase, where the base is natural or non-natural pyrimidine or purine, and may include, but not limited to, N¹-methyl-adenine, N⁶-methyl-adenine, 8′-azido-adenine, N,N-dimethyl-adenosine, aminoallyl-adenosine, 5′-methyl-urdine, pseudouridine, N¹-methyl-pseudouridine, 5′-hydroxy-methyl-uridine, 2′-thio-uridine, 4′-thio-uridine, hypoxanthine, xanthine, 5′-methyl-cytidine, 5′-hydroxy-methyl-cytidine, 6′-thio-guanine, and N⁷-methyl-guanine.

At the completion of synthesis, oligonucleotides may be irreversibly capped with a final 3′-blocked nucleoside triphosphate that may confer further functional or therapeutic value. This also may require the use of a compatible enzyme (e.g., mutated poly(U) polymerase enzyme) and may be a modification group described herein. Additionally, both the 3′- and 2′-domains for the furanose ring may be irreversibly blocked with the same or different groups.

In addition to mono-nucleoside triphosphate, di-nucleoside triphosphates, tri-nucleoside triphosphates, and N-nucleoside triphosphates (where N=a triphosphorylated oligonucleotide of N length) can be used as substrates for incorporation using a compatible enzyme catalyst, effectively making an oligonucleotide ligase.

In some cases, the addition of a new nucleoside triphosphate may introduce a cleavable handle that can be acted upon by chemical or biological means for post-synthesis processing and purification. For example, oligonucleotides bearing a hypoxanthine (inosine) group may be site-specifically cleaved by Endonuclease V. This is particularly useful for solid-phase synthesis of oligonucleotides, where the bound oligonucleotide initiator can be reused indefinitely.

Enzymatic Oligonucleotide Synthesis with Wild-Type and Mutated Poly(N) Polymerases

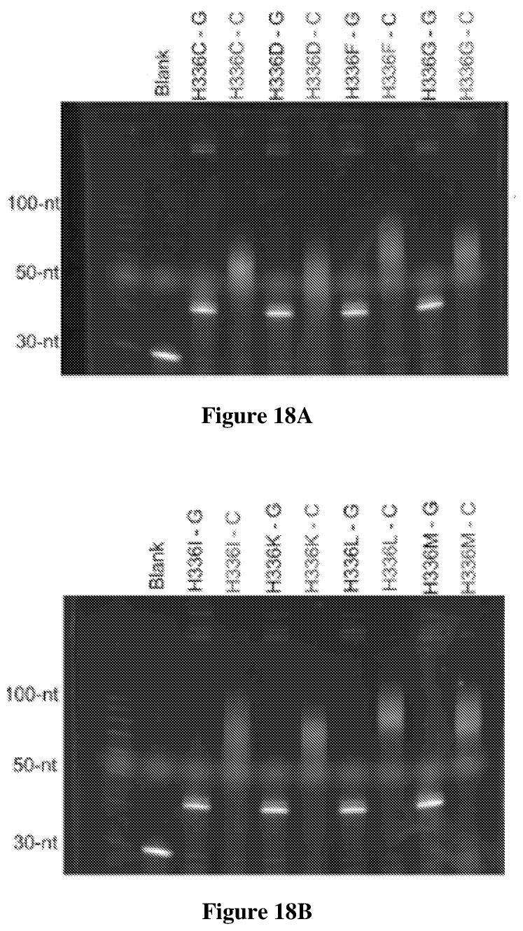

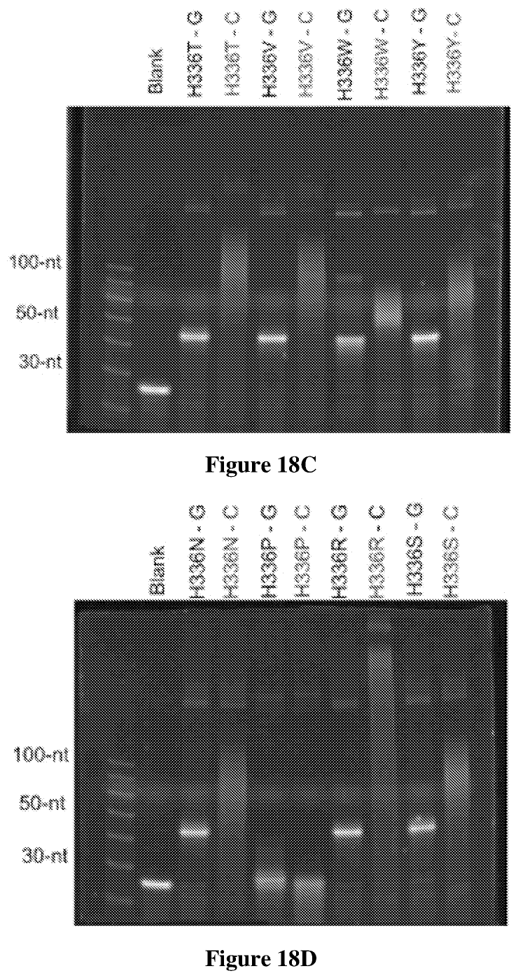

Gel electrophoresis analysis of H336 mutants' capacity to incorporate the natural nucleotide GTP—“G” and CTP—“C” is shown in FIGS. 18A to 18D. Blank reactions were supplemented with all components except enzyme and nucleotide. All reactions were incubated with 1 mM nucleotide, 5 pmol initiator oligonucleotide, and 1 μg of enzyme for 30 minutes at 37° C. A 15% TBE-Urea denaturing gel was used for extension reaction analysis.

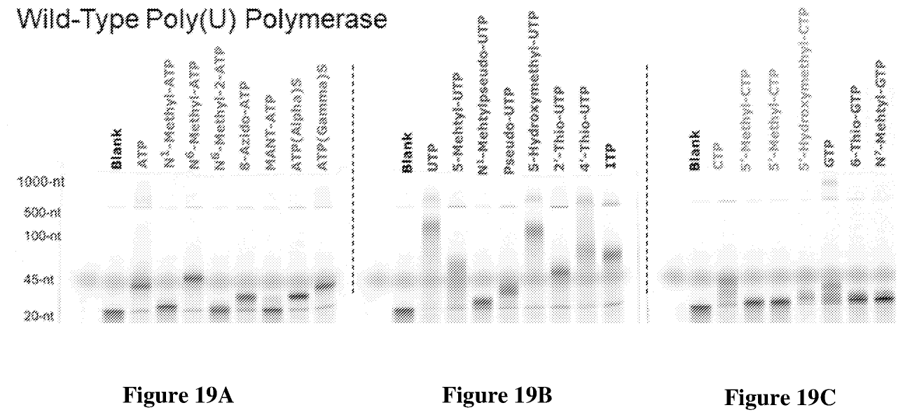

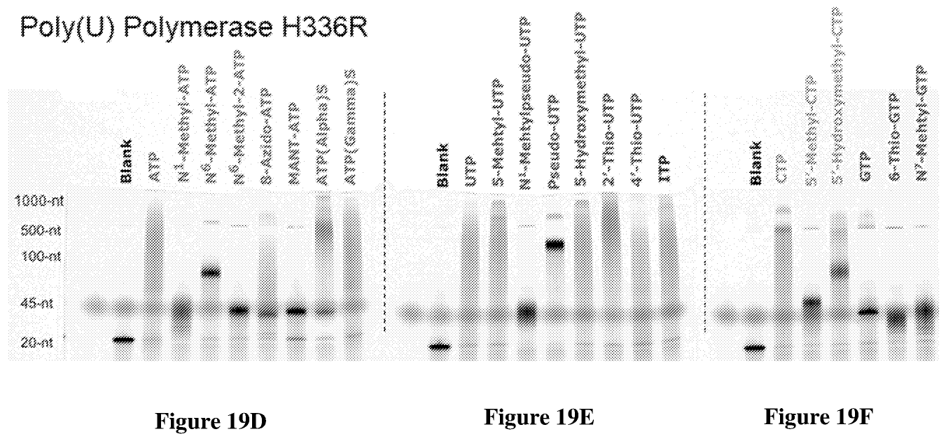

Gel electrophoresis analysis of poly(U) polymerase mutant H336R capacity to incorporate an array of natural and analogue nucleotides in comparison to the wild-type poly(U) polymerase is shown in FIGS. 19A to 19F. FIGS. 19A, 19D depict extension results for ATP based nucleotides for the wild-type and H336R mutant, respectively. FIGS. 19B, 19E depict extension results for UTP and ITP based nucleotides for the wild-type and H336R mutant, respectively. FIGS. 19C, 19F depict extension results for CTP and GTP based nucleotides for the wild-type and H336R mutant, respectively. All reactions were incubated with 1 mM nucleotide, 5 pmol initiator oligonucleotide, and 1 μg of enzyme for 30 minutes at 37° C. A 15% TBE-Urea denaturing gel was used for extension reaction analysis.

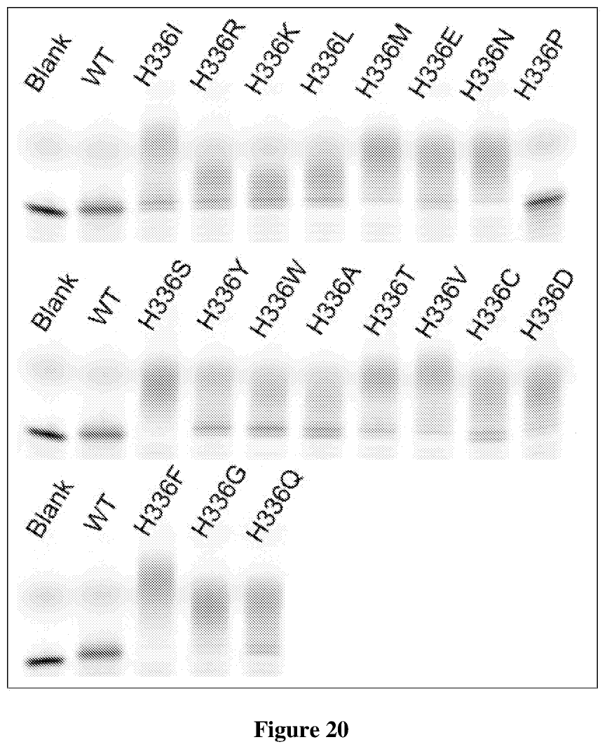

Uncontrolled incorporation of 2′-methoxy-adenosine triphosphate (2′-O-Me-ATP) by various S. pombe poly(U) polymerase mutants, specifically at position H336 is shown in FIG. 20; single mutants are shown here in comparison to the wild-type (WT). Blank reactions contain all components except enzyme. Samples were analyzed with a 15% TBE-urea gel under denaturing conditions.

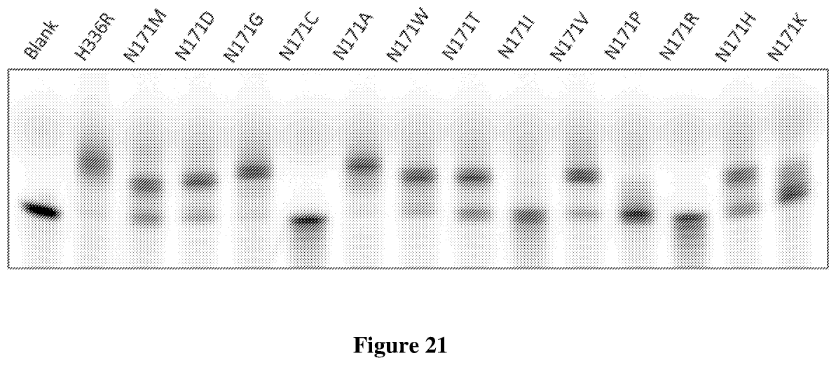

Uncontrolled incorporation of 2′-fluoro-adenosine triphosphate (2′-F-ATP) by various S. pombe poly(U) polymerase mutants, specifically at position N171 is shown in FIG. 21; single mutants are shown here in comparison to mutant H336R. Blank reactions contained all reaction components except enzyme. Samples were analyzed with a 15% TBE-urea gel under denaturing conditions.

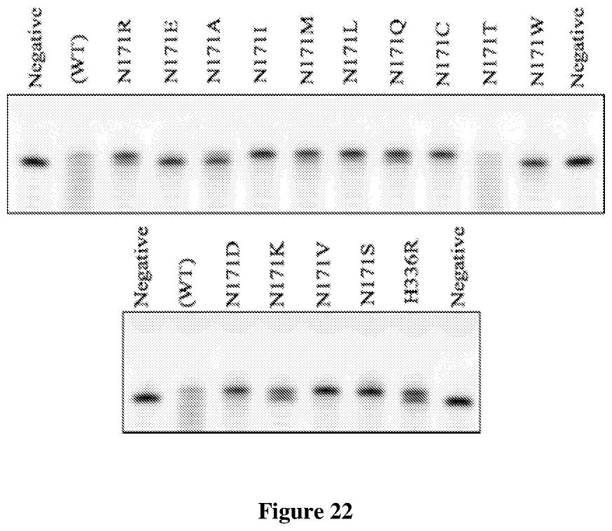

Controlled incorporation (capping) of 3′-methoxy-adenosine triphosphate (3′-O-Me-ATP) by various S. pombe poly(U) polymerase mutants, specifically at position N171 is shown in FIG. 22; single mutants are shown here in comparison to mutant H336R and the wild-type. Top band indicates (n+1) product. Note: the wild-type sample indicates positive incorporation, however severe pyrophosphorolysis occurs. Negative reactions contained all reactions components except enzyme. Samples were analyzed using a 15% TBE-Urea gel under denaturing conditions.

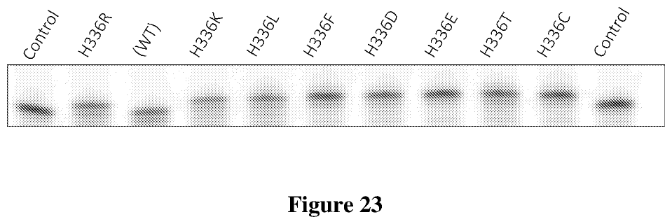

Controlled incorporation of the reversible terminator 3′-O-allyl Adenosine Triphosphate (3′-(O-allyl)-ATP) by various S. pombe Mutants is shown in FIG. 23. Negative reactions contained all reactions components except enzyme. Samples were analyzed using a 15% TBE-Urea gel under denaturing conditions.

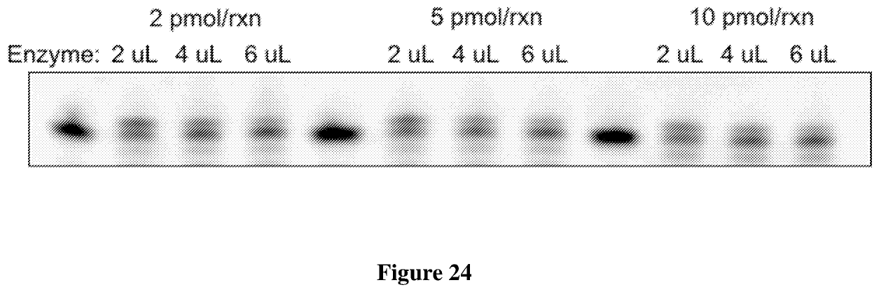

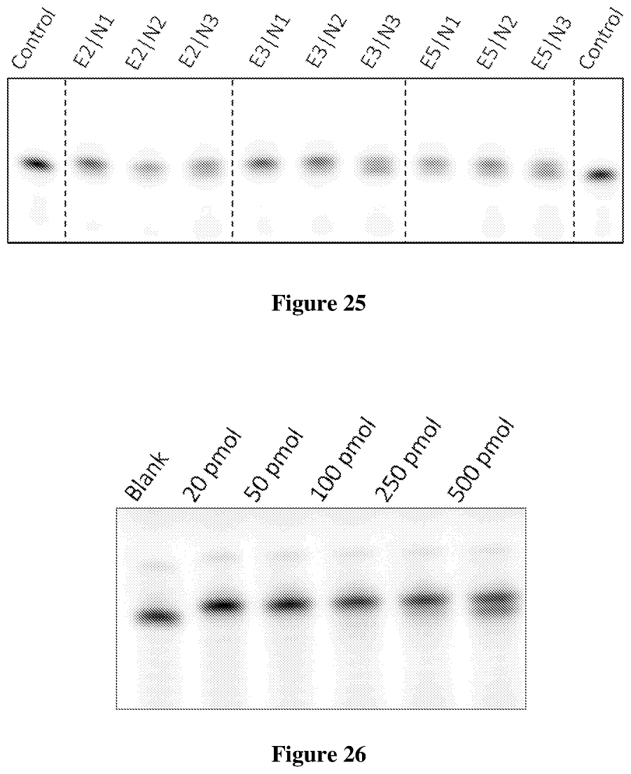

Controlled incorporation of the reversible terminator 3′-O-allyl carbonate deoxyadenosine triphosphate (3′-(0-allyl carbonate)-dATP) by the poly(U) polymerase double mutant H336R-N171A is shown in FIG. 24. Gel image indicates varying input amounts of initiator oligonucleotide (2 pmol/r×n, 5 pmol/r×n, and 10 pmol/r×n) with increasing amounts of purified enzyme stock (2 μL, 4 μL, and 6 μL). Top band indicates (n+1) product. Blank reactions contain all components except enzyme. Samples were analyzed with a 15% TBE-urea gel under denaturing conditions.

Reaction calibration assessment of purified poly(U) polymerase stock H336R with reversible terminator 3′-O-allyl Adenosine Triphosphate 3′-(O-allyl)-ATP) is shown in FIG. 26. Gel indicates response of (n+1) extension given increasing input amounts of initiator oligonucleotide. Reactions are supplemented with 1 mM reversible terminator nucleotide and 1 μL of purified enzyme stock. Reactions were incubated for 5 minutes at 37° C. Top band indicates (n+1) product. Blank reactions contain all components except enzyme. Samples were analyzed with a 15% TBE-urea gel under denaturing conditions. This is an example of reaction scalability.

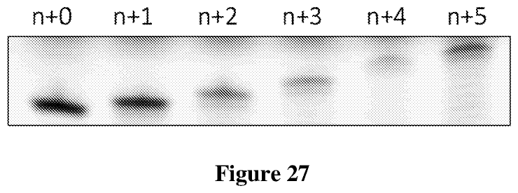

Demonstration of controlled enzymatic synthesis is shown in FIG. 27 using poly(U) polymerase mutant H336R with the reversible terminator with 3′-O-allyl Adenosine Triphosphate (3′-O-allyl-ATP) in bulk solution. Shown here is an (n+5) synthesis in bulk solution. Post-synthesis, reactions were analyzed using a 15% TBE-urea gel under denaturing conditions.

### 3′-Reversible Terminator Structures & Synthesis

Exemplary structures of 3′-reversible terminator nucleotides for enzymatic incorporation are shown in FIG. 28. Various examples of protecting groups for the 3′ hydroxy. As labeled, these may be removed through redox chemistry, light, fluoride anions and catalysts.

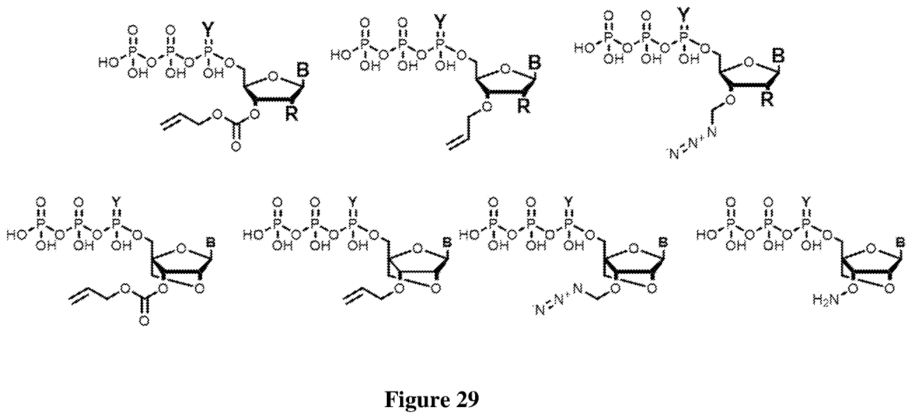

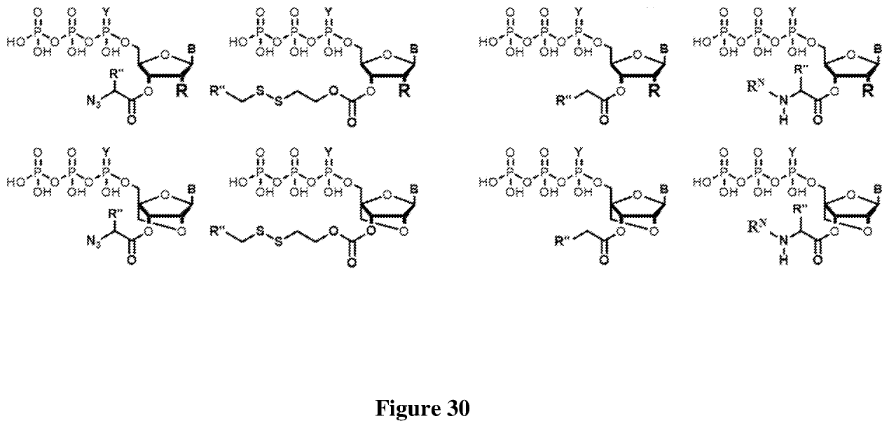

Select 3′ protecting groups where the furanyl ring bears oxygen are shown in FIGS. 29 and 30. The 2′ may be natural ribo, deoxy or various moieties that promote binding, pharmacokinetics, pharmacodynamics, general stability and probe tags.

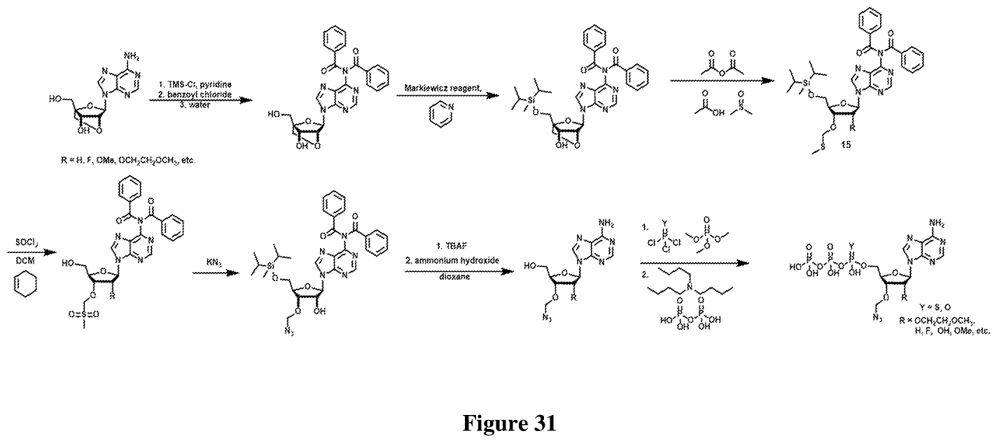

Exemplary scheme for the preparation of a 3′ azidomethyl ether for a nucleotide triphosphate is shown in FIG. 31, where the 2′ may be natural OH or various modifications such as —F, —OMe, —OCH₂CH₂CH₃ or others which prove beneficial for biological activity of target oligoes or contributing to the broader impacts of science.

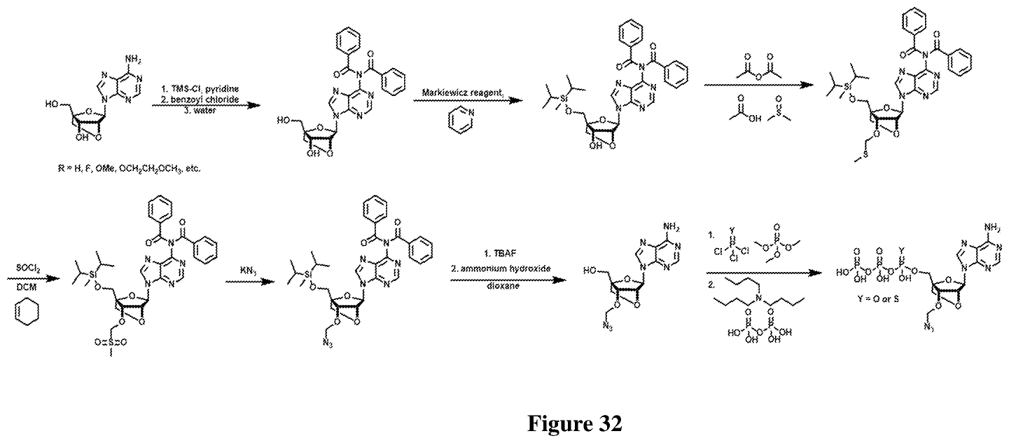

Exemplary scheme for the preparation of a 3′ azidomethyl ether for a locked nucleotide triphosphate is shown in FIG. 32.

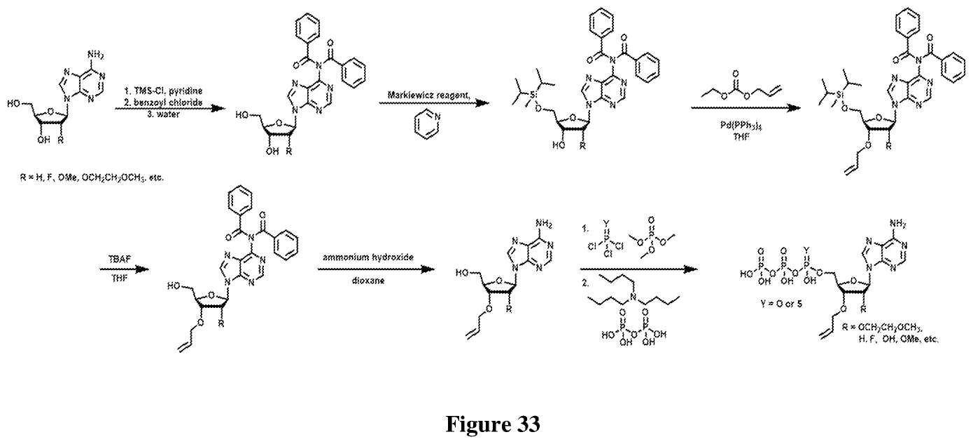

Exemplary scheme for the preparation of a 3′ allyl ether for a nucleotide triphosphate is shown in FIG. 33, where the 2′ may be natural OH or various modifications such as F, OMe, OCH2CH2CH3 or others which prove beneficial for biological activity of target oligoes or contributing to the broader impacts of science.

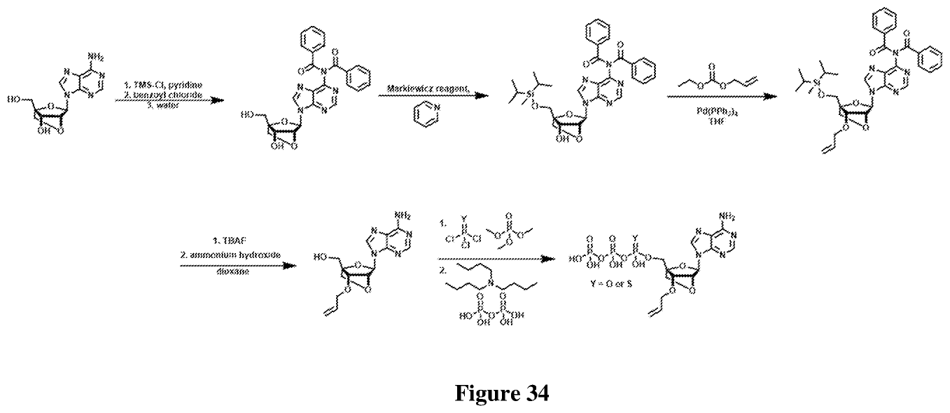

Exemplary scheme for the preparation of a 3′ azidomethyl ether for a locked nucleotide triphosphate is shown in FIG. 34.

Sequences
Human polymerase Mu R387K
(SEQ ID NO: 1)
MLPKRRRARVGSPSGDAASSTPPSTRFPGVAIYLVEPRMGRSRRAFLTGL
ARSKGFRVLDACSSEATHVVMEETSAEEAVSWQERRMAAAPPGCTPPALL
DISWLTESLGAGQPVPVECRHRLEVAGPRKGPLSPAWMPAYACQRPTPLT
HHNTGLSEALEILAEAAGFEGSEGRLLTFCRAASVLKALPSPVTTLSQLQ
GLPHFGEHSSRVVQELLEHGVCEEVERVRRSERYQTMKLFTQIFGVGVKT
ADRWYREGLRTLDDLREQPQKLTQQQKAGLQHHQDLSTPVLRSDVDALQQ
VVEEAVGQALPGATVTLIGGERRGKLQGHDVDFLITHPKEGQEAGLLPRV
MCRLQDQGLILYHQHQHSCCESPTRLAQQSHMDAFEKSFCIFRLPQPPGA
AVGGSTRPCPSWKAVRVDLVVAPVSQFPFALLGWTGSKLFQRELRRFSRK
EKGLWLNSHGLFDPEQKTFFQAASEEDIFRHLGLEYLPPEQRNA
Saccharomyces cerevisiae poly(A) polymerase
(SEQ ID NO: 2)
MSSQKVFGITGPVSTVGATAAENKLNDSLIQELKKEGSFETEQETANRVQ
VLKILQELAQRFVYEVSKKKNMSDGMARDAGGKIFTYGSYRLGVHGPGSD
IDTLVVVPKHVIREDFFTVEDSLLRERKELDEIAPVPDAFVPIIKIKESG
ISIDLICARLDQPQVPLSLTLSDKNLLRNLDEKDLRALNGTRVTDEILEL
VPKPNVFRIALRAIKLWAQRRAVYANIFGFPGGVAWAMLVARICQLYPNA
CSAVILNRFFIILSEWNWPQPVILKPIEDGPLQVRVWNPKIYAQDRSHRM
PVITPAYPSMCATHNITESTKKVILQEFVRGVQIINDIFSNKKSWANLFE
KNDEFFRYKFYLEITAYTRGSDEQHLKWSGLVESKVRLLVMKLEVLAGIK
IAHPFTKPFESSYCCPTEDDYEMIQDKYGSHKTETALNALKLVTDENKEE
ESIKDAPKAYLSTMYIGLDFNIENKKEKVDIHIPCTEFVNLCRSFNEDYG
DHKVFNLALRFVKGYDLPDEVFDENEKRPSKKSKRKNLDARHETVKRSKS
DAASGDNINGTTAAVDVN
Schizosaccharomyces pombe poly(U) polymerase
(SEQ ID NO: 3)
MNISSAQFIPGVHTVEEIEAEIHKNLHISKSCSYQKVPNSHKEFTKFCYE
VYNEIKISDKEFKEKRAALDTLRLCLKRISPDAELVAFGSLESGLALKNS
DMDLCVLMDSRVQSDTIALQFYEELIAEGFEGKFLQRARIPIIKLTSDTK
NGFGASFQCDIGFNNRLAIHNTLLLSSYTKLDARLKPMVLLVKHWAKRKQ
INSPYFGTLSSYGYVLMVLYYLIHVIKPPVFPNLLLSPLKQEKIVDGFDV
GFDDKLEDIPPSQNYSSLGSLLHGFERFYAYKEEPREKVVTERRPDGYLT
KQEKGWTSATEHTGSADQIIKDRYILAIEDPFEISHNVGRTVSSSGLYRI
RGEFMAASRLLNSRSYPIPYDSLFEEAPIPPRRQKKTDEQSNKKLLNETD
GDNSE

### Materials & Methods

### Enzyme Expression and Purification

The primary sequences of wild-type or mutant enzymes of interest were codon optimized for E. coli expression using a custom optimization algorithm and ordered as gBlocks® (IDT) with 20-nt overlap sequences for Gibson Assembly into the pET-28-c-(+) His-tag expression vector (EMD Millipore 69866-3). Using forward and reverse primers from IDT, the gBlocks® were PCR amplified with Phusion High Fidelity (HF) polymerase (NEB M05030). The PCR thermocycling was performed as follows: initial denature for 98° C. for 30 seconds, denature at 98° C. for 10 seconds, anneal at 68° C. for 10 seconds, and extend at 72° C. for 60 seconds for 18 cycles before a final extension of 5 minutes at 72° C. PCR reactions were purified and concentrated using a QIAquick PCR Purification Kit (Qiagen 28106).

The pET-28-c-(+) expression vector was prepared for gBlocks® insertion by digesting the circular DNA with 40U of NDeI (NEB R0111) per 500 ng vector at 37° C. for 90 minutes. The linear DNA was separated from undigested material with 2% agarose gel electrophoresis and extracted by incubating agarose containing the bands corresponding to the linear DNA in Buffer QG (Qiagen 19063) at 55° C. rotating at 1000 RPM for 2 hours. The resultant mixture was cleaned and concentrated with the QIAquick PCR Purification Kit. The PCR amplified insert and vector sequences were combined at a ratio of 1:3 with 0.1 pmol of total material and assembled with Gibson Assembly Master Mix (NEB E5510S) at 50° C. for 1 hour. T7 Express chemically competent E. coli (NEB C2566I) were transformed with the fully assembled plasmid as per manufacturer's instructions and positive transformants are selected for on LB-kanamycin plates (50 ug/mL kanamycin).

Bacterial colonies were sequenced (Genewiz, T7—Forward Primer, T7 Term—Reverse Primer) and those with perfect matches were grown in liquid LB-kanamycin media (50 μg/mL kanamycin) overnight, diluted 1:400 in fresh liquid LB-kanamycin, and induced with 1 mM IPTG (Sigma 16758) at approximately OD₆₀₀=0.8. The induced liquid cultures were incubated overnight at 15° C., shaking at 250 RPM. Cultures were then pelleted at 3500×g for 10 minutes and then His-Tag purified using a HisTalon Resin Kit as per manufacturer's instructions (Clontech 635654). The eluted enzyme samples were then buffer exchanged into an optimal 2× protein storage buffer using 15-mL filter columns (Millipore) at the appropriate MWCO by centrifugation at 5000×G for 15 minutes at 4° C. This process was repeated twice. On the third spin, samples were spun for 30 minutes in order to concentrate the protein into a smaller volume.

Two small aliquots were taken for to determine the overall protein concentration using a Reducing Agent Compatible MicroBCA kit (Thermo 23252) and to determine the size of the His-Tag purified protein using a 16% Tris-Gly denaturing gel (Thermo XP00165) with a 10-250 kDa protein ladder (Thermo 26619). Post gel-electrophoresis, gels were stained with Coomassie Orange Fluor (Thermo C33250) for 20 minutes at room temperature under gentle agitation and visualized using a GelDoc Image Station (Biorad). The remaining concentrated stock of protein was diluted 1:2 with sterile glycerol and stored at −20° C.

### Site-Directed Mutagenesis of Target Proteins to Improve RNA Synthesis

Single or multiple amino acids may be mutagenized for improvement in either of the RNA oligonucleotide synthesis schemes by rational design or by high-throughput methods such as error-prone PCR. Plasmids carrying the target protein were harvested and purified from a sequence verified liquid bacterial cultures grown overnight in LB-kanamycin media at 37° C. using a MiniPrep Kit (Qiagen 27104). Oligonucleotide primers were ordered from IDT and were designed to PCR amplify the protein expression plasmid while simultaneously mutagenizing the plasmid at the predetermined location, yielding linearized DNA. Using the reagents from the Q5 Site-Directed Mutagenesis Kit (NEB E0554S), the protein expression plasmid was PCR amplified using the Q5 Hot Start High-Fidelity 2x Master Mix with the following thermocycling conditions: initial denature for 98° C. for 30 seconds, denature at 98° C. for 10 seconds, anneal at 68° C. for 10 seconds, and extend at 72° C. for 120 seconds for 25 cycles before a final extension of 2 minutes at 72° C. 1 μL of the resulting PCR amplification reaction was then treated with the kit's enzyme reaction cocktail to re-circularize the protein expression plasmid while digesting away the unsubstituted plasmid sequences remaining in the reaction mixture. After bacterial transformation and sequence verification, colonies with perfect sequence matches were used to express and analyze the site-directed mutant protein with methods previously outlined. The resultant purified mutagenized protein was concentrated and buffer exchanged into the appropriate 2× storage buffer as previously mentioned and diluted 1:2 with sterile glycerol and stored at −20° C.

Initial Activity Screen with Natural rNTPs

Expressed proteins with terminal transferase activity were screened by determining the rate of RNA generation in terms of total RNA concentration and the length/distribution of RNA produced by the protein after incubation with natural rNTPs. In order to measure the rate of RNA generation, a 10 μL bulk extension reaction consisting of 10 pmol of a short 5′-Cy5 labeled initiator oligonucleotide (15-20-nt), 100 μM of rNTPs, 0.25 mM divalent cation cofactor (such as Co²⁺, Mg²⁺, Mn²⁺, Zn²⁺, or combinations thereof), 1x Reaction Buffer, 1x SYBR Dye (GelStar (Lonza 50535), Qubit ssDNA Dye (Thermo Q10212), or SYBR Green II RNA gel stain (Thermo S7564), and 1 μL of purified enzyme was monitored on a plate reader (EX:598 nm, EM:522 nm) over 30 minutes at 37° C., taking signal reads every 1 minute in triplicate (N=3). Using a custom R script, the rate of RNA generation and, subsequently enzyme initial activity (Vₒ), was determined from the slope of the best fit curve of the average RFU plotted as a function of time. The length of the RNA produced in these reactions was determined by comparing products to a 100-nt ssDNA ladder (Simplex Biosciences) using a 15% TBE-Urea denaturing gel (Thermo EC6885) following the manufacturer's protocol. Approximately 8 μL of the initial activity screen reaction volume was loaded onto the gels and run at 185V for 60 minutes unless otherwise specified. Gels were then stained with a solution of 1× GelStar Nucleic Acid stain or SYBR Green II RNA gel stain for 15 minutes with gentle agitation. The resultant gel was then imaged on a Typhoon FLA 9500 system (GE Healthcare Life Sciences) using imaging parameters for SYBR Gold. For extension reactions using initiator oligonucleotides labeled with a 5′-fluorophore such as FAM, Cy5, Cy3, etc., gels were not stained and imaged directly using the appropriate parameters.

Enzyme Activity Assay—Uncontrolled Extension with Natural & Analogue Nucleotides

Uncontrolled extension reactions were comprised of 5 pmol of initiator oligonucleotide, 1 mM natural or analogue nucleotide, 1× poly(U) polymerase reaction buffer (10 mM NaCl, 10 mM Tris-HCl, 10 mM MgCl₂, 1 mM DTT, pH 7.9 at 25 C), and 1 μg of purified enzyme. Natural and analogue nucleotides were either purchased from commercial sources or custom synthesized in-house. Reactions were incubated at 37° C. for 30 minutes and immediately analyzed by gel electrophoresis using a 15% TBE-Urea denaturing gel (Thermo EC6885) as per manufacturer's instructions. The length of the oligonucleotide produced in these reactions was determined by comparing products to a 100-nt ssDNA ladder (Simplex Biosciences). Gels were then stained with a solution of 1x GelStar Nucleic Acid stain or SYBR Green II RNA gel stain for 15 minutes with gentle agitation. The resultant gel was then imaged on a Typhoon FLA 9500 system (GE Healthcare Life Sciences) using imaging parameters for SYBR Gold. For extension reactions using initiator oligonucleotides labeled with a 5′-fluorophore such as FAM, Cy5, Cy3, etc, gels were not stained and imaged directly using the appropriate parameters.

Enzyme Activity Assay—Controlled Extension with Natural & Analogue Reversible Terminator Nucleotides

Controlled extension reactions were comprised of 5 pmol of initiator oligonucleotide, 1 mM blocked reversible terminator nucleotides, 1× poly(U) polymerase reaction buffer (10 mM NaCl, 10 mM Tris-HCl, 10 mM MgCl₂, 1 mM DTT, pH 7.9 at 25 C), and 1 μg of purified enzyme. Reactions were incubated at 37° C. for 1 minute and immediately analyzed by gel electrophoresis using a 15% TBE-Urea denaturing gel (Thermo EC6885) as per manufacturer's instructions. Success of the (N+1) event was determined by running a blank extension reaction in which no nucleotide or enzyme was supplemented. Gels were then stained with a solution of 1× GelStar Nucleic Acid stain or SYBR Green II RNA gel stain for 15 minutes with gentle agitation. The resultant gel was then imaged on a Typhoon FLA 9500 system (GE Healthcare Life Sciences) using imaging parameters for SYBR Gold. For extension reactions using initiator oligonucleotides labeled with a 5′-fluorophore such as FAM, Cy5, Cy3, etc, gels were not stained and imaged directly using the appropriate parameters.

### Enzyme Activity Assay—Uncontrolled or Controlled Extension Reactions on a Surface

Both uncontrolled and controlled extension reactions can be performed using surface bound initiator oligonucleotide. The surface bound initiator oligonucleotide was obtained from IDT with a 5′-amine C₆ spacer group and an internal Cy5 fluorophore. This oligonucleotide was then biotinylated and PEG-ylated using an EZ Link NHS-PEG12-Biotin kit (Thermo A35389) as per manufacturer's instructions and then clean and concentrated using an Oligonucleotide Clean and Concentrator Spin-column Kit (Zymo D4060). Derivatized initiator oligonucleotide was then bound to the surface of a streptavidin coated PCR plate (BioTez, Germany) by incubating oligonucleotide in 2× Binding and Wash buffer (10 mM Tris-HCl, 2M NaCl, 1 mM EDTA, pH 7.5 at 25° C.) for 1 hour with gentle agitation (300 RPM) in the plate wells. Wells were then aspirated and then washed once with 1× Binding and Wash Buffer. Extension reaction cocktails were made up as previously described and incubated with surface bound oligonucleotide for a predetermined time (30 minutes for uncontrolled & 1 minutes for controlled) shaking at 900 RPM at 37° C. Wells were then washed again using 1× Binding and Wash Buffer. To remove the extended oligonucleotide from the surface, wells were incubated with stripping solution (95% formamide, 10 mM EDTA, pH 6.0 at 25° C.) at 65° C. for 5 minutes. Oligonucleotide suspended in the stripping solution were then cleaned and purified using an oligonucleotide spin-column and eluted into 6 μL diH₂0. Surface extension reactions were then analyzed using gel electrophoresis as previously described.

### REFERENCES

Agrawal, Neema, P. V. N. Dasaradhi, Asif Mohmmed, Pawan Malhotra, Raj K. Bhatnagar, and Sunil K. Mukherjee. 2003. “RNA Interference: Biology, Mechanism, and Applications.” Microbiology and Molecular Biology Reviews: MMBR 67 (4): 657-85.
Anavy, L., Vaknin, I., Atar, O., Amit, R. & Yakhini, Z. Data storage in DNA with fewer synthesis cycles using composite DNA letters. Nat. Biotechnol. (2019). doi:10.1038/s41587-019-0240-x
Andrade, Paula, Maria Jos6 Martin, Raquel Juirez, Francisco López de Saro, and Luis Blanco. 2009. “Limited Terminal Transferase in Human DNA polymerase μ Defines the Required Balance between Accuracy and Efficiency in NHEJ.” Proceedings of the National Academy of Sciences of the United States of America 106 (38): 16203-8.
Balbo, P. B. & Bohm, A. Mechanism of poly(A) polymerase: structure of the enzyme-MgATP-RNA ternary complex and kinetic analysis. Structure 15, 1117-1131 (2007).
Bard, J., A. M. Zhelkovsky, S. Helmling, T. N. Earnest, C. L. Moore, and A. Bohm. 2000. “Structure of Yeast poly(A) polymerase Alone and in Complex with 3′-dATP.” Science 289 (5483): 1346-49.
Baronti, L., Karlsson, H., Marusic, M. & Petzold, K. A guide to large-scale RNA sample preparation. Anal. Bioanal. Chem. 410, 3239-3252 (2018).
Beaucage, S. L. & Iyer, R. P. Advances in the Synthesis of Oligonucleotides by the Phosphoramidite Approach. Tetrahedron 48, 2223-2311 (1992).
Bertocci, Barbara, Annie De Smet, Jean-Claude Weill, and Claude-Agnes Reynaud. 2006. “Nonoverlapping Functions of DNA polymerases Mu, Lambda, and Terminal Deoxynucleotidyltransferase during Immunoglobulin V(D)J Recombination in Vivo.” Immunity 25 (1): 31-41.
Bohr, H. G. et al. Electronic Structures of LNA Phosphorothioate Oligonucleotides. Mol. Ther. Nucleic Acids 8, 428-441 (2017).
Carlson, Robert. 2018. “Competition and the Future of Reading and Writing DNA.” In Synthetic Biology: Parts, Devices and Applications, edited by Christina Smolke Sang Yup Lee Jens Nielsen Gregory Stephanopoulos.
Ceze, L., Nivala, J. & Strauss, K. Molecular digital data storage using DNA. Nat. Rev. Genet. 20, 456-466 (2019).
Cho, H. D., Verlinde, C. L. M. J. & Weiner, A. M. Reengineering CCA-adding enzymes to function as (U,G)- or dCdCdA-adding enzymes or poly(C,A) and poly(U,G) polymerases. Proc. Natl. Acad. Sci. U.S.A 104, 54-59 (2007).
Cummins, L. L. et al. Characterization of fully 2′-modified oligoribonucleotide hetero- and homoduplex hybridization and nuclease sensitivity. Nucleic Acids Res. 23, 2019-2024 (1995).
Dana, H. et al. Molecular Mechanisms and Biological Functions of siRNA. Int. J. Biomed. Sci. 13, 48-57 (2017).
Deleavey, G. F. & Damha, M. J. Designing chemically modified oligonucleotides for targeted gene silencing. Chem. Biol. 19, 937-954 (2012).
Domínguez, Orlando, José F. Ruiz, Teresa Laín de Lera, Miguel García-Díaz, Manuel A. González, Tomas Kirchhoff, Carlos Martinez-A, Antonio Bernad, and Luis Blanco. 2000. “DNA polymerase Mu (Pol), Homologous to TdT, Could Act as a DNA Mutator in Eukaryotic Cells.” The EMBO Journal 19 (7): 1731-42.
Eckstein, F. Phosphorothioates, essential components of therapeutic oligonucleotides. Nucleic Acid Ther. 24, 374-387 (2014).
Fowler, Jason D., and Zucai Suo. 2006. “Biochemical, Structural, and Physiological Characterization of Terminal Deoxynucleotidyl Transferase.” Chemical Reviews 106 (6): 2092-2110.
Goyal, N. & Narayanaswami, P. Making sense of antisense oligonucleotides: A narrative review. Muscle Nerve 57, 356-370 (2018).
Hughes, Randall A., and Andrew D. Ellington. 2017. “Synthetic DNA Synthesis and Assembly: Putting the Synthetic in Synthetic Biology.” Cold Spring Harbor Perspectives in Biology 9 (1). https://doi.org/10.1101/cshperspect.a023812.
Jamsen, Joonas A., William A. Beard, Lars C. Pedersen, David D. Shock, Andrea F. Moon, Juno M. Krahn, Katarzyna Bebenek, Thomas A. Kunkel, and Samuel H. Wilson. 2017. “Time-Lapse Crystallography Snapshots of a Double-Strand Break Repair polymerase in Action.” Nature Communications 8 (1): 253.
Juarez, Raquel, Jose F. Ruiz, Stephanie A. Nick McElhinny, Dale Ramsden, and Luis Blanco. 2006. “A Specific Loop in Human DNA polymerase Mu Allows Switching between Creative and DNA-Instructed Synthesis.” Nucleic Acids Research 34 (16): 4572-82.
Just, Andrea, Falk Butter, Michelle Trenkmann, Tony Heitkam, Mario Mörl, and Heike Betat. 2008. “A Comparative Analysis of Two Conserved Motifs in Bacterial poly(A) polymerase and CCA-Adding Enzyme.” Nucleic Acids Research 36 (16): 5212-20.
Kaczmarek, James C., Piotr S. Kowalski, and Daniel G. Anderson. 2017. “Advances in the Delivery of RNA Therapeutics: From Concept to Clinical Reality.” Genome Medicine 9 (1): 60.
Khvorova, A. & Watts, J. K. The chemical evolution of oligonucleotide therapies of clinical utility. Nat. Biotechnol. 35, 238-248 (2017).
Koch, T., Shim, I., Lindow, M., Orum, H. & Bohr, H. G. Quantum mechanical studies of DNA and LNA. Nucleic Acid Ther. 24, 139-148 (2014).
Kosuri, S. & Church, G. M. Large-scale de novo DNA synthesis: technologies and applications. Nat. Methods 11, 499-507 (2014).
Kwak, Jae Eun, and Marvin Wickens. 2007. “A Family of poly(U) polymerases.” RNA 13 (6): 860-67.
Lee, H. H., Kalhor, R., Goela, N., Bolot, J. & Church, G. M. Terminator-free template-independent enzymatic DNA synthesis for digital information storage. Nat. Commun. 10, 2383 (2019).
Lunde, Bradley M., Iris Magler, and Anton Meinhart. 2012. “Crystal Structures of the Cid1 poly (U) polymerase Reveal the Mechanism for UTP Selectivity.” Nucleic Acids Research 40 (19): 9815-24.
Ma, S., Tang, N. & Tian, J. DNA synthesis, assembly and applications in synthetic biology. Curr. Opin. Chem. Biol. 16, 260-267 (2012).
Martin, Georges, Andreas Möglich, Walter Keller, and Sylvie Doublié. 2004. “Biochemical and Structural Insights into Substrate Binding and Catalytic Mechanism of Mammalian poly(A) polymerase.” Journal of Molecular Biology 341 (4): 911-25.
Martin, G., and W. Keller. 1998. “Tailing and 3′-End Labeling of RNA with Yeast poly(A) polymerase and Various Nucleotides.” RNA 4 (2): 226-30.
Milligan, J. F., Groebe, D. R., Witherell, G. W. & Uhlenbeck, O. C. Oligoribonucleotide synthesis using T7 RNA polymerase and synthetic DNA templates. Nucleic Acids Res. 15, 8783-8798 (1987).
Moon, Andrea F., Miguel Garcia-Diaz, Vinod K. Batra, William A. Beard, Katarzyna Bebenek, Thomas A. Kunkel, Samuel H. Wilson, and Lars C. Pedersen. 2007. “The X Family Portrait: Structural Insights into Biological Functions of X Family polymerases.” DNA Repair 6 (12): 1709-25.
Moon, Andrea F., Rajendrakumar A. Gosavi, Thomas A. Kunkel, Lars C. Pedersen, and Katarzyna Bebenek. 2015. “Creative Template-Dependent Synthesis by Human polymerase Mu.” Proceedings of the National Academy of Sciences of the United States of America 112 (33): E4530-36.
Moon, Andrea F., John M. Pryor, Dale A. Ramsden, Thomas A. Kunkel, Katarzyna Bebenek, and Lars C. Pedersen. 2014. “Sustained Active Site Rigidity during Synthesis by Human DNA polymerase μ.” Nature Structural & Molecular Biology 21 (3): 253-60.
“Structural Accommodation of Ribonucleotide Incorporation by the DNA Repair Enzyme polymerase Mu.” Nucleic Acids Research 2017 45 (15): 9138-48.
Motea, Edward A., and Anthony J. Berdis. 2010. “Terminal Deoxynucleotidyl Transferase: The Story of a Misguided DNA polymerase.” Biochimica et Biophysica Acta 1804 (5): 1151-66.
Munoz-Tello, Paola, Caroline Gabus, and Stéphane Thore. 2012. “Functional Implications from the Cid1 poly(U) polymerase Crystal Structure.” Structure 20 (6): 977-86.
Nick McElhinny, Stephanie A., and Dale A. Ramsden. 2004. “Sibling Rivalry: Competition between Pol X Family Members in V(D)J Recombination and General Double Strand Break Repair.” Immunological Reviews 200 (August): 156-64.
Palluk, S. et al. De novo DNA synthesis using polymerase-nucleotide conjugates. Nat. Biotechnol. 36, 645-650 (2018).
Pardi, N., Hogan, M. J., Porter, F. W. & Weissman, D. mRNA vaccines—a new era in vaccinology. Nat. Rev. Drug Discov. 17, 261-279 (2018).
Perkel, J. M. The race for enzymatic DNA synthesis heats up. Nature 566, 565 (2019).
Pratt, C. W., Voet, D. & Voet, J. G. Fundamentals of biochemistry: life at the molecular level. Jonh Wiley and Sons (2008).
Proudfoot, Nick J. 2011. “Ending the Message: poly(A) Signals Then and Now.” Genes & Development 25 (17): 1770-82.
Roychoudhury, Ranajit. 1972. “Enzymic Synthesis of polynucleotides: OLIGODEOXYNUCLEOTIDES WITH ONE 3′-TERMINAL RIBONUCLEOTIDE AS PRIMERS FOR POLYDEOXYNUCLEOTIDE SYNTHESIS.” The Journal of Biological Chemistry 247 (12): 3910-17.
Ruiz, J. F., O. Domínguez, T. Laín de Lera, M. Garcia-Díaz, A. Bernad, and L. Blanco. 2001. “DNA polymerase Mu, a Candidate Hypermutase?” Philosophical Transactions of the Royal Society of London. Series B, Biological Sciences 356 (1405): 99-109.
Ruiz, José F., Raquel Juirez, Miguel Garcia-Diaz, Gloria Terrados, Angel J. Picher, Sergio González-Barrera, Antonio R. Fernindez de Henestrosa, and Luis Blanco. 2003. “Lack of Sugar Discrimination by Human Pol μ Requires a Single Glycine Residue.” Nucleic Acids Research 31 (15): 4441-49.
Rupaimoole, R. & Slack, F. J. MicroRNA therapeutics: towards a new era for the management of cancer and other diseases. Nat. Rev. Drug Discov. 16, 203-222 (2017).
Sahin, U., Karikó, K. & Tureci, O. mRNA-based therapeutics—developing a new class of drugs. Nat. Rev. Drug Discov. 13, 759-780 (2014).
Sastre-Moreno, Guillermo, John M. Pryor, Alberto Díaz-Talavera, José F. Ruiz, Dale A. Ramsden, and Luis Blanco. 2017. “Pol Tumor Variants Decrease the Efficiency and Accuracy of NHEJ.” Nucleic Acids Research 45 (17): 10018-31.
Strauss, Benjamin, Alexander Nierth, Marco Singer, and Andres Jaschke. 2012. “Direct Structural Analysis of Modified RNA by Fluorescent in-Line Probing.” Nucleic Acids Research 40 (2): 861-70.
Thomas, C. et al. One-step enzymatic modification of RNA 3′ termini using polymerase θ. Nucleic Acids Res. 47, 3272-3283 (2019).
Uhlmann, E. & Peyman, A. Antisense oligonucleotides: a new therapeutic principle. Chem. Rev. 90, 543-584 (1990).
Watts, J. K., Deleavey, G. F. & Damha, M. J. Chemically modified siRNA: tools and applications. Drug Discov. Today 13, 842-855 (2008).
Wilson, C. & Keefe, A. D. Building oligonucleotide therapeutics using non-natural chemistries. Curr. Opin. Chem. Biol. 10, 607-614 (2006).
Winz, Marie-Luise, Ayan Samanta, Dirk Benzinger, and Andres Jäschke. 2012. “Site-Specific Terminal and Internal Labeling of RNA by poly(A) polymerase Tailing and Copper-Catalyzed or Copper-Free Strain-Promoted Click Chemistry.” Nucleic Acids Research 40 (10): e78.
Xiong, Yong, and Thomas A. Steitz. 2004. “Mechanism of Transfer RNA Maturation by CCA-Adding Enzyme without Using an Oligonucleotide Template.” Nature 430 (7000): 640-45.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method for template-independent synthesis of an RNA oligonucleotide, the method comprising:
(a) providing an initiator oligonucleotide, wherein the initiator oligonucleotide is single-stranded RNA;
(b) providing a poly(N) polymerase;
(c) combining the initiator oligonucleotide, the poly(N) polymerase, and one or more modified nucleotides under conditions sufficient for the addition of at least one modified nucleotide to the 3′ end of the initiator oligonucleotide.

2. The method of claim 1 further comprising:
(d) repeating steps (a)-(c) until a desired RNA sequence is obtained.

3. The method of claim 1 or 2 further comprising adding one or more natural or modified nucleotides to the 3′ end of the resulting RNA oligonucleotide until a desired RNA sequence is obtained.

4. The method of any one of claims 1-3, wherein the poly(N) polymerase is a poly(U) polymerase, a poly(A) polymerase, a poly(C) polymerase, or a poly(G) polymerase; or a mutant thereof, or a homolog thereof.

5. The method of any one of claims 1-4, wherein the poly(N) polymerase is a poly(A) polymerase, or a mutant thereof, or a homolog thereof.

6. The method of claim 5, wherein the poly(A) polymerase is wild-type Saccharomyces cerevisiae poly(A) polymerase, or a mutant thereof, or a homolog thereof.

7. The method of claim 5, wherein the poly(A) polymerase is wild-type Saccharomyces cerevisiae poly(A) polymerase.

8. The method of any one of claims 1-4, wherein the poly(N) polymerase is a poly(U) polymerase, or a mutant thereof, or a homolog thereof.

9. The method of claim 8, wherein the poly(U) polymerase is wild-type Schizosaccharomyces pombe poly(U) polymerase, or a mutant thereof, or a homolog thereof.

10. The method of claim 8, wherein the poly(U) polymerase is wild-type Schizosaccharomyces pombe poly(U) polymerase.

11. The method of any one of claims 1-10, wherein at least one of the modified nucleotides is a base-modified nucleotide.

12. The method of claim 11, wherein the base-modified nucleotide comprises a modified base selected from the group consisting of 5-methylcytosine, pyridin-4-one, pyridin-2-one, phenyl, pseudouracil, 3-methyl uracil, dihydrouridine, naphthyl, aminophenyl, 5-alkylcytidines, 5-alkyluridines, 5-halouridines, 6-azapyrimidines, 6-alkylpyrimidines, propyne, quesosine, 2-thiouridine, 4-thiouridine, 4-acetyltidine, 5-(carboxyhydroxymethyl)uridine, 5-carboxymethylaminomethyl-2-thiouridine, 5-carboxymethylaminomethyluridine, β-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, β-D-mannosylqueosine, uridine-5-oxyacetic acid, 2-thiocytidine, N¹-methyl-adenine, N⁶-methyl-adenine, 8′-azido-adenine, N,N-dimethyl-adenosine, aminoallyl-adenosine, 5′-methyl-urdine, pseudouridine, N¹-methyl-pseudouridine, 5′-hydroxy-methyl-uridine, 2′-thio-uridine, 4′-thio-uridine, hypoxanthine, xanthine, 5′-methyl-cytidine, 5′-hydroxy-methyl-cytidine, 6′-thio-guanine, and N⁷-methyl-guanine, threonine derivatives, pyrimidine derivatives, and purine derivatives.

13. The method of claim 11, wherein the base-modified nucleotide is selected from the group consisting of N¹-methyladenosine-5′-triphosphate, N⁶-methyladenosine-5′-triphosphate, N⁶-methyl-2-aminoadenosine-5′-triphosphate, 5-methyluridine-5′-triphosphate, N¹-methylpseudouridine-5′-triphosphate, pseudouridine-5′-triphosphate, 5-hydroxymethyluridine-5′-triphosphate, 5-methylcytidine-5′-triphosphate, 5-hydroxymethylcytidine-5′-triphosphate, N⁷-methylguanosine-triphosphate, 8′-adizoadenisone-5′-triphosphate, inosine 5′-triphosphate, 2-thiouridine-5′-triphosphate, 6-thioguanosine-5′-triphosphate, 4-thiouridine-5′-triphosphate, and xanthosine-5′-triphosphate.

14. The method of any one of claims 1-13, wherein at least one of the modified nucleotides is a sugar-modified nucleotide.

15. The method of claim 14, wherein the sugar-modified nucleotide is modified at the 2′-position.

16. The method of claim 14, wherein the sugar-modified nucleotide is a 2′-F, 2′-O-alkyl, 2′-amino, or 2′-azido modified nucleotide.

17. The method of claim 16, wherein the sugar-modified nucleotide is a 2′-F modified nucleotide selected from the group consisting of 2′-fluoro-2′-deoxyadenosine-5′-triphosphate, 2′-fluoro-2′-deoxycytidine-5′-triphosphate, 2′-fluoro-2′-deoxyguanosine-5′-triphosphate, and 2′-fluoro-2′-deoxyuridine-5′-triphosphate.

18. The method of claim 16, wherein the sugar-modified nucleotide is a 2′-O-alkyl modified nucleotide selected from the group consisting of 2′-O-methyladenosine-5′-triphosphate, 2′-O-methylcytidine-5′-triphosphate, 2′-O-methylguanosine-5′-triphosphate, 2′-O-methyluridine-5′-triphosphate, and 2′-O-methylinosine-5′-triphosphate.

19. The method of claim 16, wherein the sugar-modified nucleotide is a 2′-O-amino modified nucleotide selected from the group consisting of 2′-amino-2′-deoxycytidine-5′-triphosphate, 2′-amino-2′-deoxyuridine-5′-triphosphate, 2′-amino-2′-deoxyadenosine-5′-triphosphate, and 2′-amino-2′-deoxyguanosine-5′-triphosphate.

20. The method of claim 16, wherein the sugar-modified nucleotide is a 2′-O-azido modified nucleotide selected from the group consisting of 2′-azido-2′-deoxycytidine-5′-triphosphate, 2′-azido-2′-deoxyuridine-5′-triphosphate, 2′-azido-2′-deoxyadenosine-5′-triphosphate, and 2′-azido-2′-deoxyguanosine-5′-triphosphate.

21. The method of any one of claims 1-20, wherein at least one of the modified nucleotide comprises a modified triphosphate.

22. The method of any one of claims 1-21, wherein at least one of the modified nucleotide is a bridged or locked nucleotide.

23. The method of any one of claims 13-22, wherein the sugar-modified nucleotide is a 2′-modified reversible terminator nucleotide.

24. The method of any one of claims 13-22, wherein the sugar-modified nucleotide is a 3′-modified reversible terminator nucleotide.

25. A method for template-independent synthesis of an RNA oligonucleotide, the method comprising:
(a) providing an initiator oligonucleotide, wherein the initiator oligonucleotide is single-stranded RNA;
(b) providing a poly(U) polymerase;
(c) combining the initiator oligonucleotide, the poly(U) polymerase, and a 2′- and/or 3′-O-protected reversible terminator nucleotide under conditions sufficient for the addition of the 2′- and/or 3′-O-protected reversible terminator nucleotide to the 3′ end of the initiator oligonucleotide;
(d) deprotecting the RNA oligonucleotide formed in step (c) at the protected 2′- and/or 3′-O-position of the 2′- and/or 3′-O-protected reversible terminator nucleotide;
(e) optionally, repeating steps (a)-(c) until a desired RNA sequence is obtained.

26. The method of claim 25, wherein the reversible terminator nucleotide is a 2′-O-protected reversible terminator nucleotide protected at the 2′-O position with an oxygen protecting group.

27. The method of claim 26, wherein the 2′-O-protected reversible terminator nucleotide is protected at the 2′-0 position with a photolabile protecting group.

28. The method of claim 26 or 27, wherein the 2′-O-protected reversible terminator nucleotide is a 2′-O-alkyl, 2′-O-silyl, 2′-O-allyl, 2′-O-azidomethyl, 2′-O-benzyl, 2′-O-coumarinyl, or a 2′-O-carbonate modified nucleotide.

29. The method of claim 28, wherein the 2′-O-protected reversible terminator nucleotide is a 2′-O-carbonate modified nucleotide selected from 2′-O-allyloxycarbonyl and 2′-O-(2-oxo-2H-chromen-4-yl)methyloxycarbonyl.

30. The method of claim 25 or 26, wherein the 2′-O-protected reversible terminator nucleotide is 2′-O-allyl-NTP or 2′-O-azidomethyl-NTP.

31. The method of any one of claims 25-30, wherein the 2′-O-protected reversible terminator nucleotide comprises a modified base moiety.

32. The method of any one of claims 25-31, wherein the 2′-O-protected reversible terminator nucleotide comprises one or more additional modifications.

33. The method of claim 25, wherein the reversible terminator nucleotide is a 3′-O-protected reversible terminator nucleotide protected at the 3′-0 position with an oxygen protecting group.

34. The method of claim 33, wherein the 3′-O-protected reversible terminator nucleotide is protected at the 3′-0 position with a photolabile protecting group.

35. The method of claim 32 or 34, wherein the 3′-O-protected reversible terminator nucleotide is a 3′-O-alkyl, 3′-O-silyl, 3′-O-allyl, 3′-O-azidomethyl, 3′-O-benzyl, 3′-O-coumarinyl, or a 3′-O-carbonate modified nucleotide.

36. The method of claim 35, wherein the 3′-O-protected reversible terminator nucleotide is a 3′-O-carbonate modified nucleotide selected from 3′-O-allyloxycarbonyl and 3′-O-(2-oxo-2H-chromen-4-yl)methyloxycarbonyl.

37. The method of claim 25 or 33, wherein the 3′-O-protected reversible terminator nucleotide is 3′-O-allyl-NTP, 3′-O-azidomethyl-NTP, 3′-O-allyl carbonate-NTP, 3′-O-allyl carbonate-dNTP, 3′-O-azidomethyl carbonate-NTP, or 3′-O-azidomethyl carbonate-dNTP.

38. The method of any one of claims 31-37, wherein the 3′-O-protected reversible terminator nucleotide comprises a modified base moiety.

39. The method of any one of claims 31-38, wherein the 3′-O-protected reversible terminator nucleotide comprises one or more additional modifications.

40. The method of claim 25, wherein the 2′- and/or 3′-O-protected reversible terminator nucleotide is of the following formula: 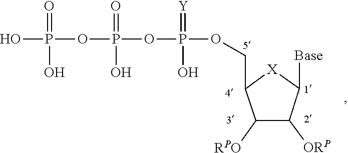
or a salt thereof, wherein:
Y is O or S;
X is O or S;
each instance of R^{P} is hydrogen, an oxygen protecting group, optionally substituted acyl, or an amino acid, or two R^{P} are joined together with the intervening atoms to form optionally substituted heterocyclyl; provided that at least one R^{P} is an oxygen protecting group, optionally substituted acyl, or an amino acid; and
“Base” is a natural or non-natural nucleotide base.

41. The method of claim 33, wherein the 3′-O-protected reversible terminator nucleotide is of the following formula: 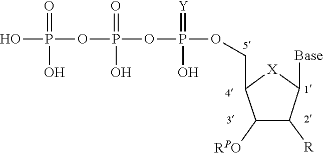
or a salt thereof, wherein:
Y is O or S;
X is O or S;
R^{P} is an oxygen protecting group, optionally substituted acyl, or an amino acid;
R is hydrogen, halogen, —CN, —NO₂, —N₃, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted hydroxyl, optionally substituted amino, or optionally substituted thiol; and
“Base” is a natural or non-natural nucleotide base.

42. The method of claim 33, wherein the 3′-O-protected reversible terminator nucleotide is of the following formula: 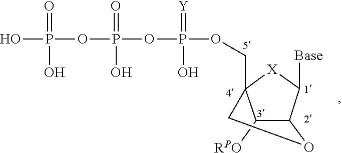
or a salt thereof, wherein:
Y is O or S;
X is O or S;
R^{P} is an oxygen protecting group, optionally substituted acyl, or an amino acid; and
“Base” is a natural or non-natural nucleotide base.

43. A method for template-independent synthesis of an RNA oligonucleotide, the method comprising:
(a) providing an initiator oligonucleotide, wherein the initiator oligonucleotide is single-stranded RNA;
(b) providing a poly(U) polymerase;
(c) combining the initiator oligonucleotide, the poly(U) polymerase, one or more nucleotides, and one or more non-hydrolyzable nucleotides under conditions sufficient for addition of at least one hydrolyzable nucleotide to the 3′ end of the initiator oligonucleotide, wherein the concentration of the non-hydrolyzable nucleotides is sufficient to inhibit the rate of addition of the one or more nucleotides by the poly(U) polymerase.

44. The method of claim 43 further comprising:
(d) repeating steps (a)-(c) until a desired RNA sequence is obtained.

45. The method of claim 43 or 44, wherein the non-hydrolyzable nucleotide comprises a modified triphosphate group.

46. The method of any one of claims 43-45, wherein the non-hydrolyzable nucleotide is selected from the group consisting of uridine-5′-[(α,β)-imido]triphosphate, adenosine-5′-[(α,β)-imido]triphosphate, guanosine-5′-[(α,β)-methyleno]triphosphate, cytidine-5′-[(α,β)-methyleno]triphosphate, adenosine-5′-[(β,γ)-methyleno]triphosphate, adenosine-5′-[(β,γ)-imido]triphosphate, guanosine-5′-[(β,γ)-imido]triphosphate, and uridine-5′-[(β,γ)-imido]triphosphate.

47. The method of claim 43 or 44, wherein the non-hydrolyzable nucleotide is a 3′-modified nucleotide.

48. The method of claim 47, wherein the non-hydrolyzable nucleotide is selected from the group consisting of 3′-O-methyladenosine-5′-triphosphate and 3′-O-methyluridine-5′-triphosphate.

49. The method of any one of claims 43-48, wherein 1-100 of the nucleotides are incorporated.

50. The method of any one of claims 43-48, wherein 1-50 of the nucleotides are incorporated.

51. The method of any one of claims 43-48, wherein 1-20 of the nucleotides are incorporated.

52. A method for the synthesis of a RNA oligonucleotide, the method comprising:
(a) providing a first oligonucleotide, wherein the first oligonucleotide comprises a 5′-triphosphate group;
(b) providing a second oligonucleotide;
(c) providing a poly(U) polymerase;
(d) combining the first and second oligonucleotides and the poly(U) polymerase under conditions sufficient for the ligation of the first oligonucleotide to the 3′ end of the second oligonucleotide.

53. The method of any one of claims 25-52, wherein the poly(U) polymerase is wild-type Schizosaccharomyces pombe poly(U) polymerase, or a mutant thereof.

54. The method of any one of claims 25-52, wherein the poly(U) polymerase is wild-type Schizosaccharomyces pombe poly(U) polymerase.

55. The method of any one of claims 1-54 further comprising a step of:
(f) performing reverse transcription on the resulting RNA oligonucleotide using a reverse transcription priming site, primer, and a reverse transcriptase enzyme, thereby producing a complementary single-stranded DNA oligonucleotide or cDNA.

56. The method of claim 55 further comprising a step of:
(g) amplifying complementary single-stranded DNA oligonucleotides or cDNA produced in step (f) with a DNA polymerase, thereby producing a double-stranded DNA.

57. The method of any one of claims 1-56, wherein step (c) is carried out in the presence of a crowding agent.

58. The method of claim 57, wherein the crowding agent is polyethylene glycol (PEG).

59. The method of any one of claims 1-58, wherein step (c) is carried out in the presence of one or more additional enzymes.

60. The method of claim 59, wherein step (c) is carried out in the presence of an additional poly(N) polymerase.

61. The method of claim 59, wherein step (c) is carried out in the presence of a yeast inorganic pyrophosphatase (PPI-ase).

62. The method of any one of claims 1-61, wherein step (c) is carried out in the presence of an RNase inhibitor.

63. The method of any one of claims 1-62, wherein step (c) is carried out in the presence of a non-hydrolyzable nucleotide.

64. The method of any one of claims 1-63, wherein the initiator oligonucleotide is covalently linked to a solid support.

65. The method of claim 64, wherein the initiator oligonucleotide is covalently linked to a solid support through a cleavable linker.

66. The method of any one of claims 1-65, wherein the initiator oligonucleotide is 5-20 nucleotides in length.

67. The method of claim 66, wherein the initiator oligonucleotide is poly-rU, poly-rC, poly-rG, or poly-rA.

68. The method of any one of claims 1-67, wherein the initiator oligonucleotide comprises a fluorophore or a handle for bioconjugation.

69. The method of any one of claims 1-68, wherein the initiator oligonucleotide comprises a primer site for reverse transcription or a primer for PCR.

70. The method of any one of claims 1-69, wherein the initiator oligonucleotide comprises a 5′ cap.

71. The method of any one of claims 1-70 further comprising a step of isolating the resulting RNA oligonucleotide.

72. The method of any one of claims 8-71, wherein the poly(U) polymerase is a mutated Schizosaccharomyces pombe poly(U) polymerase.

73. The method of claim 72, wherein the mutated Schizosaccharomyces pombe poly(U) polymerase comprises an H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W.

74. The method of claim 73, wherein the mutated Schizosaccharomyces pombe poly(U) polymerase comprises an H336R mutation.

75. The method of claim 74, wherein the mutated Schizosaccharomyces pombe poly(U) polymerase is identical to SEQ ID NO:3, but includes an H336R mutation.

76. The method of any one of claims 72-75, wherein the mutated Schizosaccharomyces pombe poly(U) polymerase comprises an N171 mutation selected from the group consisting of N171E, N171L, N171Q, N171S, N171M, N171D, N171G, N171C, N171A, N171W, N171T, N171I, N171V, N171P, N171R, N171H, and N171K.

77. The method of claim 76, wherein the mutated Schizosaccharomyces pombe poly(U) polymerase comprises an N171A mutation.

78. The method of claim 77, wherein the mutated Schizosaccharomyces pombe poly(U) polymerase is identical to SEQ ID NO:3, but includes a N171A mutation.

79. The method of any one of claims 72-78, wherein the mutated Schizosaccharomyces pombe poly(U) polymerase comprises H336 and N171 mutations.

80. The method of claim 79, wherein the mutated Schizosaccharomyces pombe poly(U) polymerase comprises H336R and N171A mutations.

81. The method of claim 80, wherein the mutated Schizosaccharomyces pombe poly(U) polymerase is identical to SEQ ID NO:3, but includes H336R and N171A mutations.

82. The method of claim 79, wherein the mutated Schizosaccharomyces pombe poly(U) polymerase comprises H336R and N171T mutations.

83. The method of claim 80, wherein the mutated Schizosaccharomyces pombe poly(U) polymerase is identical to SEQ ID NO:3, but includes H336R and N171T mutations.

84. The method of any one of claims 72-83, wherein the mutated Schizosaccharomyces pombe poly(U) polymerase comprises an T172 mutation selected from the group consisting of T172E, T172L, T172Q, T172S, T172M, T172D, T172G, T172C, T172A, T172W, T172T, T172I, T172V, T172P, T172R, T172H, and T172K.

85. The method of claim 84, wherein the mutated Schizosaccharomyces pombe poly(U) polymerase is identical to SEQ ID NO:3, but includes a T172 mutation selected from the group consisting of T172E, T172L, T172Q, T172S, T172M, T172D, T172G, T172C, T172A, T172W, T172T, T172I, T172V, T172P, T172R, T172H, and T172K.

86. The method of any one of claims 72-85, wherein the mutated Schizosaccharomyces pombe poly(U) polymerase comprises H336 and T172 mutations.

87. The method of claim 86, wherein the mutated Schizosaccharomyces pombe poly(U) polymerase comprises an H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W; and a T172 mutation selected from the group consisting of T172E, T172L, T172Q, T172S, T172M, T172D, T172G, T172C, T172A, T172W, T172T, T172I, T172V, T172P, T172R, T172H, and T172K.

88. The method of claim 87, wherein the mutated Schizosaccharomyces pombe poly(U) polymerase is identical to SEQ ID NO:3, but includes an H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W; and a T172 mutation selected from the group consisting of T172E, T172L, T172Q, T172S, T172M, T172D, T172G, T172C, T172A, T172W, T172T, T172I, T172V, T172P, T172R, T172H, and T172K.

89. The method of any one of claims 72-88, wherein the mutated Schizosaccharomyces pombe poly(U) polymerase comprises H336, N171, and T172 mutations.

90. The method of claim 85, wherein the mutated Schizosaccharomyces pombe poly(U) polymerase comprises an H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W; an N171 mutation selected from the group consisting of N171E, N171L, N171Q, N171S, N171M, N171D, N171G, N171C, N171A, N171W, N171T, N171I, N171V, N171P, N171R, N171H, and N171K; and an T172 mutation selected from the group consisting T172E, T172L, T172Q, T172S, T172M, T172D, T172G, T172C, T172A, T172W, T172T, T172I, T172V, T172P, T172R, T172H, and T172K.

91. The method of claim 79, wherein the mutated Schizosaccharomyces pombe poly(U) polymerase substantially identical to SEQ ID NO:3 and comprises an H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W; an N171 mutation selected from the group consisting of N171E, N171L, N171Q, N171S, N171M, N171D, N171G, N171C, N171A, N171W, N171T, N171I, N171V, N171P, N171R, N171H, and N171K; and a T172 mutation selected from the group consisting T172E, T172L, T172Q, T172S, T172M, T172D, T172G, T172C, T172A, T172W, T172T, T172I, T172V, T172P, T172R, T172H, and T172K.

92. An RNA oligonucleotide prepared by the method according to any one of claims 1-52.

93. A compound of the following formula: 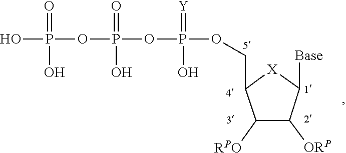
or a salt thereof, wherein:
Y is O or S;
X is O or S;
each instance of R^{P} is hydrogen, an oxygen protecting group, optionally substituted acyl, or an amino acid, or two R^{P} are joined together with the intervening atoms to form optionally substituted heterocyclyl; provided that at least one R^{P} is an oxygen protecting group, optionally substituted acyl, or an amino acid; and
“Base” is a natural or non-natural nucleotide base.

94. A compound of the following formula: 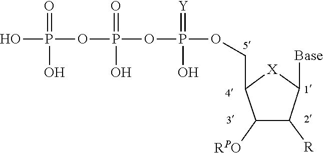
or a salt thereof, wherein:
Y is O or S;
X is O or S;
R^{P} is an oxygen protecting group, optionally substituted acyl, or an amino acid;
R is hydrogen, halogen, —CN, —NO₂, —N₃, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted hydroxyl, optionally substituted amino, or optionally substituted thiol; and
“Base” is a natural or non-natural nucleotide base.

95. A compound of the following formula: 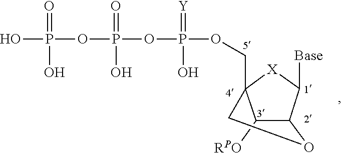
or a salt thereof, wherein:
Y is O or S;
X is O or S;
R^{P} is an oxygen protecting group, optionally substituted acyl, or an amino acid; and
“Base” is a natural or non-natural nucleotide base.

96. The compound of any one of claims 93-95, wherein R^{P} is an oxygen protecting group.

97. The compound of any one of claims 93-95, wherein R^{P} is an amino acid.

98. The compound of any one of claims 93-96, wherein R^{P} is allyl, azidomethyl, allyl carbonate, or azidomethyl carbonate.

99. The compound of any one of claims 93-96, wherein the compound is a 3′-O-allyl-NTP, 3′-O-azidomethyl-NTP, 3′-O-allyl carbonate-NTP, 3′-O-allyl carbonate-dNTP, 3′-O-azidomethyl carbonate-NTP, or 3′-O-azidomethyl carbonate-dNTP.

100. The compound of claim 93, wherein the compound is 2′-O-allyl-NTP or 2′-O-azido-methyl-NTP.

101. The compound of any one of claims 93-100, wherein the compound is of any one of the formulae recited in FIGS. 28-34.

102. A polymerase, wherein the polymerase is a mutated Schizosaccharomyces pombe poly(U) polymerase comprising mutations at one or more positions selected from H336, N171, and T172.

103. The polymerase of claim 102, wherein the mutated Schizosaccharomyces pombe poly(U) polymerase comprises an H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W.

104. The polymerase of claim 103, wherein the mutated Schizosaccharomyces pombe poly(U) polymerase comprises an H336R mutation.

105. The polymerase of claim 104, wherein the mutated Schizosaccharomyces pombe poly(U) polymerase is identical to SEQ ID NO:3, but includes an H336R mutation.

106. The polymerase of any one of claims 102-105, wherein the mutated Schizosaccharomyces pombe poly(U) polymerase comprises an N171 mutation selected from the group consisting of N171E, N171L, N171Q, N171S, N171M, N171D, N171G, N171C, N171A, N171W, N171T, N171I, N171V, N171P, N171R, N171H, and N171K.

107. The polymerase of claim 106, wherein the mutated Schizosaccharomyces pombe poly(U) polymerase comprises an N171A mutation.

108. The polymerase of claim 107, wherein the mutated Schizosaccharomyces pombe poly(U) polymerase is identical to SEQ ID NO:3, but includes an N171A mutation.

109. The polymerase of any one of claims 102-108, wherein the mutated Schizosaccharomyces pombe poly(U) polymerase comprises H336 and N171 mutations.

110. The polymerase of claim 109, wherein the mutated Schizosaccharomyces pombe poly(U) polymerase comprises H336R and N171A mutations.

111. The polymerase of claim 110, wherein the mutated Schizosaccharomyces pombe poly(U) polymerase is identical to SEQ ID NO:3, but includes H336R and N171A mutations.

112. The polymerase of claim 109, wherein the mutated Schizosaccharomyces pombe poly(U) polymerase comprises H336R and N171T mutations.

113. The polymerase of claim 112, wherein the mutated Schizosaccharomyces pombe poly(U) polymerase is identical to SEQ ID NO:3, but includes H336R and a N171T mutations.

114. The polymerase of any one of claims 102-113, wherein the mutated Schizosaccharomyces pombe poly(U) polymerase comprises a T172 mutation selected from the group consisting of T172E, T172L, T172Q, T172S, T172M, T172D, T172G, T172C, T172A, T172W, T172T, T172I, T172V, T172P, T172R, T172H, and T172K.

115. The polymerase of claim 114, wherein the mutated Schizosaccharomyces pombe poly(U) polymerase is identical to SEQ ID NO:3, but includes a T172 mutation selected from the group consisting of T172E, T172L, T172Q, T172S, T172M, T172D, T172G, T172C, T172A, T172W, T172T, T172I, T172V, T172P, T172R, T172H, and T172K.

116. The polymerase of any one of claims 102-115, wherein the mutated Schizosaccharomyces pombe poly(U) polymerase comprises H336 and T172 mutations.

117. The polymerase of claim 116, wherein the mutated Schizosaccharomyces pombe poly(U) polymerase comprises an H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W; and a T172 mutation selected from the group consisting of T172E, T172L, T172Q, T172S, T172M, T172D, T172G, T172C, T172A, T172W, T172T, T172I, T172V, T172P, T172R, T172H, and T172K.

118. The polymerase of claim 117, wherein the mutated Schizosaccharomyces pombe poly(U) polymerase is identical to SEQ ID NO:3, but includes an H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W; and a T172 mutation selected from the group consisting of T172E, T172L, T172Q, T172S, T172M, T172D, T172G, T172C, T172A, T172W, T172T, T172I, T172V, T172P, T172R, T172H, and T172K.

119. The polymerase of any one of claims 102-118, wherein the mutated Schizosaccharomyces pombe poly(U) polymerase comprises H336, N171, and T172 mutations.

120. The polymerase of claim 119, wherein the mutated Schizosaccharomyces pombe poly(U) polymerase comprises an H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W; an N171 mutation selected from the group consisting of N171E, N171L, N171Q, N171S, N171M, N171D, N171G, N171C, N171A, N171W, N171T, N171I, N171V, N171P, N171R, N171H, and N171K; and a T172 mutation selected from the group consisting T172E, T172L, T172Q, T172S, T172M, T172D, T172G, T172C, T172A, T172W, T172T, T172I, T172V, T172P, T172R, T172H, and T172K.

121. The polymerase of claim 120, wherein the mutated Schizosaccharomyces pombe poly(U) polymerase is identical to SEQ ID NO:3, but includes an H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W; an N171 mutation selected from the group consisting of N171E, N171L, N171Q, N171S, N171M, N171D, N171G, N171C, N171A, N171W, N171T, N171I, N171V, N171P, N171R, N171H, and N171K; and a T172 mutation selected from the group consisting T172E, T172L, T172Q, T172S, T172M, T172D, T172G, T172C, T172A, T172W, T172T, T172I, T172V, T172P, T172R, T172H, and T172K.

122. A kit comprising a compound of any one of claims 93-101 and/or and a polymerase of any one of claims 102-122.


SEQUENCE LISTING (8 sequences)

SEQ ID NO: 1 - protein, 494 aa, Homo sapiens
MLPKRRRARVGSPSGDAASSTPPSTRFPGVAIYLVEPRMGRSRRAFLTGLARSKGFRVLDACSSEATHVVMEETSAEEAVSWQERRMAAAPPGCTPPALLDISWLTESLGAGQPVPVECRHRLEVAGPRKGPLSPAWMPAYACQRPTPLTHHNTGLSEALEILAEAAGFEGSEGRLLTFCRAASVLKALPSPVTTLSQLQGLPHFGEHSSRVVQELLEHGVCEEVERVRRSERYQTMKLFTQIFGVGVKTADRWYREGLRTLDDLREQPQKLTQQQKAGLQHHQDLSTPVLRSDVDALQQVVEEAVGQALPGATVTLTGGFRRGKLQGHDVDFLITHPKEGQEAGLLPRVMCRLQDQGLILYHQHQHSCCESPTRLAQQSHMDAFEKSFCIFRLPQPPGAAVGGSTRPCPSWKAVRVDLVVAPVSQFPFALLGWTGSKLFQRELRRFSRKEKGLWLNSHGLFDPEQKTFFQAASEEDIFRHLGLEYLPPEQRNA

SEQ ID NO: 2 - protein, 568 aa, Saccharomyces cerevisiae
MSSQKVFGITGPVSTVGATAAENKLNDSLIQELKKEGSFETEQETANRVQVLKILQELAQRFVYEVSKKKNMSDGMARDAGGKIFTYGSYRLGVHGPGSDIDTLVVVPKHVTREDFFTVFDSLLRERKELDEIAPVPDAFVPIIKIKFSGISIDLICARLDQPQVPLSLTLSDKNLLRNLDEKDLRALNGTRVTDEILELVPKPNVFRIALRAIKLWAQRRAVYANIFGFPGGVAWAMLVARICQLYPNACSAVILNRFFIILSEWNWPQPVILKPIEDGPLQVRVWNPKIYAQDRSHRMPVITPAYPSMCATHNITESTKKVILQEFVRGVQITNDIFSNKKSWANLFEKNDFFFRYKFYLEITAYTRGSDEQHLKWSGLVESKVRLLVMKLEVLAGIKIAHPFTKPFESSYCCPTEDDYEMIQDKYGSHKTETALNALKLVTDENKEEESIKDAPKAYLSTMYIGLDFNIENKKEKVDIHIPCTEFVNLCRSFNEDYGDHKVFNLALRFVKGYDLPDEVFDENEKRPSKKSKRKNLDARHETVKRSKSDAASGDNINGTTAAVDVN

SEQ ID NO: 3 - protein, 405 aa, Schizosaccharomyces pombe
MNISSAQFIPGVHTVEEIEAEIHKNLHISKSCSYQKVPNSHKEFTKFCYEVYNEIKISDKEFKEKRAALDTLRLCLKRISPDAELVAFGSLESGLALKNSDMDLCVLMDSRVQSDTIALQFYEELIAEGFEGKFLQRARIPIIKLTSDTKNGFGASFQCDIGFNNRLAIHNTLLLSSYTKLDARLKPMVLLVKHWAKRKQINSPYFGTLSSYGYVLMVLYYLIHVIKPPVFPNLLLSPLKQEKIVDGFDVGFDDKLEDIPPSQNYSSLGSLLHGFFRFYAYKFEPREKVVTFRRPDGYLTKQEKGWTSATEHTGSADQIIKDRYILAIEDPFEISHNVGRTVSSSGLYRIRGEFMAASRLLNSRSYPIPYDSLFEEAPIPPRRQKKTDEQSNKKLLNETDGDNSE

SEQ ID NO: 4 - protein, 20 aa, Artificial Sequence
  Synthetic Polypeptide
MGSSHHHHHHSSGLVPRGSH

SEQ ID NO: 5 - RNA, 32 nt, Artificial Sequence
  Synthetic Polynucleotide
uuuuuuuuuuuuuuuuccgguaaccgguuuuu

SEQ ID NO: 6 - RNA, 32 nt, Artificial Sequence
  Synthetic Polynucleotide
uuuuuuuuuuuccgguaaccgguuuuuuuuuu

SEQ ID NO: 7 - RNA, 32 nt, Artificial Sequence
  Synthetic Polynucleotide
uccgguaaccgguuuuuuuuuuuuuuuuuuuu

SEQ ID NO: 8 - RNA, 30 nt, Artificial Sequence
  Synthetic Polynucleotide
  modified_base (1)..(1): modified by 5AmMC12
  misc_feature (13)..(13): n is ideoxyl
ucuaccauauaunacaaaacaaaauaaauu